SEQUENCE LISTING (46 sequences)

SEQ ID NO: 1 - DNA, 15 bp, synthetic construct
  source 1..15: mol_type = other DNA
gacagaactagacac

SEQ ID NO: 2 - DNA, 14 bp, synthetic construct
  source 1..14: mol_type = other DNA
acagaactagacac

SEQ ID NO: 3 - DNA, 14 bp, synthetic construct
  source 1..14: mol_type = other DNA
gacagaactagaca

SEQ ID NO: 4 - DNA, 15 bp, synthetic construct
  source 1..15: mol_type = other DNA
tgcacagctcgacgc

SEQ ID NO: 5 - DNA, 61 bp, synthetic construct
  source 1..61: mol_type = other DNA
gttctgtcatatttcagtgaatgcgagtccgtctaagagagtagtacagcaagagtgtcta

SEQ ID NO: 6 - DNA, 61 bp, synthetic construct
  source 1..61: mol_type = other DNA
gctgtgcaatatttcagtgaatgcgagtccgtctaagagagtagtacagcaagagcgtcga

SEQ ID NO: 7 - RNA, 10 nt, synthetic construct
  source 1..10: mol_type = other RNA
  modified_base 1: mod_base = OTHER; note = 2'-O-methyl modified nucleotide
  modified_base 2: mod_base = OTHER; note = Locked nucleic acid
  modified_base 3: mod_base = OTHER; note = Locked nucleic acid
  modified_base 4: mod_base = OTHER; note = Locked thymine
  modified_base 5: mod_base = OTHER; note = Locked nucleic acid
  modified_base 6: mod_base = OTHER; note = Locked nucleic acid
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
gagtccgtct

SEQ ID NO: 8 - DNA, 10 bp, synthetic construct
  source 1..10: mol_type = other DNA
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
  modified_base 8: mod_base = OTHER; note = Locked nucleic acid
cagtgaatgc

SEQ ID NO: 9 - DNA, 10 bp, synthetic construct
  source 1..10: mol_type = other DNA
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
  modified_base 7: mod_base = OTHER; note = Locked nucleic acid
gagtccgtct

SEQ ID NO: 10 - DNA, 10 bp, synthetic construct
  source 1..10: mol_type = other DNA
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
gagtccgtct

SEQ ID NO: 11 - skipped

SEQ ID NO: 12 - DNA, 11 bp, synthetic construct
  source 1..11: mol_type = other DNA
  modified_base 1: mod_base = OTHER; note = 2'-O-methyl modified nucleotide
  modified_base 2: mod_base = OTHER; note = Locked nucleic acid
  modified_base 3: mod_base = OTHER; note = Locked nucleic acid
  modified_base 4: mod_base = OTHER; note = Locked thymine
  modified_base 5: mod_base = OTHER; note = Locked nucleic acid
  modified_base 6: mod_base = OTHER; note = Locked nucleic acid
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = Amino-modified C6 nucleotide attached to 18-atom hexa-ethyleneglycol spacer attached to an amino-modified C6 dT linker attached to 3' amino modifier
  misc_feature 1..10: note = RNA
  misc_feature 11: note = DNA
gagtccgtctt

SEQ ID NO: 13 - DNA, 11 bp, synthetic construct
  source 1..11: mol_type = other DNA
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
  modified_base 8: mod_base = OTHER; note = Locked nucleic acid
  modified_base 11: mod_base = OTHER; note = Amino-modified C6 nucleotide attached to 18-atom hexa-ethyleneglycol spacer attached to an amino-modified C6 dT linker attached to 3' amino modifier
cagtgaatgct

SEQ ID NO: 14 - DNA, 11 bp, synthetic construct
  source 1..11: mol_type = other DNA
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
  modified_base 7: mod_base = OTHER; note = Locked nucleic acid
  modified_base 11: mod_base = OTHER; note = Amino-modified C6 nucleotide attached to 18-atom hexa-ethyleneglycol spacer attached to an amino-modified C6 dT linker attached to 3' amino modifier
gagtccgtctt

SEQ ID NO: 15 - DNA, 11 bp, synthetic construct
  source 1..11: mol_type = other DNA
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
  modified_base 11: mod_base = OTHER; note = Amino-modified C6 nucleotide attached to 18-atom hexa-ethyleneglycol spacer attached to an amino-modified C6 dT linker attached to 3' amino modifier
gagtccgtctt

SEQ ID NO: 16 - DNA, 61 bp, synthetic construct; identical to SEQ ID NO: 5
  source 1..61: mol_type = other DNA
  modified_base 1: mod_base = OTHER; note = 5'-Phosphorylated nucleotide

SEQ ID NO: 17 - skipped

SEQ ID NO: 18 - DNA, 15 bp, synthetic construct
  source 1..15: mol_type = other DNA
  modified_base 1: mod_base = OTHER; note = 5' thiol modifier C6 S-S modified nucleotide
gacagaactagacac

SEQ ID NO: 19 - DNA, 24 bp, synthetic construct
  source 1..24: mol_type = other DNA
  modified_base 24: mod_base = OTHER; note = Amino-modified C6 nucleotide attached to 18-atom hexa-ethyleneglycol spacer attached to an amino-modified C6 dT linker attached to 3' amino modifier
cagtgaatgcgagtccgtctaagt

SEQ ID NO: 20 - DNA, 12 bp, synthetic construct
  source 1..12: mol_type = other DNA
  modified_base 12: mod_base = OTHER; note = 3' thiol modifier C3 S-S modified nucleotide
gtagtacagcaa

SEQ ID NO: 21 - DNA, 25 bp, synthetic construct
  source 1..25: mol_type = other DNA
  modified_base 25: mod_base = OTHER; note = 3' thiol modifier C3 S-S modified nucleotide
aagagagtagtacagcagccgtcaa

SEQ ID NO: 22-28 - skipped (7 entries)

SEQ ID NO: 29 - DNA, 10 bp, synthetic construct
  source 1..10: mol_type = other DNA
  modified_base 10: mod_base = OTHER; note = Nucleotide attached to 3' Biotin
gtagtacagc

SEQ ID NO: 30 - DNA, 12 bp, synthetic construct
  source 1..12: mol_type = other DNA
  modified_base 12: mod_base = OTHER; note = Nucleotide attached to 3' Biotin
gtagtacagcaa

SEQ ID NO: 31 - DNA, 25 bp, synthetic construct
  source 1..25: mol_type = other DNA
  modified_base 25: mod_base = OTHER; note = Nucleotide attached to 3' Biotin
aagagagtagtacagcagccgtcaa

SEQ ID NO: 32 - RNA, 12 nt, synthetic construct
  source 1..12: mol_type = other RNA
  modified_base 1: mod_base = OTHER; note = 2'-O-methyl modified nucleotide
  modified_base 2: mod_base = OTHER; note = 2'-O-methyl modified thymine
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified thymine
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide attached to 3' Biotin
gtagtacagcaa

SEQ ID NO: 33-37 - skipped (5 entries)

SEQ ID NO: 38 - DNA, 15 bp, synthetic construct
  source 1..15: mol_type = other DNA
ccctccctccctccc

SEQ ID NO: 39 - DNA, 30 bp, synthetic construct
  source 1..30: mol_type = other DNA
tcgatttccttagttgtcttccttagtgag

SEQ ID NO: 40 - DNA, 40 bp, synthetic construct
  source 1..40: mol_type = other DNA
gctatgggtggtctggttgggattggccccgggagctggc

SEQ ID NO: 41 - DNA, 29 bp, synthetic construct
  source 1..29: mol_type = other DNA
ccggccaagggtgggagggagggggccgg

SEQ ID NO: 42 - DNA, 51 bp, synthetic construct
  source 1..51: mol_type = other DNA
agcgagggcggtgtccaacagcggttttttcascgaggaggttggcggtgg

SEQ ID NO: 43 - DNA, 20 bp, synthetic construct
  source 1..20: mol_type = other DNA
attgggagggattgggtggg

SEQ ID NO: 44 - protein, 6 aa, synthetic construct
HHHHHH

SEQ ID NO: 45 - DNA, 33 bp, synthetic construct
  source 1..33: mol_type = other DNA
  misc_difference 4..10: note = a, c, t, g, unknown or other
  misc_difference 4..10: note = This region may encompass 1-7 nucleotides
  misc_difference 14..20: note = a, c, t, g, unknown or other
  misc_difference 14..20: note = This region may encompass 1-7 nucleotides
  misc_difference 24..30: note = a, c, t, g, unknown or other
  misc_difference 24..30: note = This region may encompass 1-7 nucleotides
gggnnnnnnngggnnnnnnngggnnnnnnnggg

SEQ ID NO: 46 - DNA, 33 bp, synthetic construct
  source 1..33: mol_type = other DNA
  misc_difference 4..10: note = a, c, t, g, unknown or other
  misc_difference 4..10: note = This region may encompass 1-7 nucleotides
  misc_difference 14..20: note = a, c, t, g, unknown or other
  misc_difference 14..20: note = This region may encompass 1-7 nucleotides
  misc_difference 24..30: note = a, c, t, g, unknown or other
  misc_difference 24..30: note = This region may encompass 1-7 nucleotides
cccnnnnnnncccnnnnnnncccnnnnnnnccc

(19) United States
(12) Patent Application Publication
(10) Pub. No.: US 2024/0229110 A1
(43) Pub. Date: Jul. 11, 2024

(54) ASSAY METHODS AND KITS

(71) Applicant: MESO SCALE TECHNOLOGIES, LLC., Rockville, MD (US)

(72) Inventors: John KENTEN, Boyds, MD (US); Galina NIKOLENKO, Damascus, MD (US); John Robert PETTERSSON, Rockville, MD (US)

(21) Appl. No.: 18/544,734

(22) Filed: Dec. 19, 2023

Related U.S. Application Data
(60) Provisional application No. 63/497,224, filed on Apr. 20, 2023.
(60) Provisional application No. 63/476,179, filed on Dec. 20, 2022.

(51) Int. Cl. C12Q 1/6825; C12Q 1/682

(52) U.S. Cl. CPC C12Q 1/6825; C12Q 1/682; C12Q 1/6806

(57) ABSTRACT

The invention provides novel components for conducting assays, e.g., sandwich immunoassays, methods utilizing the novel components, and compositions and kits comprising the novel components.

### SEQUENCE LISTING

The instant application contains a Sequence Listing which has been submitted electronically in XML file format and is hereby incorporated by reference in its entirety. Said XML copy, created on Dec. 18, 2023, is named 0076-0065US1_SL.xml and is 80,962 bytes in size.

### FIELD OF THE INVENTION

The invention provides components for conducting assays, e.g., sandwich immunoassays, methods utilizing the components, and compositions and kits comprising the components.

### BACKGROUND

Immunoassays, e.g., sandwich immunoassays, are commonly used to detect analytes in a sample. Methods to improve assay sensitivity often involve analyte-dependent multi-step optimization procedures that require numerous additional assay components and/or instrumentation, which increase complexity and cost. Moreover, the optimized assays can require longer run times and/or complicated analysis methods.

### SUMMARY OF THE INVENTION

In embodiments, the invention provides a method for detecting an analyte, comprising: (a) contacting a template oligonucleotide with a first complex that comprises: (1) the analyte; and (2) a detection reagent that binds the analyte, wherein the detection reagent comprises a nucleic acid primer; (b) hybridizing the nucleic acid primer to the template oligonucleotide to form a second complex and extending the nucleic acid primer with a polymerase, thereby forming an extended oligonucleotide; (c) binding the extended oligonucleotide to one or more labeled probes, wherein each labeled probe comprises (1) a detection oligonucleotide that is capable of binding to the extended oligonucleotide; and (2) a detectable label; and (d) detecting the detectable label, thereby detecting the analyte, wherein the second complex is bound to a surface, and wherein: (i) the detection oligonucleotide comprises RNA, a modified nucleic acid, or a combination thereof; (ii) the method further comprises terminating the extending by cleaving the template oligonucleotide; (iii) combination of (i) and (ii); (iv) one or both of (i) and (ii), further wherein the surface comprises an anchoring reagent; (v) the surface comprises an anchoring reagent that comprises an anchoring oligonucleotide, wherein the anchoring oligonucleotide comprises a modified nucleic acid; or (vi) any combination of (i), (ii), and (v).

In embodiments, the invention provides a kit for detecting an analyte, comprising, in one or more vials, containers, or compartments: (a) a capture reagent that binds the analyte; (b) a detection reagent that binds the analyte, wherein the detection reagent comprises a nucleic acid primer or is capable of being linked to a nucleic acid primer; (c) a labeled probe that comprises (1) a detection oligonucleotide and (2) a detectable label; and (d) a template oligonucleotide that is capable of hybridizing to the nucleic acid primer and that comprises a same sequence as the detection oligonucleotide; wherein the detection reagent comprises a protein or polypeptide, and wherein: (i) the detection oligonucleotide comprises RNA, a modified nucleic acid, or a combination thereof; (ii) the kit further comprises a nuclease that is capable of cleaving the template oligonucleotide; (iii) combination of (i) and (ii); (iv) one or both of (i) and (ii), and wherein the kit further comprises an anchoring reagent; (v) the kit further comprises an anchoring reagent that comprises an anchoring oligonucleotide, wherein the anchoring oligonucleotide comprises a modified nucleic acid; or (vi) any combination of (i), (ii), and (v).

In embodiments, the invention provides a composition for labeling a surface, comprising: a labeled probe that comprises (1) a detectable label; and (2) detection oligonucleotide that is capable of binding to an extended oligonucleotide that is bound to the surface, wherein the extended oligonucleotide is formed by extension of a nucleic acid primer by a polymerase based on a template oligonucleotide, and wherein: (i) the detection oligonucleotide comprises RNA, a modified nucleic acid, or a combination thereof; (ii) the composition further comprises a nuclease that is capable of cleaving the template oligonucleotide; (iii) combination of (i) and (ii); (iv) one or both of (i) and (ii), and further wherein the extended oligonucleotide is bound to the surface via an anchoring reagent; (v) the surface comprises an anchoring reagent that comprises an anchoring oligonucleotide, wherein the anchoring oligonucleotide comprises a modified nucleic acid; or (vi) any combination of (i), (ii), and (v). In embodiments, the template oligonucleotide is a circular oligonucleotide. In embodiments, the extension of the nucleic acid primer is by rolling circle amplification (RCA).

In embodiments, the invention provides a composition for labeling a surface, comprising: (a) a nucleic acid primer that is immobilized directly or indirectly on a surface; (b) a template oligonucleotide comprising (1) a first region that is complementary to the nucleic acid primer; and (2) a second region that comprises a same sequence as a detection oligonucleotide; (c) a polymerase; and (d) a labeled probe comprising (1) a detectable label; and (2) detection oligonucleotide that is capable of binding to an extended oligonucleotide that is bound to the surface, wherein an extended oligonucleotide is formed by extension of the nucleic acid primer by the polymerase based on the template oligonucleotide, and wherein: (i) the detection oligonucleotide comprises RNA, a modified nucleic acid, or a combination thereof; (ii) the composition further comprises a nuclease that is capable of cleaving the template oligonucleotide; (iii) combination of (i) and (ii); (iv) one or both of (i) and (ii), and further wherein the extended oligonucleotide is bound to the surface via an anchoring reagent; (v) the surface comprises an anchoring reagent that comprises an anchoring oligonucleotide, wherein the anchoring oligonucleotide comprises a modified nucleic acid; or (vi) any combination of (i), (ii), and (v). In embodiments, the template oligonucleotide is a circular oligonucleotide. In embodiments, the extension of the nucleic acid primer is by rolling circle amplification (RCA).

In embodiments, the invention provides a composition comprising: a capture reagent, an analyte, a detection reagent that comprises a nucleic acid primer, a template oligonucleotide, a polymerase, and a nuclease, wherein: the capture reagent is immobilized on the surface; the capture reagent and the detection reagent are bound to the analyte; the nucleic acid primer is hybridized to the template oligonucleotide; the polymerase is capable of extending the nucleic acid primer; and the nuclease is capable of cleaving the template oligonucleotide.

In embodiments, the invention provides a composition comprising: a capture reagent, an analyte, a detection reagent that comprises an extended oligonucleotide, and an anchoring reagent that comprises an anchoring oligonucleotide, wherein: the capture reagent and the anchoring reagent are immobilized on the surface; the capture reagent and the detection reagent are bound to the analyte; the anchoring oligonucleotide comprises a modified nucleic acid selected from a peptide nucleic acid (PNA), a locked nucleic acid (LNA), a bridged nucleic acid (BNA), a nucleoside comprising a 2′ modification, or a combination thereof, optionally wherein the nucleoside comprising the 2′ modification comprises a 2′-O-methyl modification (2′-OMe), a 2′-O-methoxyethyl modification (2′-MOE), a 2′-deoxy-2′-fluoro modification (2′-F), a 2′-hydroxyl modification (2′-OH), or a combination thereof; and the extended oligonucleotide comprises an anchoring complement that is bound to the anchoring oligonucleotide.

In embodiments, the invention provides a composition comprising: a capture reagent, an analyte, a detection reagent that comprises an extended oligonucleotide, and a labeled probe that comprises a detection oligonucleotide, wherein: the capture reagent and the detection reagent are bound to the analyte; the detection oligonucleotide comprises RNA, a modified nucleic acid, or a combination thereof, wherein the modified nucleic acid is selected from a PNA, an LNA, a BNA, a nucleoside comprising a 2′ modification, or a combination thereof, optionally wherein the nucleoside comprising the 2′ modification comprises a 2′-O-methyl modification (2′-Ome), a 2′-O-methoxyethyl modification (2′-MOE), a 2′-deoxy-2′-fluoro modification (2′-F), a 2′-hydroxyl modification (2′-OH), or a combination thereof; and the extended oligonucleotide is bound to the detection oligonucleotide.

In embodiments, the invention provides an oligonucleotide comprising any one of SEQ ID NOs:7-15 or SEQ ID NO:17. In embodiments, the invention provides an oligonucleotide consisting of any one of SEQ ID NOs:7-15 or SEQ ID NO:17.

### BRIEF DESCRIPTION OF THE DRAWINGS

The following drawings form part of the specification and are included to further demonstrate exemplary embodiments of certain aspects of the invention.

FIGS. 1A and 1B show exemplary modified nucleic acids according to embodiments herein. FIG. 1A is adapted from Duffy et al., BMC Biology 18:112 (2020). FIG. 1B shows exemplary bridged nucleic acids (BNAs).

FIG. 2A shows representative results of a comparative assay performed with a detection oligonucleotide of 23 nucleotides in length and without any modified nucleic acids (“DNA-23”) and a detection oligonucleotide of 10 nucleotides in length and includes 6 locked nucleic acids (LNAs) (“LNA-10/6”), as described in embodiments herein. FIG. 2B shows representative results of a comparative assay performed with a detection oligonucleotide of 23 nucleotides in length and without any modified nucleic acids (“Detect23”) and a detection oligonucleotide of 10 nucleotides in length and includes 5 LNAs (“Detect10+5L(A)”), as described in embodiments herein.

FIGS. 3A-3D show representative results of an assay performed according to embodiments herein, where restriction enzymes DdeI, HpaII, AluI, and StuI, shown respectively in FIGS. 3A-3D, were added to cleave the template oligonucleotide as described herein.

FIG. 4 shows representative results of a calibration assay performed with varying concentrations of DdeI for cleavage of the template oligonucleotide, as described in embodiments herein. The Hill slopes of the assays are also shown.

FIG. 5 shows representative results of a kinetics assay performed with three different concentrations of DdeI (0, 0.005, or 0.05 U/well) and at three different temperatures (20° C., 23.5° C., and 27° C.), as described in embodiments herein.

FIGS. 6A-6B show representative results of an assay where the addition of the restriction enzyme ApoI significantly reduces the temperature dependence of the assay. FIG. 6A shows ECL signal results. FIG. 6B shows ECL signal following normalization of the signal to the ECL signal at 1 hour, 27° C.

FIGS. 7A-7C show representative results of two assays performed at room-temperature (23.5° C.), where the addition of the restriction enzyme TspRI improves the ECL signal stability over time. FIG. 7A shows the ECL signal for detection of a biotinylated primer on streptavidin.

FIG. 7B shows the ECL signal following normalization of the signal to the ECL signal at the 1 hour time point. FIG. 7C shows the ECL signal for an IL-5 immunoassay normalized to ECL signal at the 1 hour time point.

FIGS. 8A-8B show the dependence of the anchoring oligonucleotide (“Anchor”) length on the background signals generated from samples containing anti-single stranded DNA antibodies and the improvements on the addition of ssDNA to block this sample interferences. The Anchors included the following modified nucleotides: 2′-O-methylated (A12-OM) and locked nucleic acid bases LNA9-1, LNA9-2, LNA9-3, LNA 9-6, LNA 9-7, LNA 9-8, LNA 9-9 and A9+3L.

FIGS. 9A-9B show the stabilization of the ECL signal, expressed as the percentage of ECL signal retained as washer speed is increased. In FIG. 9A, the shorter A9+3L6OM Anchors, containing LNA and 2′-OMe bases, demonstrate similar stability to the A25 DNA based Anchors shown in FIG. 9B.

FIGS. 10A-10B show both reduced non-specific signal and range (Max to Min) of non-specific signals from the use of Anchors with modified bases (A9+3L6OM (and PEG spacers) and A9+4L) vs the DNA based Anchors (A25), when used in combination with ssDNA in the sample diluent. FIG. 10B shows the results with A9-NoPeg, A9-1Peg and A9-2Peg, where A9 is the same as the A9+3L6OM from FIG. 9A.

FIG. 11A shows the relative stability of three anchoring oligonucleotides: (i) DNA based (A25), (ii) LNA based (A9+4L), and (iii) LNA, 2′-OMe based (A9+3L6OM), at differing washer speeds, illustrating the improved performance of the modified Anchors in a shorter sequence.

FIG. 11B shows representative results of an assay performed according to embodiments herein, where an anchoring oligonucleotide of 25 nucleotides in length and without any modified nucleic acids (“A25”) with varying concentrations of a primer and either a detection oligonucleotide of 23 nucleotides in length and without any modified nucleic acids (“D23”), or a detection oligonucleotide of 10 nucleotides in length and including LNAs and/or 2′-OMe modified nucleic acids as described herein (“D10A+D10B”).

FIG. 11C shows representative results of an assay performed according to embodiments herein, where an anchoring oligonucleotide of 9 nucleotides in length and including LNAs and 2′-OMe nucleotides (“A9+3L6OM”) with varying concentrations of a primer and either a detection oligonucleotide of 23 nucleotides in length and without any modified nucleic acids (“D23”), or a detection oligonucleotide of 10 nucleotides in length and including LNAs and/or 2′-OMe modified nucleic acids as described herein (“D10A+D10B”).

FIG. 11D shows representative results of an assay performed according to embodiments herein, where the primer was added at two concentrations, and either 0.05 U/well or 0.1 U/well DdeI was added for cleavage of the template oligonucleotide, and a detection oligonucleotide of 10 nucleotides in length and including LNAs and/or 2′-OMe modified nucleic acids (“D10A+D10B”).

FIG. 11E shows representative results of an assay performed according to embodiments herein, where TspRI was added for cleavage of the template oligonucleotide, an anchoring oligonucleotide (A9+3L6OM including LNAs and/or 2′-OMe modified nucleic acids) and a detection oligonucleotide of 10 nucleotides in length and including LNAs and/or 2′-OMe modified nucleic acids (“D10A+D10B”) was added. This illustrated the improved stability of the ECL signal to washer flow rates from this combination of assay improvements.

FIG. 12 shows representative results of assays performed according to embodiments herein, with different detection oligonucleotide and template oligonucleotide combinations. D10A+5L: 10-nucleotide (nt) detection oligonucleotide with 5 LNAs; used with a 61-nt template. D10A+5L5OM: 10-nt detection oligonucleotide with 5 LNAs and 5 2′-OMe nucleotides; used with a 61-nt template. D10A+5L-58A: 10-nt detection oligonucleotide with 5 LNAs; used with a 58-nt template. D10A+5L5OM-58A: 10-nt detection oligonucleotide with 5 LNAs and 5 2′-OMe nucleotides; used with a 58-nt template. D10A+5L/D10B+6L: Mixture of D10A+5L and D10B+6L; used with a 61-nt template. D10A+5L5OM/D10B+6L: Mixture of D10A+5L5OM and D10B+6L; used with a 61-nt template. NSB: non-specific binding.

FIG. 13 shows representative results of assays performed with addition of a single stranded oligonucleotide (SSO) stabilizing agent, Extreme Thermostable Single-Stranded DNA Binding Protein (ET SSB) during the extension reaction. The top panel shows the results of adding ET SSB before the polymerase. The bottom panel shows the results of adding ET SSB after the polymerase.

FIG. 14 shows representative results of assays performed with different concentrations of ET SSB. The 4 panels show addition of ET SSB 0, 5, 15, or 60 minutes after the polymerase.

FIG. 15 shows representative results of the fold assay signal increase over 24 hours of extension when ET SSB is added 0, 5, 15, or 60 minutes after the polymerase.

FIG. 16 shows representative results of the ratio of the assay signal at 24 hours to 4 hours in the presence of ET SSB or a control reaction without ET SSB.

FIG. 17 shows an exemplary illustration of an embodiment herein. In the left panel of FIG. 17, an anchoring reagent portion of the capture reagent-anchoring reagent hybrid comprises a first binding partner, which binds to a second binding partner on the surface. In the right panel of FIG. 17, a capture reagent portion of the capture reagent-anchoring reagent hybrid is immobilized directly onto the surface.

FIG. 18 shows an exemplary illustration of an embodiment herein. A capture reagent and an anchoring reagent (“anchor”) are immobilized on a surface. The capture reagent is complexed with an analyte, a first detection reagent comprising a first nucleic acid probe (“PC1”), and a second detection reagent comprising a second nucleic acid probe (“PC2”). A bridging oligonucleotide (“BO”), which comprises a nucleic acid primer, hybridizes to both PC1 and PC2. The nucleic acid primer of the BO is capable of hybridizing to the template oligonucleotide and extended to form an extended oligonucleotide. A labeled probe, which is capable of binding to the extended oligonucleotide, is also shown.

FIGS. 19A and 19B show exemplary illustrations of embodiments herein. In FIGS. 19A and 19B, a first complex is formed comprising a capture reagent, an analyte (“A”), and a detection reagent, and an extended oligonucleotide is formed from a nucleic acid primer on the detection reagent. The extended oligonucleotide comprises a binding sequence. In FIG. 19A, the binding sequence is capable of binding to the surface, which may comprise an anchoring reagent. In FIG. 19B, the anchoring reagent is linked to a further capture reagent on the surface, and the binding sequence binds to the anchoring reagent linked to the capture reagent.

FIG. 20 shows an exemplary illustration of an embodiment herein. A first complex is formed comprising a capture reagent, an analyte (“A”), and a detection reagent, and an extended oligonucleotide is formed from a nucleic acid primer on the detection reagent. The extended oligonucleotide comprises a binding moiety (e.g., a hapten, which may be incorporated into the extended oligonucleotide via a hapten-labeled dNTP), which binds to an anchoring reagent that binds the binding moiety (e.g., a protein or antibody that binds the hapten).

FIG. 21 shows an exemplary illustration of an embodiment herein. A first detection reagent comprising a first nucleic acid primer hybridizes to a first region of the template oligonucleotide, and a second detection reagent comprising a second nucleic acid primer hybridizes to a second region of the template oligonucleotide. Both the first and second nucleic acid primers are capable of being extended, thereby forming first and second extended oligonucleotides as described herein.

FIG. 22 shows an exemplary illustration of an embodiment herein. A first complex is formed comprising a capture reagent, an analyte (“A”), and a detection reagent, and an extended oligonucleotide is formed from a nucleic acid primer on the detection reagent. The extended oligonucleotide is capable of forming a secondary structure comprising a detectable enzymatic activity (e.g., an aptamer that binds hemin). The enzymatic activity, e.g., peroxidase activity, is detected, thereby detecting the analyte as described herein.

FIG. 23 shows an exemplary illustration of an embodiment herein. Anchoring reagents are immobilized on a surface, e.g., a Western blot membrane, and a first complex is formed on the surface comprising an analyte and first and second detection reagents that comprise first and second nucleic acid primers, respectively. The first and second nucleic acid primers hybridize to a template oligonucleotide, which may be ligated to form a circular template. The first or second nucleic acid primer is extended to form an extended oligonucleotide. The extended oligonucleotide is capable of binding to a detectably labeled probe.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional unrecited elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y.

As used herein, the term “simultaneous” in reference to one or more events (e.g., contacting a template oligonucleotide with a polymerase and a nuclease) means that the events occur at exactly the same time or at substantially the same time, e.g., simultaneous events described herein can occur less than or about 10 minutes apart, less than or about 5 minutes apart, less than or about 2 minutes apart, less than or about 1 minute apart, less than or about 30 seconds apart, less than or about 15 seconds apart, or less than or about 5 seconds apart.

As used herein, the term “level” in the context of an analyte refers to the amount, concentration, or biological activity or chemical reactivity (collectively referred to as “activity”) of the analyte. The term “level” can also refer to the rate of change of the amount, concentration, or activity of a biomarker. “Level” can also refer to an absolute amount of an analyte in a sample or to a relative amount of the analyte, including amount or concentration determined under steady-state or non-steady-state conditions. “Level” can further refer to an assay signal that correlates with the amount, concentration, activity or rate of change of an analyte. The level of an analyte can be determined relative to a control component in a sample.

As used herein, a “nucleic acid” refers to one (i.e., a single nucleotide monomer) or more nucleotides. In embodiments where “nucleic acid” refers to more than one nucleotide, the nucleotides may be covalently linked to form a polymeric structure, e.g., a “polynucleotide,” “oligonucleotide,” or “nucleic acid sequence.” The term “nucleic acid” includes ribonucleic acid (RNA), e.g., an RNA nucleotide monomer, and deoxyribonucleic acid (DNA), e.g., a DNA nucleotide monomer. A “nucleotide” includes a nucleobase and sugar, which collectively form a “nucleoside,” and a phosphate. A “standard” nucleotide referred to herein is a nucleotide with an adenine, guanine, thymine, uracil, or cytosine base, a deoxyribose or ribose sugar, and a phosphate.

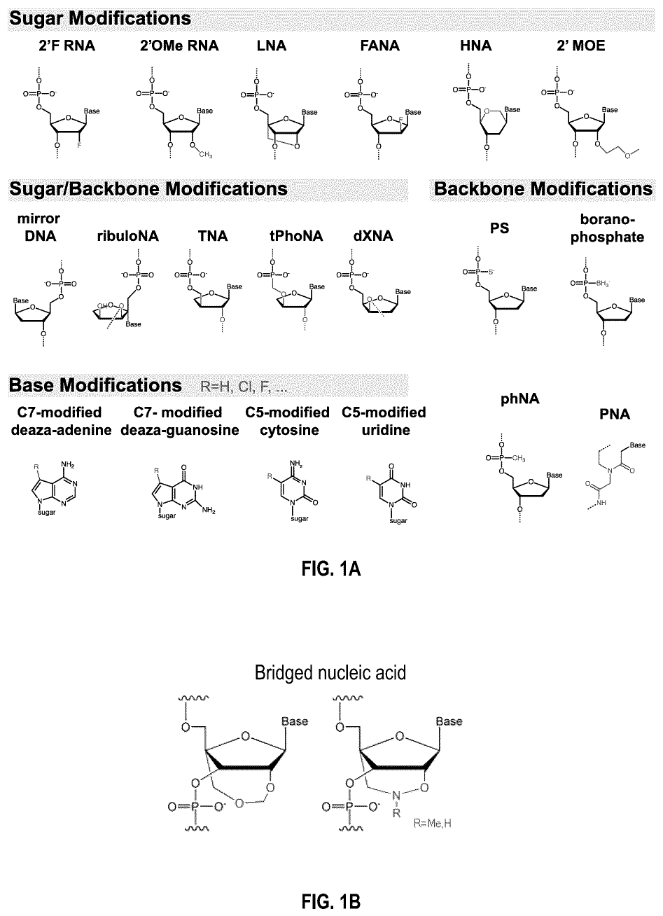

As used herein, a “modified nucleic acid” refers to one (i.e., a single nucleotide monomer) or more nucleotides that comprises a modification in the base, sugar, and/or backbone of a standard nucleotide. In embodiments, the modified nucleic acid comprises a naturally occurring modification, e.g., methylation of the 2′-O atom of the ribose ring, a modification at C5 of a pyrimidine base, and the like, which may be found in natural oligonucleotides but are not part of a standard DNA or RNA nucleotide. In embodiments, the modified nucleic acid comprises a non-naturally occurring modification, e.g., a locked nucleic acid monomer (LNA) or peptide nucleic acid monomer (PNA), which can be synthesized using techniques known in the field. Exemplary modified nucleic acids are further described in, e.g., Duffy et al., BMC Biology 18:112 (2020), and shown in FIG. 1A, adapted from Duffy et al., BMC Biology 18:112 (2020).

A “peptide nucleic acid” or “PNA” refers to DNA mimic in which the deoxyribose phosphate backbone is replaced by a pseudo-peptide polymer to which the nucleobases are linked. As used herein, the terms “peptide nucleic acid” and “PNA” encompass a single PNA monomer or more than one PNA monomers. In general, PNAs hybridize with complementary DNAs or RNAs, e.g., extended oligonucleotides described herein, with higher affinity and specificity as compared to a non-modified, naturally occurring nucleic acid (e.g., DNA or RNA). PNAs are further described in, e.g., Pellestor et al, European Journal of Human Genetics 12:694-700 (2004).

A “locked nucleic acid” or “LNA” refers to a modified RNA nucleotide in which the ribose ring is “locked” with a methylene bridge connecting the 2′-O atom with the 4′-C atom. As used herein, the terms “locked nucleic acid” and “LNA” encompass a single LNA monomer or more than one LNA monomers. Non-limiting examples of LNAs are described, e.g., in Beigelman et al., Nucleic Acids Research 23(21):4434-4442 (1995).

A “bridged nucleic acid” or “BNA” refers to a modified RNA nucleotide that contains a bridged connection between the 2′-O atom and the 4′-C atom of the ribose. As used herein, the terms “bridged nucleic acid” or “BNA” encompass a single BNA monomer or more than one BNA monomers. In embodiments, the bridged connection of a BNA comprises an amine, a sulfur, an oxygen, or combination thereof. Examples of BNAs are shown in FIG. 1B. Further exemplary BNAs include, but are not limited to, 3′-amino-2′,4′-BNA; 2′,4′-BNA-2-pyridone; 2′,4′-ENA, 2′,4′-BNA-1-isoquinolone; 2′,4′-BNA^{NC}[NH]; 2′,4′-BNA^{NC}[NMe]; and 2′,4′-BNA^{NC}[NBn]. See, e.g., Obika et al., Tetrahedron Letters 38(50):8735-8738 (1997); Koshkin et al., Tetrahedron 54(14):3607-3630 (1998); Obika et al., Chemical Communications 19:1992-1993 (2001); and Soler-Bistué et al., Molecules 24(12):2297 (2019).

In embodiments, the invention provides assays, e.g., sandwich immunoassays, that utilize novel components to improve assay sensitivity, increase assay signal, decrease non-specific background signal and non-specific binding interactions of the assay components, and/or improve robustness of the assay to variations in temperature and/or assay run time. In embodiments, the assay comprises: extending a nucleic acid primer to form an extended oligonucleotide; binding the extended oligonucleotide to an anchoring oligonucleotide; binding a labeled probe comprising a detection oligonucleotide to the extended oligonucleotide; and detecting the labeled probe bound to the extended oligonucleotide, wherein the amount of extended oligonucleotide corresponds to the amount of analyte. In embodiments, the invention provides a detection oligonucleotide, e.g., that comprises RNA, a modified nucleic acid, or combination thereof. In embodiments, the invention provides an anchoring oligonucleotide, e.g., comprising a modified nucleic acid. In embodiments, the invention provides an efficient extending step, e.g., by cleaving the template oligonucleotide. In embodiments, the invention provides compositions and kits comprising the components disclosed herein.

In embodiments, the invention provides a method for detecting an analyte, comprising:
(a) contacting a template oligonucleotide with a first complex that comprises: (1) the analyte; and (2) a detection reagent that binds the analyte, wherein the detection reagent comprises a nucleic acid primer;
(b) hybridizing the nucleic acid primer to the template oligonucleotide to form a second complex and extending the nucleic acid primer with a polymerase, thereby forming an extended oligonucleotide;
(c) binding the extended oligonucleotide to one or more labeled probes, wherein each labeled probe comprises (1) a detection oligonucleotide that is capable of binding to the extended oligonucleotide; and (2) a detectable label; and
(d) detecting the detectable label, thereby detecting the analyte, wherein the second complex is bound to a surface, and wherein:
   (i) the detection oligonucleotide comprises RNA, a modified nucleic acid, or a combination thereof;
   (ii) the method further comprises terminating the extending by cleaving the template oligonucleotide;
   (iii) combination of (i) and (ii);
   (iv) one or both of (i) and (ii), further wherein the surface comprises an anchoring reagent;
   (v) the surface comprises an anchoring reagent that comprises an anchoring oligonucleotide, wherein the anchoring oligonucleotide comprises a modified nucleic acid; or
   (vi) any combination of (i), (ii), and (v).

In embodiments, the detection oligonucleotide comprises RNA, a modified nucleic acid, or a combination thereof; and the method comprises terminating the extending by cleaving the template oligonucleotide. In embodiments, the detection oligonucleotide comprises RNA, a modified nucleic acid, or a combination thereof; and the surface comprises an anchoring reagent. In embodiments, the method comprises terminating the extending by cleaving the template oligonucleotide; and the surface comprises an anchoring reagent. In embodiments, the detection oligonucleotide comprises RNA, a modified nucleic acid, or a combination thereof; the method comprises terminating the extending by cleaving the template oligonucleotide; and the surface comprises an anchoring reagent. In embodiments, the anchoring reagent comprises an anchoring oligonucleotide, wherein the anchoring oligonucleotide comprises a modified nucleic acid. In embodiments, the detection oligonucleotide comprises a sequence of any one of SEQ ID NOs:7-10. In embodiments, the cleaving comprises contacting a nuclease with the template oligonucleotide, wherein the nuclease comprises one or more restriction enzymes as shown in Table 1. In embodiments, the nuclease comprises DdeI, AluI, HpaII, ApoI, DpnI, DpnII, ScrFI, MboI, MluCI, AseI, TaqI, or combination thereof. In embodiments, the template oligonucleotide comprises a sequence of any one of SEQ ID NOs:5, 6, or 16. In embodiments, the anchoring oligonucleotide comprises a sequence of any one of SEQ ID NOs:11, 17, and 21-37.

TABLE 1
Exemplary Restriction Enzymes

| Cleavage Site | |
|---|---|
| Length (nt) | Restriction Enzyme |
| 4 | AluI, BfaI, CviII, CviKI01, CviQI, DpnII, FatI, |
| | HpaII, HpyCH4IV, MboI, MluCI, MseI, MspI, |
| | NlaIII, RsaI, Sau3AI, TaqI-v2 |
| 5 | AvaII, BstMutI, DdeI, HinfI, Hpy188I, NciI, |
| | Sau96I, ScrFI, TspRI |
| 6 | Apo I, AseI, AvaI, BsaAI, BsmI, BsrI, PmlI, |
| | PvuI, SmaI, StuI, XmaI |

In embodiments, the detection oligonucleotide comprises a sequence of any one of SEQ ID NOs:7-10; and one or both of:
the method comprises terminating the extending by cleaving the template oligonucleotide, e.g., comprising a sequence of any one of SEQ ID NOs:5, 6, or 16; and
the surface comprises an anchoring reagent.

In embodiments, the method comprises terminating the extending by cleaving the template oligonucleotide, e.g., comprising a sequence of any one of SEQ ID NOs:5, 6, or 16; wherein the cleaving comprises contacting a nuclease with the template oligonucleotide, wherein the nuclease comprises DdeI, AluI, HpaII, ApoI, DpnI, DpnII, ScrFI, MboI, MluCI, AseI, TaqI, TspRI, or combination thereof; and one or both of:
the detection oligonucleotide comprises RNA, a modified nucleic acid, or a combination thereof; and
the surface comprises an anchoring reagent.

In embodiments, the surface comprises an anchoring reagent, wherein the anchoring reagent comprises an anchoring oligonucleotide comprising a sequence of any one of SEQ ID NOs:11, and 21-37, and one or both of:
the detection oligonucleotide comprises RNA, a modified nucleic acid, or a combination thereof; and
the method comprises terminating the extending by cleaving the template oligonucleotide, e.g., comprising a sequence of any one of SEQ ID NOs:5, 6, or 16.

In embodiments, the detection oligonucleotide comprises a sequence of any one of SEQ ID NOs:7-10;
the method comprises terminating the extending by cleaving the template oligonucleotide, e.g., comprising a sequence of any one of SEQ ID NOs:5, 6, or 16, wherein the cleaving comprises contacting a nuclease with the template oligonucleotide, wherein the nuclease comprises DdeI, AluI, HpaII, ApoI, DpnI, DpnII, ScrFI, MboI, MluCI, AseI, TaqI, TspRI, or combination thereof; and
the surface comprises an anchoring reagent.

In embodiments, the detection oligonucleotide comprises a sequence of any one of SEQ ID NOs:7-10;
the method comprises terminating the extending by cleaving the template oligonucleotide, e.g., comprising a sequence of any one of SEQ ID NOs:5, 6, or 16; and
the surface comprises an anchoring reagent, wherein the anchoring reagent comprises an anchoring oligonucleotide comprising a sequence of any one of SEQ ID NO:11, 17, and 21-37.

In embodiments, the detection oligonucleotide comprises RNA, a modified nucleic acid, or a combination thereof;
the method comprises terminating the extending by cleaving the template oligonucleotide, e.g., comprising a sequence of any one of SEQ ID NOs:5, 6, or 16, wherein the cleaving comprises contacting a nuclease with the template oligonucleotide, wherein the nuclease comprises DdeI, AluI, HpaII, ApoI, DpnI, DpnII, ScrFI, MboI, MluCI, AseI, TaqI, TspRI, or combination thereof; and
the surface comprises an anchoring reagent, wherein the anchoring reagent comprises an anchoring oligonucleotide comprising a sequence of any one of SEQ ID NO:11, 17, and 21-37.

In embodiments, the detection oligonucleotide comprises a sequence of any one of SEQ ID NOs:7-10;
the method comprises terminating the extending by cleaving the template oligonucleotide, e.g., comprising a sequence of any one of SEQ ID NOs:5, 6, or 16, wherein the cleaving comprises contacting a nuclease with the template oligonucleotide, wherein the nuclease comprises DdeI, AluI, HpaII, ApoI, DpnI, DpnII, ScrFI, MboI, MluCI, AseI, TaqI, TspRI, or combination thereof; and
the surface comprises an anchoring reagent, wherein the anchoring reagent comprises an anchoring oligonucleotide comprising a sequence of any one of SEQ ID NO:11, 17, and 21-37.

### Detection Reagent

In embodiments, the method comprises contacting a template oligonucleotide with a first complex that comprises the analyte and a detection reagent. In embodiments, the detection reagent specifically binds to the analyte. In embodiments, the detection reagent comprises a nucleic acid primer.

In embodiments, the detection reagent comprises a protein or polypeptide, antibody or antigen-binding fragment thereof, antigen, ligand, receptor, oligonucleotide, hapten, epitope, mimotope, or aptamer. In embodiments, the detection reagent comprises an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent comprises an antibody or antigen-binding fragment thereof. In embodiments, the detection reagent comprises an antigen-binding domain that specifically binds to an epitope of the analyte. In embodiments, the detection reagent comprises an oligonucleotide. In embodiments, the analyte comprises an oligonucleotide, and the detection reagent and the analyte comprise complementary oligonucleotides.

### Nucleic Acid Primer

In embodiments, the detection reagent is linked to a nucleic acid primer. In embodiments, the detection reagent comprises an oligonucleotide, and the nucleic acid primer is at a 5′ end or 3′ end of the oligonucleotide. In embodiments, the detection reagent comprises an antibody or antigen-binding fragment thereof, and the nucleic acid primer is conjugated to the detection reagent. Conjugation of nucleic acids with biomolecules such as antibodies or antigen-binding fragments thereof are known to one of ordinary skill in the art. For example, conjugation of nucleic acid primers to detection reagents is described in WO 2020/180645.

In embodiments, the nucleic acid primer is about 10 to about 30 nucleotides in length, or about 12 to about 28 nucleotides in length, or about 13 to about 26 nucleotides in length, or about 14 to about 24 nucleotides in length, or about 11 to about 22 nucleotides in length, or about 12 to about 21 nucleotides in length, or about 13 to about 20 nucleotides in length, or about 13 to about 18 nucleotides in length, or about 14 to about 19 nucleotides in length. In embodiments, the nucleic acid primer is about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides in length.

In embodiments, the nucleic acid primer is about 14 nucleotides in length or about 15 nucleotides in length. In embodiments, the nucleic acid primer comprises or consists of a sequence shown in Table 2.

TABLE 2
Exemplary nucleic acid primer sequences

| | |
|---|---|
| 5′-GACAGAACTAGACAC-3′ | SEQ ID NO: 1 |
| 5′-ACAGAACTAGACAC-3′ | SEQ ID NO: 2 |
| 5′-GACAGAACTAGACA-3′ | SEQ ID NO: 3 |
| 5′-TGCACAGCTCGACGC-3′ | SEQ ID NO: 4 |

In embodiments, the invention provides an oligonucleotide comprising a sequence of any one of SEQ ID NOs:1-4. In embodiments, the invention provides an oligonucleotide consisting of a sequence of any one of SEQ ID NOs:1-4.

### Template Oligonucleotide

In embodiments, the nucleic acid primer comprises a complementary region to the template oligonucleotide. In embodiments, the template oligonucleotide is a template for nucleic acid amplification, e.g., by polymerase chain reaction (PCR); nicking and extension amplification reaction (NEAR); an isothermal amplification method, such as strand displacement amplification (SDA), helicase-dependent amplification (HDA) or rolling circle amplification (RCA); or combinations thereof.

In embodiments, the template oligonucleotide is about 40 to about 100 nucleotides in length, or about 50 to about 78 nucleotides in length, or about 53 to about 76 nucleotides in length, or about 50 to about 70 nucleotides in length, or about 53 to about 61 nucleotides in length, or about 54 to about 61 nucleotides in length. In embodiments, the template oligonucleotide is about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, or about 76 nucleotides in length.

In embodiments, the template oligonucleotide comprises the sequence 5′-GTTCTGTC-3′ at its 5′ end and the sequence 5′-GTGTCTA-3′ at its 3′ end. In embodiments, the template oligonucleotide comprises or consists of a sequence shown in Table 3.

TABLE 3
Exemplary template oligonucleotide sequences

| | |
|---|---|
| 5′-GTTCTGTCATATTTCAGTGAATGCGAGTCCGTC | (SEQ ID |
| TAAGAGAGTAGTACAGCAAGAGTGTCTA-3′ | NO: 5) |
| 5′-GCTGTGCAATATTTCAGTGAATGCGAGTCCGTC | (SEQ ID |
| TAAGAGAGTAGTACAGCAAGAGCGTCGA-3′ | NO: 6) |

In embodiments, the invention provides an oligonucleotide comprising a sequence of any one of SEQ ID NOs:5, 6, and 16. In embodiments, the invention provides an oligonucleotide consisting of a sequence of any one of SEQ ID NOs:5, 6, and 16.

In embodiments, the template oligonucleotide comprises one or more connector oligonucleotides, wherein the one or more connector oligonucleotides are capable of being ligated to form a circular template. In embodiments, the 5′ and 3′ ends of the template oligonucleotide are capable of hybridizing to first and second regions of the nucleic acid primer. In embodiments, the template oligonucleotide is a circular oligonucleotide. In embodiments, the template oligonucleotide is a circular oligonucleotide, and the method comprises hybridizing the circular oligonucleotide to the nucleic acid primer to form the second complex as described herein. In embodiments, the template oligonucleotide is a linear oligonucleotide, wherein the 5′ and 3′ ends of the linear oligonucleotide are capable of being ligated to form a circular oligonucleotide. In embodiments, the 5′ and 3′ ends of the linear oligonucleotide are ligated following hybridization of the 5′ and 3′ ends of the template oligonucleotide to the first and second regions of the nucleic acid primer. In embodiments, the template oligonucleotide is a linear oligonucleotide, and the method comprises ligating the 5′ and 3′ ends of the linear oligonucleotide prior to, during, or after hybridization of the nucleic acid primer to the template oligonucleotide, thereby forming a circular template.

In embodiments, the nucleic acid primer is hybridized to the template oligonucleotide to form a second complex, and the nucleic acid primer is extended by PCR. In embodiments, the nucleic acid primer is hybridized to the template oligonucleotide to form a second complex, and the nucleic acid primer is extended by NEAR. In embodiments, the template oligonucleotide is a circular oligonucleotide, the nucleic acid primer is hybridized to the circular oligonucleotide, and the nucleic acid primer is extended by RCA. In embodiments, the nucleic acid primer is hybridized to the template oligonucleotide, the template oligonucleotide is ligated to form a circular template, and the nucleic acid primer is extended by RCA. In embodiments, the extending, e.g., PCR, NEAR, or RCA, is performed at about 15° C. to about 35° C., or about 18° C. to about 30° C., or about 20° C. to about 27° C. In embodiments, the extending, e.g., PCR, NEAR, or RCA, is performed for about 5 to about 120 minutes, or about 5 to about 90 minutes, or about 45 to about 90 minutes, or about 30 to about 60 minutes, or about 15 to about 30 minutes, or about 10 to about 20 minutes, or about 5 to about 10 minutes. In embodiments, the nucleic acid primer is extended to form an extended oligonucleotide.

### Polymerase

In embodiments, the nucleic acid primer is extended by a polymerase. In embodiments, the polymerase is capable of performing PCR, NEAR, or an isothermal amplification method such as SDA, HDA, or RCA. In embodiments, the polymerase is capable of performing SDA, i.e., displacing downstream DNA encountered during synthesis. In embodiments, the polymerase is capable of multiple displacement amplification (MDA). Non-limiting examples of polymerases that can perform PCR, NEAR, SDA, HDA, and/or RCA include Taq polymerase, Vent polymerase, T4 polymerase, T7 polymerase, Phi29 polymerase, Bst polymerase, polymerases from Bacillus bacteriophages Nf, Karezi, and BeachBum (also referred to herein as “Nf polymerase,” “Karezi polymerase” and “BeachBum polymerase,” respectively), and polymerases from other Phi29-like bacteriophages, e.g., as described in Stanton et al., Viruses 13:1557 (2021).

In embodiments, the polymerase comprises strand-displacement activity, referred to herein as an “SD polymerase.” In embodiments, the SD polymerase comprises an amino acid sequence of at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a DNA polymerase from a bacteriophage. In embodiments, the bacteriophage is Phi29, Nf, Karezi, or BeachBum. In embodiments, the SD polymerase comprises an amino acid sequence of at least 80% sequence identity to a DNA polymerase from a bacteriophage, wherein the bacteriophage is Phi29, Nf, Karezi, or BeachBum. In embodiments, the SD polymerase comprises an amino acid sequence of at least 90% sequence identity to a DNA polymerase a bacteriophage, wherein the bacteriophage is Phi29, Nf, Karezi, or BeachBum. In embodiments, the SD polymerase is a DNA polymerase a bacteriophage, wherein the bacteriophage is Phi29, Nf, Karezi, or BeachBum.

### Template Cleavage

In embodiments, the method further comprises terminating the extending of the nucleic acid primer by cleaving the template oligonucleotide. It was surprisingly discovered that cleavage of the template oligonucleotide results in a more stable assay end point, which provides numerous advantages, including, e.g.: allowing the assay to be performed without requiring strict assay timing and/or temperature conditions; shortening the amplification time while improving reproducibility of the amplification, e.g., among multiple wells of an assay plate; allowing an optimal assay signal to be determined; and controlling background signal. An assay that can be performed without strict timing and/or temperature requirements provides valuable flexibility, e.g., in situations where assay plates are batch processed (for example in non-instrumented point-of-care (POC) devices and automated instruments) and may not allow for optimal assay timing, e.g., due to scheduling constraints.

In embodiments, the cleaving comprises contacting a nuclease with the template oligonucleotide. In embodiments, the template oligonucleotide is contacted with the nuclease and the polymerase simultaneously or substantially simultaneously. It was surprisingly discovered that fine-tuning the ratio of polymerase and nuclease within the same reaction allows for a more robust assay procedure. The inclusion of a nuclease with the polymerase allows the two enzymes to function together and achieve the desired result of reducing the temperature and time sensitivity of the reaction, thereby improving assay robustness. The fine-tuning of the enzyme activities, e.g., the polymerase amplification of the template and the nuclease cutting and preventing further polymerase activity, optimized assay robustness. When these two enzyme activities are suitably matched, the reaction is controlled by the balance between the two enzyme activities within the reaction, i.e., the polymerase amplification of the template oligonucleotide and nuclease to prevent further amplification of the template oligonucleotide by the polymerase. This balance of opposing activities, defined by the concentrations of the polymerase and nuclease, results in a reaction in which the amount of extended oligonucleotides formed is not as sensitive to changes in temperature and time, allowing for the reaction to be substantially buffered from the influence of temperature and/or time. In embodiments, the reaction is performed at about 20° C. to about 27° C. while maintaining a substantially consistent assay signal. In embodiments, the use of both the polymerase and the nuclease allows the assay to be performed without requiring a temperature controlled incubator.

In embodiments, the nuclease specifically cleaves the template oligonucleotide. In embodiments, the nuclease does not cleave the nucleic acid primer or the extended oligonucleotide. In embodiments, the nuclease specifically cleaves double-stranded oligonucleotides, e.g., double-stranded DNA, double-stranded RNA, or a double-stranded DNA/RNA hybrid. In embodiments, the double-stranded DNA, double-stranded RNA, or double-stranded DNA/RNA hybrid comprises the template oligonucleotide and the nucleic acid primer hybridized thereto.

In embodiments, each of the template oligonucleotide and the nucleic acid primer comprises single-stranded DNA, which form a double-stranded DNA oligonucleotide upon hybridization. In embodiments, the nuclease specifically cleaves a double-stranded oligonucleotide that is formed by hybridization of the template oligonucleotide to the nucleic acid primer. In embodiments, the nuclease is a restriction endonuclease (also known as a restriction enzyme). Non-limiting examples of restriction endonucleases that may be used include the restriction enzyme as shown in Table 1. In embodiments, the nuclease is DdeI, AluI, HpaII, ApoI, DpnI, DpnII, ScrFI, MboI, MluCI, AseI, TaqI, TspRI, or a combination thereof.

In embodiments, one of the template oligonucleotide or the nucleic acid primer comprises single-stranded DNA, and the other comprises single-stranded RNA, which form a double-stranded DNA/RNA hybrid upon hybridization. In embodiments, the nuclease specifically cleaves a DNA/RNA hybrid that is formed by hybridization of the template oligonucleotide and the nucleic acid primer. Non-limiting examples of nucleases capable of cleaving a DNA/RNA hybrid include RNase H (including RNase H1, H2, and H3) and the restriction endonucleases AvaII, AvrII, BanI, Sau3AI, BstNI, NciI, MvaI, BcnI, and MspI. In embodiments, the nuclease is an endoribonuclease. Non-limiting examples of endoribonucleases comprise RNase III, RNase A, RNase T1, RNase T2, and RNase H. In embodiments, the nuclease is RNase H2.

In embodiments, the template oligonucleotide comprises a DNA damage indicator, and the nuclease comprises an excision enzyme that specifically binds to the DNA damage indicator and cleaves the template oligonucleotide. In embodiments, the DNA damage indicator is not present in the nucleic acid primer or the extended oligonucleotide, and the nuclease does not bind to or cleave the nucleic acid primer or the extended oligonucleotide.

In embodiments, the DNA damage indicator comprises a nucleobase that is typically not present in DNA, and the nuclease cleaves DNA that contains such a nucleobase. In embodiments, the DNA damage indicator comprises an uracil base, and the nuclease comprises uracil-N-glycosylase (UNG). UNG may leave an abasic site at the point of cleavage. In embodiments, the cleaving further comprises providing an abasic site endonuclease. In embodiments, the abasic site endonuclease comprises uracil-DNA glycosylase (UDG), apurinic/apyrimidinic (AP) endonuclease 1 (APE1), Endonuclease IV, or a combination thereof.

In embodiments, the DNA damage indicator comprises deoxyinosine, and the nuclease comprises Endonuclease V. In embodiments, the DNA damage indicator comprises a damaged purine, and the nuclease comprises an enzyme that repairs the damaged purine. In embodiments, the damaged purine comprises 8-oxoguanine (8oxoG), and the nuclease comprises formamidopyrimidine DNA glycosylase (Fpg).

As discussed herein, a method that comprises terminating the extending, e.g., by cleaving the template oligonucleotide, provides several advantages over a method that does not comprise the terminating. In embodiments, an assay signal range produced by the method that comprises the terminating is more stable and less prone to variations based on assay temperature and/or extension time, as compared to a method that does not comprise the terminating. As used herein, an “assay signal range” refers to the ratio of the maximum signal to the minimum signal of assays performed at the indicated conditions, e.g., assay temperature range from 20° C. to 30° C. or extension time range from about 5 minutes to about 90 minutes.

In embodiments, a method that comprises terminating the extending as described herein is less sensitive to variations in assay temperature as compared to a method that does not comprise the terminating. In embodiments, the extending comprises RCA, and the terminating comprises cleaving the template oligonucleotide as described herein, e.g., with a nuclease described herein. In embodiments, the RCA is performed at about 15° C. to about 35° C., or about 18° C. to about 30° C., or about 20° C. to about 27° C. In embodiments, an assay signal range produced by the method does not substantially vary when the method is performed at about 20° C. to about 21° C., 22° C., 23° C., 24° C., 25° C., 26° C., 27° C., 28° C., 29° C., or 30° C.

In embodiments, an assay signal range produced by the method described herein does not vary by more than 5-fold over the assay temperature range of about 17° C. to about 30° C., or about 18° C. to about 29° C., or about 19° C. to about 28° C., or about 20° C. to about 27° C. In embodiments, an assay signal range produced by the method does not vary by more than 4-fold over the assay temperature range of about 17° C. to about 30° C., or about 18° C. to about 29° C., or about 19° C. to about 28° C., or about 20° C. to about 27° C. In embodiments, an assay signal range produced by the method does not vary by more than 4-fold over the assay temperature range of about 17° C. to about 30° C., or about 18° C. to about 29° C., or about 19° C. to about 28° C., or about 20° C. to about 27° C. In embodiments, an assay signal range produced by the method does not vary by more than 3-fold over the assay temperature range of about 17° C. to about 30° C., or about 18° C. to about 29° C., or about 19° C. to about 28° C., or about 20° C. to about 27° C. In embodiments, an assay signal range produced by the method does not vary by more than 1.5-fold over the assay temperature range of about 17° C. to about 30° C., or about 18° C. to about 29° C., or about 19° C. to about 28° C., or about 20° C. to about 27° C.

In embodiments, an assay signal range produced by the method described herein does not vary by more than ±50% over the assay temperature range of about 17° C. to about 30° C., or about 18° C. to about 29° C., or about 19° C. to about 28° C., or about 20° C. to about 27° C. In embodiments, an assay signal range produced by the method does not vary by more than ±40% over the assay temperature range of about 17° C. to about 30° C., or about 18° C. to about 29° C., or about 19° C. to about 28° C., or about 20° C. to about 27° C. In embodiments, an assay signal range produced by the method does not vary by more than ±30% over the assay temperature range of about 17° C. to about 30° C., or about 18° C. to about 29° C., or about 19° C. to about 28° C., or about 20° C. to about 27° C. In embodiments, an assay signal range produced by the method does not vary by more than ±20% over the assay temperature range of about 17° C. to about 30° C., or about 18° C. to about 29° C., or about 19° C. to about 28° C., or about 20° C. to about 27° C. In embodiments, an assay signal range produced by the method does not vary by more than ±10% over the assay temperature range of about 17° C. to about 30° C., or about 18° C. to about 29° C., or about 19° C. to about 28° C., or about 20° C. to about 27° C.

In embodiments, a method as described herein that comprises terminating the extending is less sensitive to variations in extension time as compared to a method that does not comprise the terminating. In embodiments, the extending comprises RCA, and the terminating comprises cleaving the template oligonucleotide as described herein. In embodiments, the extending comprises RCA, and the terminating comprises cleaving the template oligonucleotide as described herein, e.g., with a nuclease described herein. In embodiments, the RCA is performed at about 15° C. to about 35° C., or about 18° C. to about 30° C., or about 20° C. to about 27° C. In embodiments, an assay signal range produced by the method does not substantially vary when the method is performed with an extension time range of about 5 minutes to about 150 minutes, or about 10 minutes to about 120 minutes, or about 15 minutes to about 90 minutes, or about 20 minutes to about 60 minutes, or about 30 minutes to about 45 minutes, or about 5 minutes, about 10 minutes, about 15 minutes, about 20 minutes, about 25 minutes, about 30 minutes, about 35 minutes, about 40 minutes about 45 minutes, about 50 minutes, about 55 minutes, about 60 minutes, about 75 minutes, about 90 minutes, about 105 minutes, or about 120 minutes.

In embodiments, an assay signal range produced by the method described herein does not vary by more than 5-fold, more than 4-fold, more than 3-fold, or more than 2-fold over the extension time range of about 5 to about 120 minutes. In embodiments, an assay signal range produced by the method does not vary by more than 5-fold, more than 4-fold, more than 3-fold, or more than 2-fold over the extension time range of about 30 to about 120 minutes. In embodiments, an assay signal range produced by the method does not vary by more than 5-fold, more than 4-fold, more than 3-fold, or more than 2-fold over the extension time range of about 20 to about 90 minutes. In embodiments, an assay signal range produced by the method does not vary by more than 5-fold, more than 4-fold, more than 3-fold, or more than 2-fold over the extension time range of about 10 to about 45 minutes. In embodiments, an assay signal range produced by the method does not vary by more than 5-fold, more than 4-fold, more than 3-fold, or more than 2-fold over the extension time range of about 5 to about 30 minutes. In embodiments, an assay signal range produced by the method does not vary by more than 5-fold, more than 4-fold, more than 3-fold, or more than 2-fold over the extension time range of 5 to about 15 minutes.

In embodiments, an assay signal range produced by the method described herein does not vary by more than 5-fold, more than 4-fold, more than 3-fold, or more than 2-fold over the extension time range of about 5 to about 90 minutes. In embodiments, an assay signal range produced by the method does not vary by more than 5-fold, more than 4-fold, more than 3-fold, or more than 2-fold over the extension time range of about 45 to about 90 minutes. In embodiments, an assay signal range produced by the method does not vary by more than 5-fold, more than 4-fold, more than 3-fold, or more than 2-fold over the extension time range of about 30 to about 60 minutes. In embodiments, an assay signal range produced by the method does not vary by more than 5-fold, more than 4-fold, more than 3-fold, or more than 2-fold over the extension time range of about 15 to about 30 minutes. In embodiments, an assay signal range produced by the method does not vary by more than 5-fold, more than 4-fold, more than 3-fold, or more than 2-fold over the extension time range of about 10 to about 20 minutes. In embodiments, an assay signal range produced by the method does not vary by more than 5-fold, more than 4-fold, more than 3-fold, or more than 2-fold over the extension time range of 5 to about 10 minutes.

In embodiments, a method that comprises terminating the extending as described herein has a more stable assay end point as compared to a method that does not comprise the terminating. In embodiments, a method that comprises terminating the extending provides a consistent length of extended oligonucleotide. In embodiments, the extending comprises RCA, and the terminating comprises cleaving the template oligonucleotide as described herein. In embodiments, the extending comprises RCA, and the terminating comprises cleaving the template oligonucleotide as described herein, e.g., with a nuclease described herein. In embodiments, the RCA is performed at about 15° C. to about 35° C., or about 18° C. to about 30° C., or about 20° C. to about 27° C. In embodiments, a method that comprises the terminating forms a shorter extended oligonucleotide as compared to a method that does not comprise the terminating.

### Extended Oligonucleotide

In embodiments, the extended oligonucleotide formed by the method described herein is about 100 to about 100000 bases in length. In embodiments, the extended oligonucleotide formed by the method is about 200 to about 75000 bases in length. In embodiments, the extended oligonucleotide formed by the method is about 500 to about 50000 bases in length. In embodiments, the extended oligonucleotide formed by the method is about 700 to about 20000 bases in length. In embodiments, the extended oligonucleotide formed by the method is about 1000 to about 15000 bases in length. In embodiments, the extended oligonucleotide formed by the method is about 2000 to about 10000 bases in length. In embodiments, the extended oligonucleotide formed by the method is about 3000 to about 8000 bases in length. In embodiments, the extended oligonucleotide formed by the method is about 4000 to about 7000 bases in length. In embodiments, the extended oligonucleotide formed by the method is about 5000 to about 6000 bases in length.

In embodiments, the extended oligonucleotide formed by the method described herein is about 100 to about 80000 bases, or about 200 to about 60000 bases, or about 500 to about 50000 bases in length. In embodiments, the extended oligonucleotide formed by the method is about 4000 to about 100000 bases, or about 7500 to about 75000 bases, or about 9000 to about 40000 bases in length. In embodiments, the extended oligonucleotide formed by the method is about 1000 to about 50000 bases, or about 2000 to about 25000 bases, about 3000 to about 13000 bases in length. In embodiments, the extended oligonucleotide formed by the method is about 100 to about 8000 bases, or about 500 to about 6000 bases, or about 1000 to about 4500 bases in length.

In embodiments, the length of the extended oligonucleotide formed by the method described herein is about 1% to about 60%, or about 2% to about 50%, or about 3% to about 45%, or about 4% to about 40%, or about 5% to about 35%, or about 6% to about 32%, or about 8% to about 30%, or about 10% to about 28%, or about 12% to about 25%, or about 15% to about 22%, or about 18% to about 20% of an extended oligonucleotide formed by a method that does not comprise the terminating and is otherwise substantially identical to the method described herein (i.e., a “substantially identical method that does not comprise the terminating”).

In embodiments, the length of the extended oligonucleotide formed by the method described herein is about 1% to about 50%, about 3% to about 40%, or about 5% to about 35% of an extended oligonucleotide formed by a substantially identical method that does not comprise the terminating. In embodiments, the length of the extended oligonucleotide formed by the method described herein is about 2% to about 40%, about 4% to about 37%, or about 6% to about 32% of an extended oligonucleotide formed by a substantially identical method that does not comprise the terminating. In embodiments, the length of the extended oligonucleotide formed by the method described herein is about 1% to about 20%, about 1% to about 15%, or about 1% to about 10% of an extended oligonucleotide formed by a substantially identical method that does not comprise the terminating.

In embodiments, the extended oligonucleotide, formed by polymerase extension of the nucleic acid primer as described herein, comprises a single-stranded oligonucleotide. In embodiments, the extended oligonucleotide is capable of binding to one or more labeled probes, wherein each labeled probe comprises (1) a detection oligonucleotide that is capable of binding to the extended oligonucleotide and (2) a detectable label. In embodiments, the template oligonucleotide comprises a region that comprises a same sequence as a detection oligonucleotide, thereby generating an extended oligonucleotide comprising a sequence that is complementary to the detection oligonucleotide, also referred to herein as a “detection oligonucleotide complement.”

### Detection Oligonucleotide

In embodiments, the extended oligonucleotide binds to the detection oligonucleotide, e.g., via hybridization of complementary oligonucleotides. In embodiments, the detection oligonucleotide comprises a single-stranded oligonucleotide. In embodiments, the detection oligonucleotide comprises RNA, a modified nucleic acid, or a combination thereof.

It was unexpectedly discovered that the detection oligonucleotides provided herein, which are short oligonucleotides of about 3 to about 30 nucleotides in length, about 5 to about 25 nucleotides in length, or about 6 to about 12 nucleotides in length, and that comprise RNA and/or a modified nucleic acid as described herein, had reduced inhibition of polymerases, e.g., SD polymerases described herein. Reduced inhibition of the polymerase allows the detection oligonucleotide to be added to the assay reaction simultaneously or substantially simultaneously as the polymerase, which reduces assay complexity. Additional advantages of shorter detection oligonucleotides include, e.g., that multiple (e.g., at least 2 or at least 3) detection oligonucleotides are capable of binding to an extended oligonucleotide described herein, which increases assay signal; and that shorter oligonucleotides require lower cost and reduced effort to produce and validate as compared to conventional detection oligonucleotides. The detection oligonucleotides provided herein produced higher assay signal even when provided at a lower concentration than conventional detection oligonucleotides, which are described in WO 2014/165061; WO 2014/160192; and WO 2015/175856. In embodiments, incorporation of RNA and/or modified nucleic acids into the short detection oligonucleotides described herein significantly reduces assay background signal. In embodiments, a detection oligonucleotide comprising RNA and/or a modified nucleic acid has higher binding affinity to the extended oligonucleotide, and therefore reduced non-specific binding and less background signal, as compared to a detection oligonucleotide of the same length and that does not comprise any RNA or modified nucleic acids. It was further unexpectedly discovered that RNA oligonucleotides can be used as detection oligonucleotides, which was previously expected to be unfeasible due to instability of RNA and its susceptibility to RNase. It was therefore surprising that RNA-based detection oligonucleotides provided comparable performance in the methods of the present invention as DNA-based detection oligonucleotides without requiring additional measures to maintain an RNase-free environment. Modified nucleic acids are further described herein.

In embodiments, the detection oligonucleotide comprises RNA. In embodiments, the detection oligonucleotide consists of RNA. In embodiments, the detection oligonucleotide comprises a modified nucleic acid. In embodiments, the detection oligonucleotide consists of modified nucleic acids. In embodiments, the detection oligonucleotide comprises a combination of RNA and a modified nucleic acid. In embodiments, the modified nucleic acid is a modified RNA nucleic acid. In embodiments, the modified nucleic acid is a modified DNA nucleic acid.

In embodiments, the modified nucleic acid comprises a peptide nucleic acid (PNA), a locked nucleic acid (LNA), a bridged nucleic acid (BNA), a nucleoside comprising a 2′ modification, or a combination thereof. In embodiments, the modified nucleic acid comprises a PNA, an LNA, a BNA, a nucleoside comprising a 2′ modification, or a combination thereof. In embodiments, the detection oligonucleotide comprises a single modified nucleic acid, e.g., a PNA monomer, LNA monomer, BNA monomer, or a single nucleoside comprising a 2′ modification. In embodiments, the detection oligonucleotide comprises more than one modified nucleic acids, e.g., more than one PNA, LNA, BNA monomers and/or nucleosides comprising a 2′ modification.

In embodiments, the detection oligonucleotide comprises one or more PNAs. In embodiments, the detection oligonucleotide comprises one or more LNAs. In embodiments, the detection oligonucleotide comprises one or more BNAs. PNAs, LNAs, and BNAs are further described herein.

In embodiments, the detection oligonucleotide comprises one or more nucleosides comprising a 2′ modification, also referred to herein as “2′ modified nucleoside.” In embodiments, the 2′ modified nucleoside comprises a 2′-O-methyl modification (2′-OMe), a 2′-O-methoxyethyl modification (2′-MOE), a 2′-deoxy-2′-fluoro modification (2′-F), a 2′-hydroxyl modification (2′-OH), or a combination thereof. See, e.g., Duffy et al., BMC Biology 18:112 (2020).

In embodiments, the detection oligonucleotide comprises a modified nucleic acid comprising a backbone modification, e.g., in the phosphate backbone of one or more nucleotides. Exemplary backbone modifications include, but are not limited to, replacement of the phosphate with a phosphorothioate, boranophosphate, methylphosphonate, phosphoramidate (e.g., morpholino phosphoramidate and mesyl phosphoramidate), phosphoramidite, or 3′-O-phosphopropylamino. See, e.g., Wickstrom et al., Adv Drug Deliver Rev 87:25-34 (2015).

In embodiments, the detection oligonucleotide consists of modified nucleic acids, i.e., wherein each nucleotide of the detection oligonucleotide comprises a modified nucleic acid as described herein. In embodiments, each modified nucleic acid of the detection oligonucleotide comprises a PNA, an LNA, a BNA, a nucleoside comprising a 2′-modification, or combination thereof. In embodiments, a detection oligonucleotide that consists of modified nucleic acids has reduced non-specific binding and lower background signal as compared to a detection oligonucleotide that comprises only unmodified nucleic acids or that comprises a combination of modified and unmodified nucleic acids.

In embodiments, the detection oligonucleotide is about 3 to about 30 nucleotides in length, or about 4 to about 25 nucleotides in length, or about 4 to about 20 nucleotides in length, or about 5 to about 18 nucleotides in length, or about 6 to about 15 nucleotides in length, or about 8 to about 12 nucleotides in length. In embodiments, the detection oligonucleotide is about 4, about 5, about 6, about 7, about 8, about 9, about 10, about 11, about 12, about 13, about 14, about 15, about 16, about 17, about 18, about 19, or about 20 nucleotides in length. As described herein, the detection oligonucleotides provided herein are sufficiently short such that multiple detection oligonucleotides, e.g., at least 2, at least 3, at least 4, at least 5, at least 6, at least 7, at least 8, at least 9, or at least 18 detection oligonucleotides, are capable of binding to an extended oligonucleotide described herein. Further, the detection oligonucleotides provided herein are sufficiently short such that polymerase activity, e.g., of a SD polymerase described herein, is substantially uninhibited in the presence of the detection oligonucleotide. As used herein, the term “substantially” when referring to enzyme activity under different conditions (e.g., presence or absence of the detection oligonucleotide) means that the enzyme activity does not vary (increase or decrease) by more than 20%, more than 15%, more than 10%, more than 5%, or more than 1% under the different conditions.

In embodiments, the method described herein comprises simultaneously or substantially simultaneously contacting the nucleic acid primer with (i) a polymerase and (ii) a labeled probe comprising the detection oligonucleotide described herein, wherein the polymerase extends the nucleic acid primer to form an extended oligonucleotide that binds the detection oligonucleotide of the labeled probe, and wherein polymerase activity of the polymerase is substantially uninhibited by the detection oligonucleotide. In some embodiments, activity of the polymerase remains substantially the same, e.g., does not vary by more than 20%, more than 15%, more than 10%, more than 5%, or more than 1% in the presence of the detection oligonucleotide as compared to activity of the polymerase under the same conditions except without the detection oligonucleotide. In embodiments, the polymerase is an SD polymerase as described herein. In embodiments, the SD polymerase comprises an amino acid sequence of at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a DNA polymerase from a bacteriophage. In embodiments, the bacteriophage is Phi29, Nf, Karezi, or BeachBum.

In embodiments, the detection oligonucleotide comprises or consists of a sequence as shown in Table 4. As used in nucleic acid sequences herein, a lowercase “m” before a nucleobase denotes a 2′-O-methyl modification in that nucleobase. As used in nucleic acid sequences herein, a “+” before a nucleobase denotes an LNA.

TABLE 4
Exemplary detection oligonucleotide sequences

| | |
|---|---|
| mG+A+G+T+C+CmGmUmCmU | SEQ ID NO: 7 |
| C+A+G+T+G+AA+TGC | SEQ ID NO: 8 |
| G+A+G+T+C+C+GTCT | SEQ ID NO: 9 |
| G+A+G+T+C+CGTCT | SEQ ID NO: 10 |

In embodiments, the invention provides an oligonucleotide comprising a sequence of any one of SEQ ID NOs:7-10 and 12-15. In embodiments, the invention provides an oligonucleotide consisting of a sequence of any one of SEQ ID NOs:7-10 and 12-15.

### Detectable Label

In embodiments, the labeled probe comprises a detection oligonucleotide as described herein and a detectable label. In embodiments, the labeled probe comprises more than one detectable label. In embodiments, the labeled probe comprises 1, 2, 3, 4, 5, 6, 7, 8, 9, 10, or more than 10 detectable labels. In embodiments, one or more detectable labels are linked to the detection oligonucleotide, e.g., to a 5′ end or a 3′ end of the detection oligonucleotide. Methods of linking detectable labels to an oligonucleotide, e.g., a detection oligonucleotide as described herein, are known to one of ordinary skill in the art and described, e.g., in WO 2020/180645.

In embodiments, the detectable label is capable of being detected by light scattering, optical absorbance, fluorescence, chemiluminescence, electrochemiluminescence (ECL), bioluminescence, phosphorescence, radioactivity, magnetic field, or combination thereof. In embodiments, the method is a multiplexed method capable of detecting at least two unique analytes, and the detectable label for each unique analyte comprises a distinct detectable signal. Multiplexed methods are further described herein. In embodiments, the detectable label comprises a fluorescent label, and a distinct fluorescence signature (e.g., fluorescent wavelength and/or intensity) is associated with each unique analyte, thereby allowing the unique analytes to be distinguished from each other.

In embodiments, the detectable label is an ECL label. In embodiments, the labeled probe comprises about 1 to 10, or about 2 to 5, or about 3 to 4 ECL labels. In embodiments, the labeled probe comprises three ECL labels. In embodiments, the ECL label comprises an electrochemiluminescent organometallic complex of ruthenium, osmium, iridium, rhenium, and/or a lanthanide metal. In embodiments, the ECL label comprises an organometallic complex comprising at least one substituted bipyridine ligand, wherein the substituted bipyridine ligand comprises at least one sulfonate group. In embodiments, the ECL label comprises an organometallic complex comprising at least two substituted bipyridine ligands, wherein each substituted bipyridine ligand comprises at least one sulfonate group. Exemplary ECL labels can be found in U.S. Pat. Nos. 5,714,089; 6,136,268; 6,316,607; 6,468,741; 6,479,233; 6,808,939; and 9,499,573.

In embodiments, the method comprises detecting the detectable label. In embodiments, the detecting comprises measuring light scattering, optical absorbance, fluorescence, chemiluminescence, electrochemiluminescence (ECL), bioluminescence, phosphorescence, radioactivity, magnetic field, or combination thereof. In embodiments, the measured amount of detectable label is used to determine the amount of analyte present in the sample.

In embodiments, the method comprises detecting the amount of detectable label present in the second complex on a surface. In embodiments, the second comprises the analyte, the detection reagent bound to the analyte, the extended oligonucleotide formed from the nucleic acid primer on the detection reagent, the labeled probe bound to the extended oligonucleotide. In embodiments, the surface comprises a particle. In embodiments, the surface comprises a well of multi-well plate. Surfaces are further described herein. In embodiments, the surface comprises a particle, the detectable label comprises a fluorescence label, and the method comprises detecting the fluorescence label by a particle analysis method. Methods of analyzing particles, e.g., comprising a fluorescence label, are known to one of ordinary skill in the art. In embodiments, the particle analysis method comprises detecting the detectable label by flow cytometry. In embodiments, the particle analysis method comprises immobilizing the particles on a particle collection surface, and detecting the detectable label on the immobilized particles. In embodiments, the particles are immobilized in a single layer on the particle collection surface. In embodiments, the particle collection surface comprises an electrode. In embodiments, the particle collection surface comprises a slide (e.g., a microscope slide), a chip, or a flow cell. In embodiments, the immobilized particles are detected by imaging the particle collection surface and determining the number of particles comprising the detectable label, e.g., fluorescence label. In embodiments, the detectable label comprises an ECL label, and the surface comprises an electrode. In embodiments, the electrode comprises a carbon ink electrode. In embodiments, the detecting comprises applying a voltage waveform (e.g., a potential) to the electrode to general an ECL signal. In embodiments, the surface comprises a particle, and the method comprises collecting the particle on an electrode and applying a voltage waveform (e.g., a potential) to the electrode to generate an ECL signal.

### Anchoring Reagent

In embodiments, the second complex, which is formed by hybridization of the template oligonucleotide to the nucleic acid primer, is bound to a surface. In embodiments, the surface comprises an anchoring reagent. In embodiments, the anchoring reagent binds to the extended oligonucleotide formed by extending the nucleic acid primer. In embodiments, the second complex is bound to the surface via binding of the extended oligonucleotide to the anchoring reagent on the surface. In embodiments, binding of the extended oligonucleotide to the anchoring reagent stabilizes the second complex on the surface. In embodiments, binding of the extended oligonucleotide to the anchoring reagent facilitates binding of the labeled probe to the extended oligonucleotide and improves assay signal. Anchoring reagents are further described in, e.g., WO 2014/165061; WO 2014/160192; WO 2015/175856; and WO 2020/180645.

In embodiments, the template oligonucleotide comprises a region comprising a same sequence as an anchoring oligonucleotide, thereby generating an extended oligonucleotide comprising a sequence that is complementary to the anchoring oligonucleotide, also referred to herein as an “anchoring oligonucleotide complement.” In embodiments, the template oligonucleotide comprises a first region comprising a same sequence as a detection oligonucleotide and a second region comprising a same sequence as an anchoring oligonucleotide, thereby generating an extended oligonucleotide comprising a first sequence that is complementary to the detection oligonucleotide and a second sequence that is complementary to the anchoring oligonucleotide. In embodiments, the extended oligonucleotide binds to the anchoring reagent prior to or at the same time as binding to the labeled probe as described herein. In embodiments, the extended oligonucleotide binds to the anchoring reagent prior to being contacted with the labeled probes. In embodiments, the extended oligonucleotide is contacted simultaneously or substantially simultaneously with the anchoring reagent and the labeled probe.

In embodiments, the anchoring reagent comprises an oligonucleotide, aptamer, aptamer ligand, antibody, antigen, ligand, receptor, hapten, epitope, or mimotope. In embodiments, the anchoring reagent comprises an aptamer ligand, and the extended oligonucleotide comprises an aptamer. In embodiments, the anchoring reagent comprises an oligonucleotide-binding protein, and the extended oligonucleotide comprises a sequence capable of binding to the protein. In embodiments, the anchoring reagent comprises an anchoring oligonucleotide. In embodiments, the anchoring oligonucleotide comprises a single stranded oligonucleotide. In embodiments, the anchoring oligonucleotide comprises a double stranded oligonucleotide.

In embodiments, binding the extended oligonucleotide to the anchoring reagent comprises forming a triple helix between the anchoring oligonucleotide and the extended oligonucleotide. In embodiments, binding the extended oligonucleotide to the anchoring reagent comprises denaturing the extended oligonucleotide to expose a single stranded oligonucleotide region prior to the binding. In embodiments, binding the extended oligonucleotide to the anchoring reagent comprises exposing the extended oligonucleotide to helicase activity prior to the binding. In embodiments, binding the extended oligonucleotide to the anchoring reagent comprises exposing the extended oligonucleotide to nuclease treatment prior to the binding. In embodiments, the extended oligonucleotide comprises one or more hapten-modified bases, and the anchoring reagent comprises one or more antibodies specific for the hapten. In embodiments, the hapten of the hapten-modified base comprises digoxigenin, and the anchoring reagent comprises an anti-digoxigenin antibody. In embodiments, the extended oligonucleotide comprises one or more ligand-modified bases, and the anchoring reagent comprises one or more receptors specific for the ligand. In embodiments, the anchoring reagent comprises an anchoring oligonucleotide, wherein the anchoring oligonucleotide and the extended oligonucleotide comprise complementary oligonucleotides, and binding the extended oligonucleotide to the anchoring reagent comprises hybridization of the complementary oligonucleotides.

In embodiments, the anchoring reagents provided herein comprise short oligonucleotides of about 3 to about 30 nucleotides in length. It was unexpectedly discovered that short anchoring oligonucleotides had improved stability during assay wash steps. However, shorter anchoring oligonucleotides may increase sample matrix interference effects. The present inventors further discovered that a combination of the short anchoring oligonucleotide length and modified nucleic acids provided improved assay stability without increasing sample matrix interference. In embodiments, an anchoring oligonucleotide comprising modified nucleic acids has reduced sample matrix interference and reduced background signal as compared to an anchoring oligonucleotide that does not comprise any modified nucleic acids.

In embodiments, the anchoring oligonucleotide comprises a modified nucleic acid. Modified nucleic acids are further described herein. In embodiments, the modified nucleic acid comprises a modified base, modified sugar, and/or modified backbone. In embodiments, the modified nucleic acid comprises a PNA, an LNA, a BNA, a nucleoside comprising a 2′ modification, or a combination thereof. In embodiments, the nucleoside comprising a 2′ modification comprises a 2′-OMe, a 2′-MOE, a 2′-F, a 2′-OH, or combination thereof. In embodiments, the modified nucleic acid comprises a backbone modification, e.g., replacement of the phosphate backbone with a phosphorothioate, boranophosphate, methylphosphonate, phosphoramidate (e.g., morpholino phosphoramidate and mesyl phosphoramidate), phosphoramidite, 3′-O-phosphopropylamino, or combination thereof.

In embodiments, the anchoring oligonucleotide comprises a single modified nucleic acid, e.g., a PNA monomer, LNA monomer, BNA monomer, or a single nucleoside comprising a 2′ modification. In embodiments, the anchoring oligonucleotide comprises more than one modified nucleic acids, e.g., more than one PNA, LNA, BNA monomers and/or nucleosides comprising a 2′ modification.

In embodiments, the anchoring oligonucleotide consists of modified nucleic acids, i.e., wherein each nucleotide of the anchoring oligonucleotide comprises a modified nucleic acid as described herein. In embodiments, each modified nucleic acid of the anchoring oligonucleotide comprises a PNA, an LNA, a BNA, a nucleoside comprising a 2′-modification, or combination thereof. In embodiments, an anchoring oligonucleotide that consists of modified nucleic acids has reduced non-specific binding and lower background signal as compared to an anchoring oligonucleotide that comprises only unmodified nucleic acids or that comprises a combination of modified and unmodified nucleic acids.

In embodiments, the anchoring oligonucleotide is about 3 to about 30 nucleotides in length, or about 4 to about 25 nucleotides in length, or about 4 to about 20 nucleotides in length, or about 5 to about 18 nucleotides in length, or about 6 to about 15 nucleotides in length, or about 8 to about 12 nucleotides in length. In embodiments, the anchoring oligonucleotide is about 4, about 5, about 6, about 7, about 8, about 9, about 10, about 11, about 12, about 13, about 14, about 15, about 16, about 17, about 18, about 19, or about 20 nucleotides in length. In embodiments, the anchoring oligonucleotides provided herein are sufficiently short such that the extended oligonucleotide binds to the anchoring oligonucleotide with at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, or at least 10-fold higher affinity as compared to binding an anchoring oligonucleotide that is longer than about 30 nucleotides in length. In embodiments, an anchoring oligonucleotide of about 4 to about 20 nucleotides in length and comprising a modified nucleic acid as described herein, provides an equivalent assay performance when present at the same concentration on a surface as an anchoring oligonucleotide that is at least or about 25 nucleotides in length and that does not comprise any modified nucleic acids.

In embodiments, the anchoring oligonucleotide comprises or consists of the following sequence: mU+AmGmUmA+C+AmGmC (SEQ ID NO:11), wherein the lowercase “m” before a nucleobase denotes a 2′-O-methyl modification in that nucleobase, and the “+” before a nucleobase denotes an LNA as described herein. In embodiments, In embodiments, the invention provides an oligonucleotide comprising a sequence of any one of SEQ ID NOs:11, 17, and 20-37. In embodiments, the invention provides an oligonucleotide consisting of a sequence of any one of SEQ ID NOs:11, 17, and 20-37.

In embodiments, the anchoring reagent is immobilized on the surface prior to step (a) of the method described herein. In embodiments, the anchoring reagent is immobilized on the surface prior to extending of the nucleic acid primer to form the extended oligonucleotide as described herein. In embodiments, the anchoring reagent is immobilized on the surface prior to binding the extended oligonucleotide to the labeled probe as described herein. In embodiments, the anchoring reagent is immobilized on the surface prior to detecting the detectable label of the labeled probe bound to the extended oligonucleotide. Methods and timing of immobilizing anchoring reagents on surfaces are further described, e.g., in US 2022/0341923.

In embodiments, the anchoring reagent is directly immobilized on the surface, e.g., covalently immobilized to the surface via a covalent linkage as described herein. In embodiments, the covalent linkage is formed from a reaction between a thiol group on the anchoring reagent and the surface. In embodiments, the anchoring reagent comprises an anchoring oligonucleotide, wherein the anchoring oligonucleotide is directly immobilized on the surface. In embodiments, the anchoring reagent is indirectly immobilized on the surface, e.g., via secondary binding partners as described herein. In embodiments, the anchoring reagent comprises an anchoring oligonucleotide, wherein the anchoring oligonucleotide is indirectly immobilized on the surface. In embodiments, the anchoring reagent is linked to a first binding partner, the surface comprises a second binding partner, and the anchoring reagent is immobilized on the surface via an interaction of the first and second binding partners. In embodiments, the first and second binding partners comprise complementary oligonucleotides, a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the first and second binding partners comprise cross-reactive moieties, e.g., thiol and maleimide or iodoacetamide; aldehyde and hydrazide; or azide and alkyne or cycloalkyne. In embodiments, the first binding partner comprises biotin, and the second binding partner comprises avidin, streptavidin, an anti-biotin antibody, or a combination thereof. In embodiments, the first and second binding partners bind to each other via a bridging agent, which binds both the first and second binding partners. In embodiments, the bridging agent comprises at least two binding sites, wherein each of the first and second binding partners bind to a distinct binding site. In embodiments, the bridging agent comprises streptavidin or avidin, and the first and second binding partners are each biotin.

In embodiments, the anchoring reagent comprises an anchoring oligonucleotide and a first binding partner, wherein the first binding partner is linked to a nucleotide of the anchoring oligonucleotide. In embodiments, the first binding partner is linked to an internal nucleotide of the anchoring oligonucleotide. In embodiments, the first binding partner is positioned at a 5′-end of the anchoring reagent. In embodiments, the first binding partner is positioned at a 3′-end of the anchoring reagent. In embodiments, the first binding partner is linked to a 5′- or 3′-terminal nucleotide of the anchoring oligonucleotide. In embodiments, the anchoring reagent comprises a spacer positioned between the first binding partner and the anchoring oligonucleotide. In embodiments, the spacer comprises a polyethylene glycol (PEG) comprising about 1 to about 50, or about 2 to about 40, or about 3 to about 30, or about 4 to about 20, or about 5 to about 10, or about 1 to about 15, or about 2 to about 10, or about 3 to about 8, or about 4 to about 7, or about 5 to about 6 ethylene glycol units. In embodiments, the spacer comprises a PEG comprising about 1, 2, 3, 4, 5, 6, 7, 8, 9, 10, 11, 12, 13, 14, 15, 16, 17, 18, 19, or 20 ethylene glycol units. In embodiments, the first binding partner is positioned at a 3′-end of the anchoring reagent, and the anchoring reagent comprises a PEG spacer comprising about 1 to about 15, or about 2 to about 10, or about 3 to about 8 ethylene glycol units. In embodiments, the anchoring reagent comprises a 3′-terminal nucleotide that is linked to a first end of a PEG spacer and a first binding partner that is linked to a second end of a PEG spacer, wherein the PEG spacer comprises about 1 to about 15, or about 2 to about 10, or about 3 to about 8 ethylene glycol units.

### First and Second Complexes

In embodiments, the first complex comprising the analyte and the detection reagent is bound to a surface. In embodiments, the first complex further comprises a capture reagent, wherein the capture reagent specifically binds to the analyte and is immobilized to the surface or capable of being immobilized to the surface. In embodiments, the capture reagent is directly immobilized on the surface, e.g., via a covalent linkage between the capture reagent and the surface. In embodiments, the covalent linkage is formed from a reaction between a thiol group on the capture reagent and the surface. In embodiments, the capture reagent is indirectly immobilized on the surface, e.g., via secondary binding partners. In embodiments, the capture reagent is linked to a first binding partner, which binds to a second binding partner that is immobilized on the surface. In embodiments, the first and second binding partners comprise complementary oligonucleotides, a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the first and second binding partners comprise cross-reactive moieties, e.g., thiol and maleimide or iodoacetamide; aldehyde and hydrazide; or azide and alkyne or cycloalkyne. In embodiments, the first binding partner comprises biotin, and the second binding partner comprises avidin, streptavidin, an anti-biotin antibody, or a combination thereof. In embodiments, the first and second binding partners bind to each other via a bridging agent, which binds both the first and second binding partners. In embodiments, the bridging agent comprises at least two binding sites, wherein each of the first and second binding partners bind to a distinct binding site. In embodiments, the bridging agent comprises streptavidin or avidin, and the first and second binding partners are each biotin.

In embodiments, the capture reagent comprises a protein or polypeptide, antibody or antigen-binding fragment thereof, antigen, ligand, receptor, oligonucleotide, hapten, epitope, mimotope, or aptamer. In embodiments, the capture reagent comprises an antibody or a variant thereof, including an antigen/epitope-binding portion thereof, an antibody fragment or derivative, an antibody analogue, an engineered antibody, or a substance that binds to antigens in a similar manner to antibodies. In embodiments, the capture reagent comprises at least one heavy or light chain complementarity determining region (CDR) of an antibody. In embodiments, the capture reagent comprises at least two CDRs from one or more antibodies. In embodiments, the capture reagent comprises an antibody or antigen-binding fragment thereof. In embodiments, the capture reagent comprises an antigen-binding domain that specifically binds to an epitope of the analyte. In embodiments, the capture reagent comprises an oligonucleotide. In embodiments, the analyte comprises an oligonucleotide, and the capture reagent and the analyte comprise complementary oligonucleotides.

In embodiments, each of the capture reagent and the detection reagent comprises an antibody or antigen-binding fragment thereof. In embodiments, each of the capture reagent and the detection comprises an oligonucleotide.

In embodiments, the method of the invention further comprises forming the first complex prior to contacting the first complex with the template oligonucleotide as described herein. In embodiments, the method of the invention further comprises forming the first complex at substantially the same time as contacting the components of the first complex with the template oligonucleotide as described herein. In embodiments, the capture reagent is immobilized on the surface prior to formation of the first complex. In embodiments, the capture reagent is immobilized on the surface following formation of the first complex.

In embodiments, the first complex is formed by contacting a sample comprising the analyte with the detection reagent. In embodiments, the first complex is formed by contacting a sample comprising the analyte with the capture reagent and the detection reagent. In embodiments, the first complex is formed by contacting a sample comprising the analyte with: first, the capture reagent, and second, the detection reagent. In embodiments, the first complex is formed by contacting a sample comprising the analyte with: first, the detection reagent, and second, the capture reagent. In embodiments, the first complex is formed by contacting a sample comprising the analyte with the capture reagent and the detection reagent simultaneously or substantially simultaneously.

In embodiments, the method comprises simultaneously or substantially simultaneously contacting a sample comprising the analyte with a detection reagent and a template oligonucleotide as described herein. In embodiments, the method comprises simultaneously or substantially simultaneously contacting a sample comprising the analyte with a detection reagent, a template oligonucleotide, and a polymerase as described herein. In embodiments, the method comprises simultaneously or substantially simultaneously contacting a sample comprising the analyte with a detection reagent, a template oligonucleotide, a polymerase, and a labeled probe as described herein. In embodiments, the method comprises simultaneously or substantially simultaneously contacting a sample comprising the analyte with a detection reagent, a template oligonucleotide, a polymerase, a labeled probe, and a nuclease as described herein.

In embodiments, the method comprises forming a first complex comprising the analyte and the detection reagent, and contacting the first complex with a template oligonucleotide as described herein. In embodiments, the method comprises forming a first complex comprising the analyte and the detection reagent, and contacting the first complex with a template oligonucleotide and a polymerase simultaneously or substantially simultaneously. In embodiments, the method comprises forming a first complex comprising the analyte and the detection reagent, and contacting the first complex with a template oligonucleotide, a polymerase, and a labeled probe simultaneously or substantially simultaneously. In embodiments, the method comprises forming a first complex comprising the analyte and the detection reagent, and contacting the first complex with a template oligonucleotide, a polymerase, a labeled probe, and a nuclease simultaneously or substantially simultaneously.

In embodiments, the method comprises simultaneously or substantially simultaneously contacting a sample comprising the analyte with the capture reagent, the detection reagent, and the template oligonucleotide as described herein. In embodiments, the method comprises simultaneously or substantially simultaneously contacting a sample comprising the analyte with the capture reagent, the detection reagent, the template oligonucleotide, and a polymerase as described herein. In embodiments, the method comprises simultaneously or substantially simultaneously contacting a sample comprising the analyte with the capture reagent, the detection reagent, the template oligonucleotide, a polymerase, and a labeled probe as described herein. In embodiments, the method comprises simultaneously or substantially simultaneously contacting a sample comprising the analyte with the capture reagent, the detection reagent, the template oligonucleotide, a polymerase, a labeled probe, and a nuclease as described herein.

In embodiments, the method comprises forming a first complex comprising the analyte, the capture reagent, and the detection reagent, and contacting the first complex with a template oligonucleotide as described herein. In embodiments, the method comprises forming a first complex comprising the analyte, the capture reagent, and the detection reagent, and contacting the first complex with a template oligonucleotide and a polymerase simultaneously or substantially simultaneously. In embodiments, the method comprises forming a first complex comprising the analyte, the capture reagent, and the detection reagent, and contacting the first complex with a template oligonucleotide, a polymerase, and a labeled probe simultaneously or substantially simultaneously. In embodiments, the method comprises forming a first complex comprising the analyte, the capture reagent, and the detection reagent, and contacting the first complex with a template oligonucleotide, a polymerase, a labeled probe, and a nuclease simultaneously or substantially simultaneously.

### Surface

In embodiments, the first and/or second complex described herein is bound to a surface, e.g., via a capture reagent and/or an anchoring reagent as described herein. In embodiments, the surface comprises a particle. In some embodiments, the particle comprises a microsphere. In embodiments, the particle comprises a paramagnetic bead. In embodiments, the particle comprises a bead that is capable of being analyzed via flow cytometry. In embodiments, the flow cytometry detects particles comprising a detectable label described herein, e.g., a fluorescence label. In embodiments, the flow cytometry is capable of distinguishing between particles comprising different fluorescence labels (e.g., distinct fluorescence wavelength and/or intensity). In embodiments, the flow cytometry is capable of distinguishing between particles of different sizes. In embodiments, the particle comprises a bead that is capable of being immobilized onto a particle collection surface for detection as described herein, e.g., by imaging. In embodiments, the particles (e.g., beads) are capable of being immobilized in a single layer on the particle collection surface. In embodiments, the immobilizing comprises dropcasting a solution comprising the particles (e.g., beads) onto a particle collection surface, and evaporating the solution to form a thin film comprising the particles on the particle collection surface, or catalyzing gelatin of the solution to immobilize the particles on the particle collection surface, or a combination thereof. In embodiments, the particle collection surface comprises an electrode. In embodiments, the particle collection surface comprises a glass surface. In embodiments, the particle collection surface comprises a slide (e.g., a microscope slide), a chip, or a flow cell. In embodiments, the immobilized particles are detected by imaging the particle collection surface and determining the number of particles comprising the detectable label. In embodiments, the surface comprises a cartridge. In embodiments, the surface comprises a well of multi-well plate. Non-limiting examples of plates include the MSD SECTOR™ and MSD QUICKPLEX® assay plates, e.g., MSD GOLD™ 96-well Small Spot Streptavidin plate.

In embodiments, the surface comprises a plurality of distinct binding domains, and the capture reagent and anchoring reagent are located on two distinct binding domains on the surface. In embodiments, the surface comprises a plurality of distinct binding domains, and the capture reagent and anchoring reagent are located on the same binding domain on the surface. In embodiments, the surface comprises a particle, wherein the capture reagent and the anchoring reagent are located on the same particle. In embodiments, the capture reagent is within about 1 nm to about 500 nm, about 5 nm to about 250 nm, about 10 nm to about 200 nm, or about 15 nm to about 150 nm of the anchoring reagent on the surface. In embodiments, the capture reagent is less than 1 m from the anchoring reagent on the surface. In embodiments, the capture reagent is less than 500 nm from the anchoring reagent on the surface. In embodiments, the capture reagent is less than 200 nm from the anchoring reagent on the surface.

In embodiments, the surface comprises an electrode. In embodiments, the electrode comprises a carbon ink electrode. In embodiments, the detectable label comprises an ECL label. In embodiments, detecting the detectable label comprises applying a voltage waveform (e.g., a potential) to the electrode to general an ECL signal. In embodiments, the surface comprises a particle, and detecting the detectable label comprises collecting the particle on an electrode and applying a voltage waveform (e.g., a potential) to the electrode to generate an ECL signal.

### Multiplexed Methods

In embodiments, the method is a multiplexed method capable of detecting multiple (e.g., at least two) analytes. In embodiments, the multiplexed method detects about 2 to about 15, or about 3 to about 14, or about 4 to about 13, or about 4 to about 12, or about 5 to about 11, or about 6 to about 10, or about 7 to about 9 analytes simultaneously or substantially simultaneously. In embodiments, the multiplexed method comprises repeating one or more method steps to detect the at least 2, e.g., about 2 to about 15, or about 3 to about 14, or about 4 to about 13, or about 4 to about 12, or about 5 to about 11, or about 6 to about 10, or about 7 to about 9 analytes. In embodiments, each of the method steps is performed for each analyte in parallel. Methods of conducting multiplexed assays are further described in, e.g., U.S. Pat. Nos. 10,189,023 and 10,201,812. In embodiments, each unique analyte is associated with a distinct detectable label that comprises a distinct detectable signal. In embodiments, the detectable label comprises a fluorescent label, and a distinct fluorescence signature (e.g., fluorescent wavelength and/or intensity) is associated with each unique analyte, thereby allowing the analytes to be distinguished from each other, e.g., via flow cytometry, and the amount of analyte associated with each fluorescence signature can be determined. Fluorescent dyes with distinct fluorescence signatures are known to one of ordinary skill in the art. In embodiments, each unique analyte is associated with a distinct surface, e.g., particle with a distinct size. In embodiments, the analytes associated with different particle sizes are capable of being separated, e.g., by flow cytometry, and the number of particles of each size is determined, thereby determining the amount of analyte associated with each particle size.

In embodiments, each analyte is present in a distinct first complex. In embodiments, each first complex comprises a distinct analyte and its corresponding detection reagent. In embodiments, each first complex comprises a distinct analyte and its corresponding capture and detection reagents. In embodiments, the surface comprises a plurality of distinct binding domains, and each analyte forms a first complex in a distinct binding domain. In embodiments, the surface comprises a plurality of capture reagents, wherein each capture reagent is immobilized on a distinct binding domain and the surface, and wherein each capture reagent is capable of binding specifically to one of the at least 2, at least 3, at least 4, at least 5, at least 6, at least 7, at least 8, at least 9, or at least 10 analytes. In embodiments, the surface is contacted with the at least 2, at least 3, at least 4, at least 5, at least 6, at least 7, at least 8, at least 9, or at least 10 analytes, wherein each analyte forms a first complex in its corresponding binding domain.

In embodiments, the plurality of distinct binding domains is on a single surface. In embodiments, the surface comprises a multi-well plate, and each binding domain is in a distinct well. In embodiments, the surface comprises a multi-well plate, and each binding domain is in a distinct region of the well. In embodiments, the plurality of distinct binding domains is on one or more surface. In embodiments, the surface comprises a particle, and each binding domain is on a distinct particle. In embodiments, the particles are arranged in a particle array. In embodiments, the particles are coded to allow for identification of specific particles and distinguish between each binding domain. In embodiments, the binding domains are separable from one another, e.g., via flow cytometry. In embodiments, each unique analyte is associated with a distinct detectable label that comprises a distinct detectable signal as described herein. In embodiments, each distinct detectable label comprises a distinct fluorescence signature (e.g., wavelength and/or intensity), thereby allowing the unique analyte on the particle to be identified based on the distinct fluorescence signature by flow cytometry. In embodiments, each unique analyte is associated with a distinct particle size, thereby allowing the unique analyte on the particle to be identified based on the distinct particle size by flow cytometry. In embodiments, the surface is a multi-well plate comprising detachable wells, and each binding domain is in a different well. In embodiments, the surface comprises one or more particles, and each particle is separable from the remaining particles. Methods of separating particles are known in the field and include, e.g., flow cytometry, magnetic separation, affinity separation, and the like.

### Analytes and Samples

In embodiments, the sample is a biological sample. In embodiments, the sample is an environmental sample. In embodiments, the sample is obtained from a human subject. In embodiments, the sample is obtained from an animal subject. In embodiments, the sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions, or an extraction, purification therefrom, or dilution thereof. Further exemplary samples include but are not limited to physiological samples, samples containing suspensions of cells such as mucosal swabs, tissue aspirates, tissue homogenates, cell cultures, and cell culture supernatants. In embodiments, the sample is whole blood, serum, plasma, cerebrospinal fluid, urine, saliva, or an extraction or purification therefrom, or dilution thereof. In embodiments, the sample is serum or plasma. In embodiments, the plasma is in EDTA, heparin, or citrate. Samples may be obtained from a single source described herein, or may contain a mixture from two or more sources.

Analytes that may be measured using the methods of the invention include, but are not limited to, proteins, toxins, nucleic acids, microorganisms, viruses, cells, fungi, spores, carbohydrates, lipids, glycoproteins, lipoproteins, polysaccharides, drugs, hormones, steroids, nutrients, metabolites, and any modified derivative of the above molecules, or any complex comprising one or more of the above molecules or combinations thereof. The level of an analyte of interest in a sample may be indicative of a disease or disease condition or it may simply indicate whether a subject was exposed to that analyte.

In embodiments, the analyte comprises a biomarker. As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility or responsiveness of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2):182-188 (2004); Strimbu et al., Curr Opin HIV AIDS 5(6):463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. Unless specified otherwise, the biomarkers referred to herein encompass human biomarkers. In embodiments, the biomarker comprises an immune response biomarker. In embodiments, the biomarker comprises an antibody or fragment thereof, e.g., an antigen-binding fragment of an antibody.

In embodiments, the analyte comprises an exosome. In embodiments, the sample comprises purified exosomes. Exosomes, also known as extracellular vesicles or EVs, are small membrane vesicles released by most cell types. The release and subsequent uptake of exosomes is a method of cell-to-cell communication and has a role in the regulation of many physiological and pathological processes. Exosomes have been shown to contain a wide variety of signaling molecules including but not limited to surface-bound and cytosolic proteins, lipids, mRNA, and miRNA, and it has been suggested that the identity and concentration of these species in each exosome can be used to deduce its cellular origin and function. Thus, genomic or proteomic profiling of a patient's total exosome population could provide valuable prognostic information for various pathological conditions, including cancers, infectious disease, kidney and liver disease, and traumatic brain injury, among others. In embodiments, the analyte comprises an internal analyte of an exosome, e.g., a cargo protein, a lipid, or a nucleic acid. Detection of exosomes is further described in, e.g., WO 2015/175856; WO 2019/222708; WO 2020/086751; and WO 2022/051481.

### Assay Formats

In embodiments, the methods provided herein are in a competitive assay format. In general, a competitive assay, e.g., a competitive immunoassay or a competitive inhibition assay, an analyte and a competitor compete for binding to a capture and/or detection reagent. In such assays, the analyte is typically indirectly measured by directly measuring the competitor. As used herein, “competitor” refers to a compound capable of binding to the same capture and/or detection reagent as an analyte, such that the capture and/or detection reagent can only bind either the analyte or the competitor, but not both. In embodiments, competitive assays are used to detect and measure analytes that are not capable of binding more than one capture and/or detection reagents, e.g., small molecule analytes or analytes that do not have more than one distinct binding sites. In embodiments, competitive assays are used to detect and measure antibody biomarkers. Examples of competitive immunoassays include those described in U.S. Pat. Nos. 4,235,601; 4,442,204; and 5,028,535.

The methods herein can be conducted in a single assay chamber, such as a single well of an assay plate. The methods herein can also be conducted in an assay chamber of an assay cartridge. The assay modules, e.g., assay plates or assay cartridges, methods and apparatuses for conducting assay measurements suitable for the invention, are described, e.g., in U.S. Pat. Nos. 8,343,526; 9,731,297; 9,921,166; 10,184,884; 10,281,678; 10,272,436; US 2004/0022677; US 2004/0189311; US 2005/0052646; US 2005/0142033; US 2018/0074082; and US 2019/0391170.

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477.

Assay devices consistent with embodiments herein may be employed for, e.g., conducting assays in a multi-well plate format that have one or more of the following desirable attributes: (i) high sensitivity, (ii) large dynamic range, (iii) small size and weight, (iv) array-based multiplexing capability, (v) automated operation; and (vi) ability to handle multiple plates. The apparatus and methods may be used with a variety of assay detection techniques including, but not limited to, techniques measuring one or more detectable signals. Some aspects are suitable for electrochemiluminescence measurements and, in particular, embodiments that are suitable for use with multi-well plates with integrated electrodes (and assay methods using these plates) such as those described in U.S. Pat. Nos. 7,842,246; 7,807,448; and 10,281,678.

### Kits

In embodiments, the invention provides a kit for detecting an analyte, comprising, in one or more vials, containers, or compartments:
(a) a capture reagent that binds the analyte;
(b) a detection reagent that binds the analyte, wherein the detection reagent comprises a nucleic acid primer or is capable of being linked to a nucleic acid primer;
(c) a labeled probe that comprises (1) a detection oligonucleotide and (2) a detectable label; and
(d) a template oligonucleotide that is capable of hybridizing to the nucleic acid primer and that comprises a same sequence as the detection oligonucleotide; wherein the detection reagent comprises a protein or polypeptide, and wherein:
   (i) the detection oligonucleotide comprises RNA, a modified nucleic acid, or a combination thereof;
   (ii) the kit further comprises a nuclease that is capable of cleaving the template oligonucleotide;
   (iii) combination of (i) and (ii);
   (iv) one or both of (i) and (ii), and wherein the kit further comprises an anchoring reagent;
   (v) the kit further comprises an anchoring reagent that comprises an anchoring oligonucleotide, wherein the anchoring oligonucleotide comprises a modified nucleic acid; or
   (vi) any combination of (i), (ii), and (v).

In embodiments, the kit comprises: a labeled probe that comprises a detection oligonucleotide comprising RNA, a modified nucleic acid, or a combination thereof; and a nuclease that is capable of cleaving the template oligonucleotide. In embodiments, the kit comprises: a labeled probe that comprises a detection oligonucleotide comprising RNA, a modified nucleic acid, or a combination thereof; a nuclease that is capable of cleaving the template oligonucleotide; and an anchoring reagent. In embodiments, the kit comprises: a labeled probe that comprises a detection oligonucleotide comprising RNA, a modified nucleic acid, or a combination thereof; a nuclease that is capable of cleaving the template oligonucleotide; an anchoring reagent; and a surface. In embodiments, the kit comprises: a labeled probe that comprises a detection oligonucleotide comprising RNA, a modified nucleic acid, or a combination thereof; a nuclease that is capable of cleaving the template oligonucleotide; and a surface comprising an anchoring reagent. In embodiments, the anchoring reagent comprises an anchoring oligonucleotide, wherein the anchoring oligonucleotide comprises a modified nucleic acid. In embodiments, the detection oligonucleotide comprises a sequence of any one of SEQ ID NOs:7-10. In embodiments, the nuclease comprises one or more restriction enzymes as shown in Table 1. In embodiments, the nuclease comprises DdeI, AluI, HpaII, ApoI, DpnI, DpnII, ScrFI, MboI, MluCI, AseI, TaqI, or combination thereof. In embodiments, the template oligonucleotide comprises a sequence of any one of SEQ ID NOs:5, 6, or 16. In embodiments, the anchoring reagent comprises an anchoring oligonucleotide comprising a sequence of any one of SEQ ID NOs:11, 17, or 20-37.

In embodiments, the kit comprises: a labeled probe that comprises a detection oligonucleotide comprising RNA, a modified nucleic acid, or a combination thereof; and an anchoring reagent. In embodiments, the kit comprises: a labeled probe that comprises a detection oligonucleotide comprising RNA, a modified nucleic acid, or a combination thereof; an anchoring reagent; and a surface. In embodiments, the kit comprises: a labeled probe that comprises a detection oligonucleotide comprising RNA, a modified nucleic acid, or a combination thereof; and a surface comprising an anchoring reagent. In embodiments, the anchoring reagent comprises an anchoring oligonucleotide, wherein the anchoring oligonucleotide comprises a modified nucleic acid. In embodiments, the detection oligonucleotide comprises a sequence of any one of SEQ ID NOs:7-10. In embodiments, the anchoring reagent comprises an anchoring oligonucleotide comprising a sequence of any one of SEQ ID NOs:11, 17, or 20-37.

In embodiments, the kit comprises: a nuclease that is capable of cleaving the template oligonucleotide; and an anchoring reagent. In embodiments, the kit comprises: a nuclease that is capable of cleaving the template oligonucleotide; an anchoring reagent; and a surface. In embodiments, the kit comprises: a nuclease that is capable of cleaving the template oligonucleotide; and a surface comprising an anchoring reagent. In embodiments, the anchoring reagent comprises an anchoring oligonucleotide, wherein the anchoring oligonucleotide comprises a modified nucleic acid. In embodiments, the nuclease comprises one or more restriction enzymes as shown in Table 1. In embodiments, the nuclease comprises DdeI, AluI, HpaII, ApoI, DpnI, DpnII, ScrFI, MboI, MluCI, AseI, TaqI, or combination thereof. In embodiments, the template oligonucleotide comprises a sequence of any one of SEQ ID NOs:5, 6, or 16. In embodiments, the anchoring reagent comprises an anchoring oligonucleotide comprising a sequence of any one of SEQ ID NOs:11, 17, or 20-37.

In embodiments, the kit comprises: a labeled probe that comprises a detection oligonucleotide comprising RNA, a modified nucleic acid, or a combination thereof; a nuclease that is capable of cleaving the template oligonucleotide; and an anchoring reagent. In embodiments, the kit comprises: a labeled probe that comprises a detection oligonucleotide comprising RNA, a modified nucleic acid, or a combination thereof; a nuclease that is capable of cleaving the template oligonucleotide; an anchoring reagent; and a surface. In embodiments, the kit comprises: a labeled probe that comprises a detection oligonucleotide comprising RNA, a modified nucleic acid, or a combination thereof; a nuclease that is capable of cleaving the template oligonucleotide; and a surface comprising an anchoring reagent. In embodiments, the anchoring reagent comprises an anchoring oligonucleotide, wherein the anchoring oligonucleotide comprises a modified nucleic acid. In embodiments, the anchoring reagent comprises an anchoring oligonucleotide, wherein the anchoring oligonucleotide comprises a modified nucleic acid. In embodiments, the detection oligonucleotide comprises a sequence of any one of SEQ ID NOs:7-10. In embodiments, the nuclease one or more restriction enzymes as shown in Table 1. In embodiments, the nuclease comprises DdeI, AluI, HpaII, ApoI, DpnI, DpnII, ScrFI, MboI, MluCI, AseI, TaqI, or combination thereof. In embodiments, the template oligonucleotide comprises a sequence of any one of SEQ ID NOs:5, 6, or 16. In embodiments, the anchoring reagent comprises an anchoring oligonucleotide comprising a sequence of any one of SEQ ID NOs:11, 17, or 20-37.

### Labeled Probe

In embodiments, the kit comprises a labeled probe that comprises a detection oligonucleotide and a detectable label. Detectable oligonucleotides and detectable labels are further described herein. In embodiments, the detectable label is capable of being detected by light scattering, optical absorbance, fluorescence, chemiluminescence, ECL, bioluminescence, phosphorescence, radioactivity, magnetic field, or combination thereof. In embodiments, the kit is for conducting a multiplexed method that is capable of detecting at least two analytes, as described herein, and the detectable label for each analyte comprises a distinct detectable signal. In embodiments, the detectable label comprises a fluorescent label, and a distinct fluorescence signature (e.g., fluorescent wavelength and/or intensity) is associated with each unique analyte, thereby allowing the analytes to be distinguished from each other. In embodiments, the detectable label is an ECL label. In embodiments, the labeled probe comprises about 1 to 10, or about 2 to 5, or about 3 to 4 ECL labels. In embodiments, the labeled probe comprises three ECL labels. ECL labels are further described herein.

In embodiments, the detection oligonucleotide of the labeled probe comprises RNA, a modified nucleic acid, or a combination thereof, as described herein. In embodiments, the detection oligonucleotide comprises RNA. In embodiments, the detection oligonucleotide comprises a modified nucleic acid. In embodiments, the detection oligonucleotide comprises a combination of RNA and a modified nucleic acid. In embodiments, the modified nucleic acid is a modified RNA nucleic acid. In embodiments, the modified nucleic acid is a modified DNA nucleic acid.

In embodiments, the modified nucleic acid comprises a PNA, an LNA, a BNA, a nucleoside comprising a 2′ modification, or a combination thereof. In embodiments, the modified nucleic acid comprises a PNA, an LNA, a BNA, a nucleoside comprising a 2′ modification, or a combination thereof. In embodiments, the 2′ modified nucleoside comprises a 2′-OMe, 2′-MOE, 2′-F, 2′-OH, or a combination thereof. In embodiments, the modified nucleic acid comprises a backbone modification, e.g., in the phosphate backbone of one or more nucleotides. In embodiments, the backbone modifications comprises a phosphorothioate, boranophosphate, methylphosphonate, phosphoramidate (e.g., morpholino phosphoramidate and mesyl phosphoramidate), phosphoramidite, 3′-O-phosphopropylamino, or combination thereof. Modified nucleic acids, e.g., PNAs, LNAs, BNAs, and nucleosides comprising a 2′ modification are further described herein.

In embodiments, the detection oligonucleotide comprises a single modified nucleic acid, e.g., a PNA monomer, LNA monomer, BNA monomer, or a single nucleoside comprising a 2′ modification. In embodiments, the detection oligonucleotide comprises more than one modified nucleic acids, e.g., more than one PNA, LNA, BNA monomers and/or nucleosides comprising a 2′ modification.

In embodiments, the detection oligonucleotide consists of modified nucleic acids, wherein each nucleotide of the detection oligonucleotide comprises a modified nucleic acid as described herein. In embodiments, each modified nucleic acid of the detection oligonucleotide comprises a PNA, an LNA, a BNA, a nucleoside comprising a 2′-modification, or combination thereof.

In embodiments, the detection oligonucleotide is about 3 to about 30 nucleotides in length, or about 4 to about 25 nucleotides in length, or about 4 to about 20 nucleotides in length, or about 5 to about 18 nucleotides in length, or about 6 to about 15 nucleotides in length, or about 8 to about 12 nucleotides in length. In embodiments, the detection oligonucleotide is about 4, about 5, about 6, about 7, about 8, about 9, about 10, about 11, about 12, about 13, about 14, about 15, about 16, about 17, about 18, about 19, or about 20 nucleotides in length. In embodiments, the detection oligonucleotide comprises or consists of a sequence as shown in Table 4 herein.

In embodiments, the detection oligonucleotide is linked to the detectable label via a conjugation linkage. Conjugation of detectable labels to oligonucleotides are known to one of ordinary skill in the art. In embodiments, the detection oligonucleotide comprises a 3′ amino modifier, an internal amino modifier, an internal spacer, or combination thereof. In embodiments, the detectable label is linked to the detection oligonucleotide via the 3′ amino modifier, internal amino modifier, and/or internal spacer. In embodiments, the detection oligonucleotide comprises a sequence as shown in Table 5. The lowercase “m” and “+” notations are as defined herein. As used herein, “iAmMC6T” refers to an amino-modified C6 dT linker; “iSp18” refers to an 18-atom hexa-ethyleneglycol spacer; and “3AmMO” refers to a 3′ amino modifier. See, e.g., “Attachment Chemistry/Linkers Modifications,” idtdna.com/site/Catalog/Modifications/Category/2.

TABLE 5
Further exemplary detection oligonucleotide
sequences

| | SEQ |
|---|---|
| | ID |
| Sequence | NO |
| mG+A+G+T+C+CmGmUmCmU/iAmMC6T/iSp18/iAmMC6T/ | 12 |
| iSp18/3AmMO/ | |
| C+A+G+T+G+AA+TGC/iAmMC6T//iSp18/iAmMC6T// | 13 |
| iSp18//3AmMO/ | |
| G+A+G+T+C+C+GTCT/iAmMC6T/iSp18/iAmMC6T/ | 14 |
| iSp18/3AmMO/ | |
| G+A+G+T+C+CGTCT/iAmMC6T/iSp18/iAmMC6T/ | 15 |
| iSp18/3AmMO/ | |

In embodiments, the invention provides an oligonucleotide comprising a sequence of any one of SEQ ID NOs:12-15. In embodiments, the invention provides an oligonucleotide consisting of a sequence of any one of SEQ ID NOs:12-15.

### Polymerase

In embodiments, the kit comprises: a labeled probe that comprises a detection oligonucleotide as described herein; and a polymerase. Polymerases are further described herein. In embodiments, the polymerase is capable of extending the nucleic acid primer on the detection reagent. In embodiments, the polymerase is capable of performing PCR, NEAR, or an isothermal amplification method such as SDA, HDA, or RCA. In embodiments, the polymerase is capable of performing MDA. In embodiments, the polymerase comprises strand-displacement activity (i.e., is an SD polymerase). In embodiments, the SD polymerase comprises an amino acid sequence of at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a DNA polymerase from a bacteriophage, e.g., Phi29, Nf, Karezi, or BeachBum. In embodiments, the SD polymerase comprises an amino acid sequence of at least 80% or at least 90% sequence identity to a DNA polymerase from a bacteriophage, wherein the bacteriophage is Phi29, Nf, Karezi, or BeachBum. In embodiments, the SD polymerase is a DNA polymerase a bacteriophage, wherein the bacteriophage is Phi29, Nf, Karezi, or BeachBum. In embodiments, activity of the polymerase is substantially uninhibited by the detection oligonucleotide described herein.

### Template Oligonucleotide

In embodiments, the kit comprises a template oligonucleotide, wherein the template oligonucleotide is capable of hybridizing to the nucleic acid primer and that comprises a same sequence as the detection oligonucleotide, which enables generation of an extended oligonucleotide comprising a sequence that is complementary to the detection oligonucleotide. Template oligonucleotides are further described herein. In embodiments, the template oligonucleotide is a template for a nucleic acid amplification by a polymerase as described herein. In embodiments, the template oligonucleotide is about 40 to about 100 nucleotides in length, or about 50 to about 78 nucleotides in length, or about 53 to about 76 nucleotides in length, or about 50 to about 70 nucleotides in length, or about 53 to about 61 nucleotides in length, or about 54 to about 61 nucleotides in length. In embodiments, the template oligonucleotide is about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, or about 76 nucleotides in length.

In embodiments, the template oligonucleotide comprises one or more connector oligonucleotides, wherein the one or more connector oligonucleotides are capable of being ligated to form a circular template. In embodiments, the 5′ and 3′ ends of the template oligonucleotide are capable of hybridizing to first and second regions of the nucleic acid primer on the detection reagent. In embodiments, the template oligonucleotide is a circular oligonucleotide. In embodiments, the template oligonucleotide is a linear oligonucleotide, wherein the 5′ and 3′ ends of the linear oligonucleotide are capable of being ligated to form a circular oligonucleotide. In embodiments, the template oligonucleotide comprises 5′-GTTCTGTC-3′ at its 5′ end and 5′-GTGTCTA-3′ at its 3′ end. In embodiments, the template oligonucleotide comprises or consists of a sequence shown in Table 3 herein. In embodiments, the template oligonucleotide is 5′-phosphorylated. In embodiments, the 5′-phosphorylation of the template oligonucleotide enables ligation of its 5′ and 3′-ends as described herein. In embodiments, the template oligonucleotide comprises the sequence

(SEQ ID NO: 16)
/5Phos/GTTCTGTCATATTTCAGTGAATGCGAGTCCGTCTAAGAGAG
TAGTACAGCAAGAGTGTCTA

In embodiments, the template oligonucleotide comprises a sequence of SEQ ID NO:5 or 6. In embodiments, the template oligonucleotide consists of a sequence of SEQ ID NO:5 or 6.

### Nuclease

In embodiments, the kit comprises a nuclease that is capable of cleaving the template oligonucleotide. Nucleases are further described herein. In embodiments, the nuclease specifically cleaves the template oligonucleotide. In embodiments, the nuclease does not cleave the nucleic acid primer or the extended oligonucleotide. In embodiments, the nuclease specifically cleaves double-stranded oligonucleotides, e.g., double-stranded DNA, double-stranded RNA, or a double-stranded DNA/RNA hybrid. In embodiments, the double-stranded DNA, double-stranded RNA, or double-stranded DNA/RNA hybrid comprises the template oligonucleotide and the nucleic acid primer hybridized thereto.

In embodiments, the nuclease cleaves a double-stranded portion of the template oligonucleotide that is hybridized to the nucleic acid primer. In embodiments, the template oligonucleotide and the nucleic acid primer each comprises single-stranded DNA. In embodiments, the nuclease is a restriction endonuclease. Exemplary restriction endonucleases are provided herein. In embodiments, the nuclease comprises one or more restriction enzymes as shown in Table 1. In embodiments, the nuclease is DdeI, AluI, HpaII, ApoI, DpnI, DpnII, ScrFI, MboI, MluCI, AseI, TaqI, TspRI, or a combination thereof.

In embodiments, the nuclease cleaves a double-stranded portion of the template oligonucleotide that forms a DNA/RNA hybrid with the nucleic acid primer. In embodiments, the template oligonucleotide comprises single-stranded DNA, and the nucleic acid primer comprises single-stranded RNA. In embodiments, the template oligonucleotide comprises single-stranded RNA, and the nucleic acid primer comprises single-stranded DNA. Exemplary nucleases that are capable of cleaving DNA/RNA hybrids are provided herein. In embodiments, the nuclease is RNase H2.

In embodiments, the template oligonucleotide comprises a DNA damage indicator, and the nuclease comprises an excision enzyme that specifically binds to the DNA damage indicator and cleaves the template oligonucleotide. In embodiments, the DNA damage indicator is not present in the nucleic acid primer or the extended oligonucleotide, and the nuclease does not bind to or cleave the nucleic acid primer or the extended oligonucleotide. DNA damage indicators and their corresponding excision enzymes are further described herein. In embodiments, the DNA damage indicator comprises an uracil base, and the nuclease comprises UNG. In embodiments, the kit further comprises an abasic site endonuclease, which further assists in cleavage of the template oligonucleotide by UNG. In embodiments, the abasic site endonuclease comprises UDG, APE1, Endonuclease IV, or a combination thereof. In embodiments, the DNA damage indicator comprises deoxyinosine, and the nuclease comprises Endonuclease V. In embodiments, the DNA damage indicator comprises a damaged purine, and the nuclease comprises an enzyme that repairs the damaged purine. In embodiments, the damaged purine comprises 8oxoG, and the template-cleaving enzyme comprises Fpg.

### Anchoring Reagent

In embodiments, the kit comprises an anchoring reagent. In embodiments, the anchoring reagent is lyophilized. In embodiments, the anchoring reagent is provided in solution. Anchoring reagents are further described herein. In embodiments, the template oligonucleotide of the kit comprises a region comprising a same sequence as an anchoring oligonucleotide, which enables generation of an extended oligonucleotide comprising a sequence that is complementary to the anchoring oligonucleotide. In embodiments, the template oligonucleotide of the kit comprises (i) a first region comprising a same sequence as a detection oligonucleotide as described herein and (ii) a second region comprising a same sequence as an anchoring oligonucleotide, which enables generation of an extended oligonucleotide comprising (I) a first sequence that is complementary to the detection oligonucleotide and (II) a second sequence that is complementary to the anchoring oligonucleotide.

In embodiments, the anchoring reagent comprises an oligonucleotide, aptamer, aptamer ligand, antibody, antigen, ligand, receptor, hapten, epitope, or mimotope. In embodiments, the anchoring reagent comprises an anchoring oligonucleotide. In embodiments, the anchoring oligonucleotide comprises a single stranded oligonucleotide. In embodiments, the anchoring oligonucleotide comprises a double stranded oligonucleotide.

In embodiments, the anchoring oligonucleotide comprises a modified nucleic acid. Modified nucleic acids are further described herein. In embodiments, the modified nucleic acid comprises a modified base, modified sugar, and/or modified backbone. In embodiments, the modified nucleic acid comprises a PNA, an LNA, a BNA, a nucleoside comprising a 2′ modification, or a combination thereof. In embodiments, the nucleoside comprising a 2′ modification comprises a 2′-OMe, a 2′-MOE, a 2′-F, a 2′-OH, or combination thereof. In embodiments, the modified nucleic acid comprises a backbone modification, e.g., in the phosphate backbone of one or more nucleotides. In embodiments, the backbone modifications comprises a phosphorothioate, boranophosphate, methylphosphonate, phosphoramidate (e.g., morpholino phosphoramidate and mesyl phosphoramidate), phosphoramidite, 3′-O-phosphopropylamino, or combination thereof. Modified nucleic acids, e.g., PNAs, LNAs, BNAs, and nucleosides comprising a 2′ modification are further described herein.

In embodiments, the anchoring reagent consists of modified nucleic acids, wherein each nucleotide of the anchoring oligonucleotide comprises a modified nucleic acid as described herein. In embodiments, each modified nucleic acid of the anchoring oligonucleotide comprises a PNA, an LNA, a BNA, a nucleoside comprising a 2′-modification, or combination thereof.

In embodiments, the anchoring oligonucleotide is about 3 to about 30 nucleotides in length, or about 4 to about 25 nucleotides in length, or about 4 to about 20 nucleotides in length, or about 5 to about 18 nucleotides in length, or about 6 to about 15 nucleotides in length, or about 8 to about 12 nucleotides in length. In embodiments, the anchoring oligonucleotide is about 4, about 5, about 6, about 7, about 8, about 9, about 10, about 11, about 12, about 13, about 14, about 15, about 16, about 17, about 18, about 19, or about 20 nucleotides in length. In embodiments, the anchoring oligonucleotide comprises or consists of the sequence mU+AmGmUmA+C+AmGmC (SEQ ID NO:11). In embodiments, the anchoring oligonucleotide comprises a biotin at a 3′-end, e.g., for immobilization of the anchoring reagent to a surface as described herein. In embodiments, the anchoring oligonucleotide comprises the sequence mU+AmGmUmA+C+AmGmC/3Bio/(SEQ ID NO:17). In embodiments, the anchoring oligonucleotide comprises a thiol at a 3′-end, e.g., for immobilization of the anchoring reagent to a surface as described herein. In embodiments, the anchoring oligonucleotide comprises a sequence of any one of SEQ ID NOs:20-37.

### Capture Reagent

In embodiments, the kit comprises a capture reagent. In embodiments, the capture reagent is lyophilized. In embodiments, the capture reagent is provided in solution. Capture reagents are further described herein. In embodiments, the capture reagent comprises a protein or polypeptide, antibody or antigen-binding fragment thereof, antigen, ligand, receptor, oligonucleotide, hapten, epitope, mimotope, or aptamer. In embodiments, the capture reagent comprises an antibody or a variant thereof, including an antigen/epitope-binding portion thereof, an antibody fragment or derivative, an antibody analogue, an engineered antibody, or a substance that binds to antigens in a similar manner to antibodies. In embodiments, the capture reagent at least one heavy or light chain complementarity determining region (CDR) of an antibody. In embodiments, the capture reagent comprises at least two CDRs from one or more antibodies. In embodiments, the capture reagent comprises an antibody or antigen-binding fragment thereof. In embodiments, the capture reagent comprises an antigen-binding domain that specifically binds to an epitope of the analyte. In embodiments, the capture reagent comprises an oligonucleotide. In embodiments, the analyte comprises an oligonucleotide, and the capture reagent comprises an oligonucleotide that is complementary to the analyte.

### Surface

In embodiments, the kit comprises a surface, and each of the capture reagent and the anchoring reagent is capable of being immobilized to the surface. In embodiments, the kit comprises a surface, and each of the capture reagent and the anchoring reagent is provided on the surface. In embodiments, the kit comprises a surface, wherein the anchoring reagent is immobilized on the surface, and the capture reagent is not provided on the surface and is capable of being immobilized on the surface. In embodiments, the kit comprises a surface, wherein the capture reagent is immobilized on the surface, and the anchoring reagent is not provided on the surface and is capable of being immobilized on the surface. Immobilization of capture and/or anchoring reagents onto surfaces is further described herein.

In embodiments, the capture reagent is immobilized or capable of being immobilized on the surface via a covalent linkage between the capture reagent and the surface, e.g., a reaction between a thiol group of the capture reagent and the surface. In embodiments, the capture reagent is linked to a first binding partner, which is capable of binding to a second binding partner that is immobilized on the surface. In embodiments, the first and second binding partners comprise complementary oligonucleotides, a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the first and second binding partners comprise cross-reactive moieties, e.g., thiol and maleimide or iodoacetamide; aldehyde and hydrazide; or azide and alkyne or cycloalkyne. In embodiments, the first binding partner comprises biotin, and the second binding partner comprises avidin, streptavidin, an anti-biotin antibody, or a combination thereof. In embodiments, the first and second binding partners bind to each other via a bridging agent, which binds both the first and second binding partners. In embodiments, the bridging agent comprises at least two binding sites, wherein each of the first and second binding partners bind to a distinct binding site. In embodiments, the bridging agent comprises streptavidin or avidin, and the first and second binding partners are each biotin.

In embodiments, the anchoring reagent is immobilized or capable of being immobilized on the surface via a covalent linkage between the anchoring reagent and the surface, e.g., a reaction between a thiol group of the anchoring reagent and the surface. In embodiments, the anchoring reagent is linked to a first binding partner, which is capable of binding to a second binding partner that is immobilized on the surface. In embodiments, the first and second binding partners comprise complementary oligonucleotides, a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the first and second binding partners comprise cross-reactive moieties, e.g., thiol and maleimide or iodoacetamide; aldehyde and hydrazide; or azide and alkyne or cycloalkyne. In embodiments, the first binding partner comprises biotin, and the second binding partner comprises avidin, streptavidin, an anti-biotin antibody, or a combination thereof. In embodiments, the first and second binding partners bind to each other via a bridging agent, which binds both the first and second binding partners. In embodiments, the bridging agent comprises at least two binding sites, wherein each of the first and second binding partners bind to a distinct binding site. In embodiments, the bridging agent comprises streptavidin or avidin, and the first and second binding partners are each biotin.

In embodiments, the anchoring reagent comprises an anchoring oligonucleotide and a first binding partner, wherein the first binding partner is linked to a nucleotide of the anchoring oligonucleotide. In embodiments, the first binding partner is linked to an internal nucleotide of the anchoring oligonucleotide. In embodiments, the first binding partner is positioned at a 5′-end of the anchoring reagent. In embodiments, the first binding partner is positioned at a 3′-end of the anchoring reagent. In embodiments, the first binding partner is linked to a 5′- or 3-terminal nucleotide of the anchoring oligonucleotide. In embodiments, the anchoring reagent comprises a spacer positioned between the first binding partner and the anchoring oligonucleotide. In embodiments, the spacer comprises a PEG comprising about 1 to about 50, or about 2 to about 40, or about 3 to about 30, or about 4 to about 20, or about 5 to about 10, or about 1 to about 15, or about 2 to about 10, or about 3 to about 8, or about 4 to about 7, or about 5 to about 6 ethylene glycol units. In embodiments, the spacer comprises a PEG comprising about 1, 2, 3, 4, 5, 6, 7, 8, 9, 10, 11, 12, 13, 14, 15, 16, 17, 18, 19, or 20 ethylene glycol units. In embodiments, the first binding partner is positioned at a 3′-end of the anchoring reagent, and the anchoring reagent comprises a PEG spacer comprising about 1 to about 15, or about 2 to about 10, or about 3 to about 8 ethylene glycol units. In embodiments, the anchoring reagent comprises a 3′-terminal nucleotide that is linked to a first end of a PEG spacer and a first binding partner that is linked to a second end of a PEG spacer, wherein the PEG spacer comprises about 1 to about 15, or about 2 to about 10, or about 3 to about 8 ethylene glycol units.

In embodiments, the first binding partner of the capture reagent and the first binding partner of the anchoring reagent are substantially non-cross reactive, i.e., the first binding partners of the capture reagent and the anchoring reagent bind to different second binding partners on the surface. In embodiments, the first binding partner of the capture reagent and the first binding partner of the anchoring reagent are capable of binding to the same second binding partner on the surface.

Surfaces are further described herein. In embodiments, the surface comprises a particle. In some embodiments, the particle comprises a microsphere. In embodiments, the particle comprises a paramagnetic bead. In embodiments, the surface comprises a cartridge. In embodiments, the surface comprises a well of multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface comprises a plurality of distinct binding domains, and the capture reagent and anchoring reagent are immobilized or capable of being immobilized on two distinct binding domains on the surface. In embodiments, the surface comprises a plurality of distinct binding domains, and the capture reagent and anchoring reagent are immobilized or capable of being immobilized on the same binding domain on the surface. In embodiments, the surface comprises a particle, wherein the capture reagent and the anchoring reagent are immobilized or capable of being immobilized on the same particle. In embodiments, the capture reagent is within about 1 nm to about 500 nm, about 5 nm to about 250 nm, about 10 nm to about 200 nm, or about 15 nm to about 150 nm of the anchoring reagent on the surface. In embodiments, the capture reagent is less than 1 m from the anchoring reagent on the surface. In embodiments, the capture reagent is less than 500 nm from the anchoring reagent on the surface. In embodiments, the capture reagent is less than 200 nm from the anchoring reagent on the surface.

In embodiments, the surface comprises an electrode. In embodiments, the electrode comprises a carbon ink electrode. In embodiments, the surface comprises a particle, and the kit further comprises an electrode for collecting the particle. In embodiments, the kit further comprises a reagent for immobilizing a capture and/or anchoring reagent to the surface.

### Detection Reagent

In embodiments, the kit comprises a detection reagent, wherein the detection reagent comprises a protein or a polypeptide. In embodiments, the detection reagent is lyophilized. In embodiments, the detection reagent is provided in solution. Detection reagents are further described herein. In embodiments, the detection reagent comprises an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent comprises an antibody or antigen-binding fragment thereof. In embodiments, the detection reagent comprises an antigen-binding domain that specifically binds to an epitope of the analyte. In embodiments, the detection reagent comprises a protein or polypeptide antigen. In embodiments, the detection reagent comprises a protein or polypeptide ligand or receptor.

In embodiments, the detection reagent comprises a nucleic acid primer or is capable of being linked to a nucleic acid primer. Nucleic acid primers and conjugation thereof with proteins or polypeptides, e.g., antibodies or antigen-binding fragments thereof, are further described herein. In embodiments, the nucleic acid primer comprises a conjugation moiety for conjugation to the detection reagent. In embodiments, the conjugation moiety is at a 5′-end or a 3′-end of the nucleic acid primer. In embodiments, the conjugation moiety comprises a thiol. In embodiments, the nucleic acid primer comprises a 5′-thiol.

In embodiments, the nucleic acid primer is about 10 to about 30 nucleotides in length, or about 12 to about 28 nucleotides in length, or about 13 to about 26 nucleotides in length, or about 14 to about 24 nucleotides in length, or about 11 to about 22 nucleotides in length, or about 12 to about 21 nucleotides in length, or about 13 to about 20 nucleotides in length, or about 13 to about 18 nucleotides in length, or about 14 to about 19 nucleotides in length. In embodiments, the nucleic acid primer is about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides in length. In embodiments, the nucleic acid primer is about 14 nucleotides in length or about 15 nucleotides in length. In embodiments, the nucleic acid primer comprises or consists of a sequence as described in Table 2 herein. In embodiments, the nucleic acid primer comprises the sequence/5ThioMC6-D/GACAGAACTAGACAC (SEQ ID NO:18), wherein “5ThioMC6-D” refers to a 5′ thiol modifier C6 S—S modification. In embodiments, the 5′ thiol modifier enables conjugation of the nucleic acid primer to the detection reagent as described herein.

### ADDITIONAL EMBODIMENTS

In embodiments, the kit further comprises a calibration reagent, a blocking reagent, a diluent, a stabilizing agent, a buffer, a ligase, a reagent for conjugating the nucleic acid primer to the detection reagent, a co-reactant for the detectable label, a detergent, a salt, a preservative, or a combination thereof.

In embodiments, the kit comprises a calibration reagent. In embodiments, the calibration reagent comprises a known quantity of the analyte. In embodiments, the kit comprises multiple calibration reagents comprising a range of concentrations of the analyte. In embodiments, the multiple calibration reagents comprise concentrations of the analyte near the upper and lower limits of quantitation for the method. In embodiments, the multiple calibration reagents span the entire dynamic range of the method. In embodiments, the calibration reagent is a positive control reagent. In embodiments, the calibration reagent is a negative control reagent. In embodiments, the positive or negative control reagent is used to provide a basis of comparison for the sample to be tested with the methods of the invention. In embodiments, the calibration reagent is lyophilized. In embodiments, the calibration reagent is provided in solution.

In embodiments, the kit comprises a blocking reagent. In embodiments, the blocking reagent decreases non-specific binding by components other than tau to the capture and detection reagents described herein. Exemplary blocking agents include, but are not limited to, mBSA, sheared poly(A), polyBSA-I, mIgG, Tween, polyBSA-II, yeast RNA, mBSA+poly(a), and/or polyBSA+poly(A). In embodiments, the kit further comprises a diluent for one or more components of the kit. In embodiments, a kit comprising the components above includes stock concentrations of the components that are 5×, 10×, 20×, 30×, 40×, 50×, 60×, 70×, 80×, 90×, 100×, 125×, 150× or higher fold concentrations of a working concentration for the methods provided herein. In embodiments, the kit further comprises a stabilizing agent, e.g., for storage of one or more components of the kit.

In embodiments, the kit comprises a buffer, e.g., an assay buffer, a reconstitution buffer, a storage buffer, a read buffer, or a combination thereof. In embodiments, the kit further comprises a co-reactant, e.g., for performing an electrochemiluminescence measurement. Exemplary co-reactants are described, e.g., in WO 2020/142313.

In embodiments, the kit comprises a ligase. In embodiments, the ligase is capable of ligating a linear template oligonucleotide provided herein to form a circular template oligonucleotide as described herein. In embodiments, the ligase is T4 DNA ligase, T7 DNA ligase, Taq DNA ligase, ELECTROLIGASE®, SPLINTR® ligase, or combination thereof.

In embodiments, the kit comprises a reagent for conjugating the nucleic acid primer to the detection reagent. Exemplary reagents are further described in, e.g., WO 2021/092004 and Wong, S. S. and Jameson, D. M., Chemistry of Protein and Nucleic Acid Cross-Linking and Conjugation, 2^{nd} Ed., CRC Press (2011).

In embodiments, the kit further comprises an assay consumable, e.g., assay modules, vials, tubes, liquid handling and transfer devices such as pipette tips, covers and seals, racks, labels, and the like. In embodiments, the kit further comprises an electrode, e.g., for performing an ECL measurement. In embodiments, the electrode is applied to the surface provided herein. In embodiments, the kit further comprises an assay instrument and/or instructions for carrying out the methods described herein.

It will be understood by one of ordinary skill in the art that components of the kits described herein, which may be provided in one or more vials, containers, or compartments, are not necessarily included in the same container, e.g., same box, and/or at the same time. In embodiments, the components of the kits described herein are provided in one or more separate containers or compartments either simultaneously or sequentially. It will be further understood by one of ordinary skill in the art that a user may obtain (e.g., purchase or possess) the components of the kit (e.g., the signal amplification reagent described herein) separately, e.g., in one or more separate containers or compartments, but nonetheless the components are considered part of a “kit” when used in combination, e.g., as described in embodiments herein. In some embodiments, a kit comprises multiple containers, vials, or compartments supplied together in a single package or container. In embodiments, the components of the kits described herein are provided separately, e.g., according to the components' optimal shipping or storage temperatures.

### Compositions

In embodiments, the invention provides a composition for labeling a surface, comprising: a labeled probe that comprises (1) a detectable label; and (2) detection oligonucleotide that is capable of binding to an extended oligonucleotide that is bound to the surface, wherein the extended oligonucleotide is formed by extension of a nucleic acid primer by a polymerase based on a template oligonucleotide, and wherein: (i) the detection oligonucleotide comprises RNA, a modified nucleic acid, or a combination thereof; (ii) the composition further comprises a nuclease that is capable of cleaving the template oligonucleotide; (iii) combination of (i) and (ii); (iv) one or both of (i) and (ii), and further wherein the extended oligonucleotide is bound to the surface via an anchoring reagent; (v) the surface comprises an anchoring reagent that comprises an anchoring oligonucleotide, wherein the anchoring oligonucleotide comprises a modified nucleic acid; or (vi) any combination of (i), (ii), and (v). In embodiments, the composition comprises the detection oligonucleotide comprising RNA, a modified nucleic acid, or a combination thereof; and the nuclease that is capable of cleaving the template oligonucleotide. In embodiments, the composition comprises the detection oligonucleotide comprising RNA, a modified nucleic acid, or a combination thereof; and the anchoring reagent that comprises an anchoring oligonucleotide, wherein the anchoring oligonucleotide comprises a modified nucleic acid. In embodiments, the composition comprises the nuclease that is capable of cleaving the template oligonucleotide; and the anchoring reagent that comprises an anchoring oligonucleotide, wherein the anchoring oligonucleotide comprises a modified nucleic acid. In embodiments, the composition comprises the detection oligonucleotide comprising RNA, a modified nucleic acid, or a combination thereof; the nuclease that is capable of cleaving the template oligonucleotide; and the anchoring reagent that comprises an anchoring oligonucleotide, wherein the anchoring oligonucleotide comprises a modified nucleic acid. In embodiments, the template oligonucleotide is a circular oligonucleotide. In embodiments, the extension of the nucleic acid primer is by rolling circle amplification (RCA).

In embodiments, the invention provides a composition for labeling a surface, comprising: (a) a nucleic acid primer that is immobilized directly or indirectly on a surface; (b) a template oligonucleotide comprising (1) a first region that is complementary to the nucleic acid primer; and (2) a second region that comprises a same sequence as a detection oligonucleotide; (c) a polymerase; and (d) a labeled probe comprising (1) a detectable label; and (2) detection oligonucleotide that is capable of binding to an extended oligonucleotide that is bound to the surface, wherein an extended oligonucleotide is formed by extension of the nucleic acid primer by the polymerase based on the template oligonucleotide, and wherein: (i) the detection oligonucleotide comprises RNA, a modified nucleic acid, or a combination thereof; (ii) the composition further comprises a nuclease that is capable of cleaving the template oligonucleotide; (iii) combination of (i) and (ii); (iv) one or both of (i) and (ii), and further wherein the extended oligonucleotide is bound to the surface via an anchoring reagent; (v) the surface comprises an anchoring reagent that comprises an anchoring oligonucleotide, wherein the anchoring oligonucleotide comprises a modified nucleic acid; or (vi) any combination of (i), (ii), and (v). In embodiments, the composition comprises the detection oligonucleotide comprising RNA, a modified nucleic acid, or a combination thereof; and the nuclease that is capable of cleaving the template oligonucleotide. In embodiments, the composition comprises the detection oligonucleotide comprising RNA, a modified nucleic acid, or a combination thereof; and the anchoring reagent that comprises an anchoring oligonucleotide, wherein the anchoring oligonucleotide comprises a modified nucleic acid. In embodiments, the composition comprises the nuclease that is capable of cleaving the template oligonucleotide; and the anchoring reagent that comprises an anchoring oligonucleotide, wherein the anchoring oligonucleotide comprises a modified nucleic acid. In embodiments, the composition comprises the detection oligonucleotide comprising RNA, a modified nucleic acid, or a combination thereof; the nuclease that is capable of cleaving the template oligonucleotide; and the anchoring reagent that comprises an anchoring oligonucleotide, wherein the anchoring oligonucleotide comprises a modified nucleic acid. In embodiments, the template oligonucleotide is a circular oligonucleotide. In embodiments, the extension of the nucleic acid primer is by rolling circle amplification (RCA).

In embodiments, the composition comprises the detection oligonucleotide comprising RNA, a modified nucleic acid, or a combination thereof. In embodiments, the detection oligonucleotide comprises a modified nucleic acid. In embodiments, the composition is suitable for detecting an analyte, e.g., that is present on the surface. In embodiments, the composition is suitable for generating a detectable signal from the detectable label. In embodiments, the detection oligonucleotide comprises a sequence of any one of SEQ ID NOs:7-10.

Labeled probes, including detectable labels and detection oligonucleotides, are further described herein. In embodiments, the detectable label is an ECL label. In embodiments, the detection oligonucleotide comprises a modified nucleic acid. In embodiments, the modified nucleic acid comprises a PNA, an LNA, a BNA, a nucleoside comprising a 2′ modification, or a combination thereof. In embodiments, the nucleoside comprising the 2′ modification comprises a 2′-OMe, 2′-MOE, 2′-F, 2′-OH, or a combination thereof. In embodiments, the modified nucleic acid comprises a backbone modification, e.g., a phosphorothioate, boranophosphate, methylphosphonate, phosphoramidate (e.g., morpholino phosphoramidate and mesyl phosphoramidate), phosphoramidite, 3′-O-phosphopropylamino, or combination thereof as described herein. Modified nucleic acids are further described herein.

In embodiments, the detection oligonucleotide is about 3 to about 30 nucleotides in length, or about 4 to about 25 nucleotides in length, or about 4 to about 20 nucleotides in length, or about 5 to about 18 nucleotides in length, or about 6 to about 15 nucleotides in length, or about 8 to about 12 nucleotides in length. In embodiments, the detection oligonucleotide is about 4, about 5, about 6, about 7, about 8, about 9, about 10, about 11, about 12, about 13, about 14, about 15, about 16, about 17, about 18, about 19, or about 20 nucleotides in length. In embodiments, the detection oligonucleotide comprises or consists of a sequence as shown in Table 4 herein.

In embodiments, the composition further comprises one or more of the polymerase, the primer, the template oligonucleotide, or a combination thereof.

In embodiments, the composition comprises a polymerase, wherein the polymerase is an SD polymerase as described herein. In embodiments, the SD polymerase comprises an amino acid sequence of at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a DNA polymerase from a bacteriophage. In embodiments, the bacteriophage is Phi29, Nf, Karezi, or BeachBum. In embodiments, the SD polymerase comprises an amino acid sequence of at least 80% sequence identity to a DNA polymerase from a bacteriophage, wherein the bacteriophage is Phi29, Nf, Karezi, or BeachBum. In embodiments, the SD polymerase comprises an amino acid sequence of at least 90% sequence identity to a DNA polymerase a bacteriophage, wherein the bacteriophage is Phi29, Nf, Karezi, or BeachBum. In embodiments, the SD polymerase is a DNA polymerase a bacteriophage, wherein the bacteriophage is Phi29, Nf, Karezi, or BeachBum.

In embodiments, the composition comprises a nucleic acid primer, wherein the nucleic acid primer is linked to a detection reagent as described herein. In embodiments, the detection reagent comprises a protein or polypeptide. In embodiments, the detection reagent comprises an antibody or antigen-binding fragment thereof. In embodiments, the extended oligonucleotide is formed by extending the nucleic acid primer, e.g., by a polymerase as described herein.

In embodiments, the composition comprises a template oligonucleotide, e.g., for nucleic acid amplification as described herein. Template oligonucleotides are further described herein. In embodiments, the composition comprises the polymerase, the nucleic acid primer, and the template oligonucleotide as described herein. In embodiments, the template oligonucleotide is capable of binding to the nucleic acid primer, and the polymerase is capable of extending the nucleic acid primer from the template oligonucleotide by PCR, NEAR, and/or an isothermal amplification method such as SDA, HDA, RCA, or combination thereof, to form an extended oligonucleotide as described herein. In embodiments, the template oligonucleotide comprises or consists of a sequence as shown in Table 3 herein. In embodiments, the template oligonucleotide is a circular template oligonucleotide formed by ligating the 5′ and 3′-ends of a sequence as shown in Table 3 herein.

In embodiments, the composition comprises the nuclease that is capable of cleaving the template oligonucleotide. Nucleases are further described herein. In embodiments, the nuclease comprises one or more restriction enzymes as shown in Table 1. In some embodiments, the nuclease comprises DdeI, AluI, HpaII, ApoI, DpnI, DpnII, ScrFI, MboI, MluCI, AseI, TaqI, or combination thereof.

In embodiments, the composition comprises the extended oligonucleotide on the surface, wherein the extended oligonucleotide is formed by polymerase extension of the nucleic acid primer from the template oligonucleotide. Extended oligonucleotides are further described herein. In embodiments, the extended oligonucleotide is about 100 to about 100000 bases, or about 200 to about 75000 bases, or about 500 to about 50000 bases, or about 700 to about 20000 bases, or about 1000 to about 15000 bases, or about 2000 to about 10000 bases, or about 3000 to about 8000 bases, or about 4000 to about 7000 bases, or about 5000 to about 6000 bases in length. In embodiments, the extended oligonucleotide is about 100 to about 80000 bases, or about 200 to about 60000 bases, or about 500 to about 50000 bases, or about 4000 to about 100000 bases, or about 7500 to about 75000 bases, or about 9000 to about 40000 bases, or about 1000 to about 50000 bases, or about 2000 to about 25000 bases, about 3000 to about 13000 bases in length. In embodiments, the extended oligonucleotide is about 100 to about 8000 bases, or about 500 to about 6000 bases, or about 1000 to about 4500 bases in length.

In embodiments, the extended oligonucleotide is bound to the surface via an anchoring reagent. Anchoring reagents are further described herein. In embodiments, the anchoring reagent comprises an anchoring oligonucleotide. In embodiments, the anchoring oligonucleotide comprises a modified nucleic acid as described herein. In embodiments, the modified nucleic acid comprises a PNA, an LNA, a BNA, a nucleoside comprising a 2′ modification, or a combination thereof. In embodiments, the nucleoside comprising the 2′ modification comprises a 2′-OMe, 2′-MOE, 2′-F, 2′-OH, or a combination thereof. In embodiments, the modified nucleic acid comprises a backbone modification, e.g., a phosphorothioate, boranophosphate, methylphosphonate, phosphoramidate (e.g., morpholino phosphoramidate and mesyl phosphoramidate), phosphoramidite, 3′-O-phosphopropylamino, or combination thereof as described herein. Modified nucleic acids are further described herein.

In embodiments, the invention provides a composition comprising: a capture reagent, an analyte, a detection reagent that comprises a nucleic acid primer, a template oligonucleotide, a polymerase, and a nuclease. Components of the composition, i.e., the capture reagent, analyte, detection reagent, nucleic acid primer, template oligonucleotide, polymerase, and nuclease are further described herein. In embodiments, each of the capture reagent and the detection reagent comprises an antibody or antigen-binding fragment thereof. In embodiments, the nucleic acid primer comprises a sequence of any one of SEQ ID NOs:1-4. In embodiments, the template oligonucleotide comprises a sequence of any one of SEQ ID NOs:5, 6, or 16. In embodiments, the polymerase comprises an amino acid sequence having at least 90% sequence identity to a DNA polymerase from a bacteriophage, wherein the bacteriophage is Phi29, Nf, Karezi, or BeachBum. In embodiments, the nuclease comprises one or more restriction enzymes as shown in Table 1. In some embodiments, the nuclease comprises DdeI, AluI, HpaII, ApoI, DpnI, DpnII, ScrFI, MboI, MluCI, AseI, TaqI, or combination thereof.

In embodiments, the invention provides a composition comprising: a capture reagent, an analyte, a detection reagent that comprises an extended oligonucleotide, and an anchoring reagent that comprises an anchoring oligonucleotide. Components of the composition, i.e., the capture reagent, analyte, detection reagent, extended oligonucleotide, anchoring reagent, and anchoring oligonucleotide are further described herein. In embodiments, each of the capture reagent and the detection reagent comprises an antibody or antigen-binding fragment thereof. In embodiments, the extended oligonucleotide is extended from a template oligonucleotide of any one of SEQ ID NOs:5, 6, or 16. In embodiments, the anchoring reagent comprises an anchoring oligonucleotide comprising a sequence of any one of SEQ ID NOs:11, 17, or 20-37.

In embodiments, the invention provides a composition comprising: a capture reagent, an analyte, a detection reagent that comprises an extended oligonucleotide, and a labeled probe that comprises a detection oligonucleotide. Components of the composition, i.e., the capture reagent, analyte, detection reagent, extended oligonucleotide, labeled probe, and detection oligonucleotide are further described herein. In embodiments, each of the capture reagent and the detection reagent comprises an antibody or antigen-binding fragment thereof. In embodiments, the extended oligonucleotide is extended from a template oligonucleotide of any one of SEQ ID NOs:5, 6, or 16. In embodiments, the detection oligonucleotide comprises a sequence of any one of SEQ ID NOs:7-10.

In embodiments, the capture reagent is immobilized on the surface as described herein. In embodiments, the capture reagent and the detection reagent are bound to the analyte to form a complex on the surface. In embodiments, the detection reagent is an antibody or antigen-binding fragment thereof. In embodiments, each of the capture reagent and the detection reagent is an antibody or antigen-binding fragment thereof.

In embodiments, the composition comprises a detection reagent, wherein the detection reagent comprises a nucleic acid primer. In embodiments, the nucleic acid primer comprises a sequence as shown in Table 2. In embodiments, the composition comprises a template oligonucleotide, e.g., a circular template oligonucleotide, and the nucleic acid primer is hybridized to the template oligonucleotide. In embodiments, the template oligonucleotide comprises a sequence as shown in Table 3. In embodiments, the composition comprises a polymerase, wherein the polymerase is capable of extending the nucleic acid primer in a nucleic acid amplification reaction such as PCR, NEAR, and/or an isothermal amplification method such as SDA, HDA, RCA, or combination thereof. In embodiments, the polymerase is an SD polymerase as described herein, e.g., comprising an amino acid sequence of at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a DNA polymerase from a bacteriophage. In embodiments, the bacteriophage is Phi29, Nf, Karezi, or BeachBum. In embodiments, the composition comprises a nuclease, wherein the nuclease is capable of cleaving the template oligonucleotide, e.g., a double-stranded portion of the template oligonucleotide that is hybridized to the nucleic acid primer. In embodiments, the nuclease comprises one or more restriction enzymes as shown in Table 1. In embodiments, the nuclease is DdeI, AluI, HpaII, ApoI, DpnI, DpnII, ScrFI, MboI, MluCI, AseI, TaqI, TspRI, or a combination thereof. In embodiments, the nuclease is RNase H2. In embodiments, the nuclease is UNG, Endonuclease V, or Fpg. In embodiments, the composition further comprises an abasic site endonuclease, e.g., UDG, APE1, and/or Endonuclease IV, as described herein.

In embodiments, the composition comprises a detection reagent, wherein the detection reagent comprises an extended oligonucleotide. In embodiments, the extended oligonucleotide is formed from nucleic acid amplification of a nucleic acid primer on the detection reagent as described herein. In embodiments, the extended oligonucleotide is about 100 to about 100000 bases, or about 200 to about 75000 bases, or about 500 to about 50000 bases, or about 700 to about 20000 bases, or about 1000 to about 15000 bases, or about 2000 to about 10000 bases, or about 3000 to about 8000 bases, or about 4000 to about 7000 bases, or about 5000 to about 6000 bases in length. In embodiments, the extended oligonucleotide is about 100 to about 80000 bases, or about 200 to about 60000 bases, or about 500 to about 50000 bases, or about 4000 to about 100000 bases, or about 7500 to about 75000 bases, or about 9000 to about 40000 bases, or about 1000 to about 50000 bases, or about 2000 to about 25000 bases, about 3000 to about 13000 bases in length. In embodiments, the extended oligonucleotide is about 100 to about 8000 bases, or about 500 to about 6000 bases, or about 1000 to about 4500 bases in length.

In embodiments, the composition comprises an anchoring reagent, wherein the anchoring reagent comprises an anchoring oligonucleotide. In embodiments, the anchoring oligonucleotide comprises or consists of a sequence of any one of SEQ ID NOs:11, 17, or 20-37. In embodiments, the anchoring oligonucleotide comprises a modified nucleic acid as described herein. In embodiments, the modified nucleic acid comprises a PNA, an LNA, a BNA, a nucleoside comprising a 2′ modification, or a combination thereof. In embodiments, the nucleoside comprising the 2′ modification comprises a 2′-OMe, 2′-MOE, 2′-F, 2′-OH, or a combination thereof. In embodiments, the modified nucleic acid comprises a backbone modification, e.g., a phosphorothioate, boranophosphate, methylphosphonate, phosphoramidate (e.g., morpholino phosphoramidate and mesyl phosphoramidate), phosphoramidite, 3′-O-phosphopropylamino, or combination thereof as described herein. Modified nucleic acids are further described herein. In embodiments, the extended oligonucleotide comprises an anchoring oligonucleotide complement that is complementary to the anchoring oligonucleotide. In embodiments, the extended oligonucleotide is bound to the anchoring reagent via hybridization of the anchoring oligonucleotide complement to the anchoring oligonucleotide.

In embodiments, the composition comprises a labeled probe, wherein the labeled probe comprises a detection oligonucleotide. In embodiments, the labeled probe further comprises a detectable label as described herein. In embodiments, the detection oligonucleotide comprises or consists of a sequence as shown in Table 4. In embodiments, the detection oligonucleotide comprises a modified nucleic acid as described herein. In embodiments, the modified nucleic acid comprises a PNA, an LNA, a BNA, a nucleoside comprising a 2′ modification, or a combination thereof. In embodiments, the nucleoside comprising the 2′ modification comprises a 2′-OMe, 2′-MOE, 2′-F, 2′-OH, or a combination thereof. In embodiments, the modified nucleic acid comprises a backbone modification, e.g., a phosphorothioate, boranophosphate, methylphosphonate, phosphoramidate (e.g., morpholino phosphoramidate and mesyl phosphoramidate), phosphoramidite, 3′-O-phosphopropylamino, or combination thereof as described herein. Modified nucleic acids are further described herein. In embodiments, the extended oligonucleotide comprises a detection oligonucleotide complement that is complementary to the detection oligonucleotide. In embodiments, the extended oligonucleotide is bound to the detection oligonucleotide via hybridization of the detection oligonucleotide complement to the detection oligonucleotide.

In embodiments, the invention provides a composition comprising: a capture reagent, an analyte, a detection reagent that comprises an extended oligonucleotide, an anchoring reagent that comprises an anchoring oligonucleotide, and a labeled probe that comprises a detection oligonucleotide. Components of the composition, i.e., the capture reagent, analyte, detection reagent, extended oligonucleotide, anchoring reagent, anchoring oligonucleotide, labeled probe, and detection oligonucleotide are further described herein. In embodiments, the extended oligonucleotide comprises (i) an anchoring oligonucleotide complement that is complementary to the anchoring oligonucleotide; and (ii) a detection oligonucleotide complement that is complementary to the detection oligonucleotide. In embodiments, the extended oligonucleotide is bound to (I) the anchoring reagent on the surface via the anchoring oligonucleotide and (II) the labeled probe via the detection oligonucleotide. In embodiments, an extended oligonucleotide that is bound to both the surface, via the anchoring reagent, and the labeled probe is capable of being detected with a higher sensitivity as compared to an extended oligonucleotide that is bound to the labeled probe and not to the surface. In embodiments, each of the capture reagent and the detection reagent comprises an antibody or antigen-binding fragment thereof. In embodiments, the extended oligonucleotide is extended from a template oligonucleotide of any one of SEQ ID NOs:5, 6, or 16. In embodiments, the anchoring reagent comprises an anchoring oligonucleotide comprising a sequence of any one of SEQ ID NOs:11, 17, and 20-37. In embodiments, the detection oligonucleotide comprises a sequence of any one of SEQ ID NOs:7-10.

### ADDITIONAL EMBODIMENTS

The inventions, e.g., methods, kits, and/or compositions, described herein may further comprise one or more aspects of the following additional embodiments.

### Additional Embodiment (1)

Additional Embodiment (1) includes a method for detecting an analyte, comprising: (a) contacting a template oligonucleotide with a first complex that comprises: (i) the analyte; and (ii) a detection reagent that binds the analyte, wherein the detection reagent comprises a nucleic acid primer; (b) hybridizing the nucleic acid primer to the template oligonucleotide to form a second complex and extending the nucleic acid primer with a polymerase, thereby forming an extended oligonucleotide; (c) contacting the extended oligonucleotide with a single stranded oligonucleotide (SSO) stabilizing agent; and (d) detecting the extended oligonucleotide, thereby detecting the analyte.

In embodiments, the detecting comprises: binding the extended oligonucleotide to one or more labeled probes, wherein each labeled probe comprises (1) a detection oligonucleotide that is capable of binding to the extended oligonucleotide; and (2) a detectable label; and detecting the detectable label. In embodiments, the second complex is bound to a surface.

In embodiments, the SSO stabilizing agent prevents aggregation and/or self-hybridization of the extended oligonucleotide, thereby increasing the availability of the extended oligonucleotide for detection. In embodiments, the extending is performed for at least 5 minutes, at least 10 minutes, at least 15 minutes, at least 30 minutes, at least 45 minutes, at least 1 hour, at least 2 hours, at least 3 hours, at least 4 hours, at least 6 hours, at least 8 hours, at least 10 hours, at least 12 hours, at least 14 hours, at least 16 hours, at least 18 hours, at least 20 hours, at least 22 hours, or at least 24 hours. In embodiments, the SSO stabilizing agent is contacted with the extended oligonucleotide at least 5 minutes, at least 10 minutes, at least 15 minutes, at least 20 minutes, at least 30 minutes, at least 45 minutes, or at least 1 hour after the extending is initiated. In embodiments, the extending is performed for at least 60 minutes, and the SSO stabilizing agent is added about 5 to about 15 minutes after the extending is initiated. In embodiments, the extending is initiated upon contacting the second complex with the polymerase.

In embodiments, the SSO stabilizing agent comprises a DNA binding protein. In embodiments, the DNA-binding protein specifically binds single-stranded DNA. In embodiments, the SSO stabilizing agent is Extreme Thermostable Single-Stranded DNA Binding Protein (ET SSB). In embodiments, the concentration of ET SSB is about 50 ng/mL to about 500 ng/mL, or about 60 ng/mL to about 450 ng/mL, or about 70 ng/mL to about 400 ng/mL, or about 80 ng/mL to about 350 ng/mL, or about 90 ng/mL to about 300 ng/mL, or about 100 ng/mL to about 250 ng/mL, or about 125 ng/mL to about 200 ng/mL, or about 150 ng/mL to about 175 ng/mL.

In embodiments, the SSO stabilizing agent is ET SSB, and the concentration of ET SSB is about 50 ng/mL to about 100 ng/mL immediately after the extending is initiated. In embodiments, the SSO stabilizing agent is ET SSB, and the concentration of ET SSB is about 100 ng/mL to about 150 ng/mL about 5 minutes after the extending is initiated. In embodiments, the SSO stabilizing agent is ET SSB, and the concentration of ET SSB is about 200 ng/mL to about 300 ng/mL about 15 minutes after the extending is initiated.

In embodiments, the method comprising contacting the extended oligonucleotide with the SSO stabilizing agent comprises at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, or at least 10-fold higher assay signal at a given time point, as compared to a method that does not comprise the contacting with the SSO stabilizing agent and is otherwise identical.

### Additional Embodiment (2)

Additional Embodiment (2) includes a method for detecting an analyte, comprising: (a) contacting a template oligonucleotide with a first complex comprising (i) the analyte; (ii) a capture reagent that binds the analyte, wherein the capture reagent is linked to an anchoring reagent that comprises an anchoring oligonucleotide to form a capture reagent-anchoring reagent hybrid, wherein the capture reagent-anchoring reagent hybrid is immobilized or capable of being immobilized to a surface; and (iii) a detection reagent for the analyte and that comprises a nucleic acid primer, (b) hybridizing the nucleic acid primer to the template oligonucleotide to form a second complex and extending the nucleic acid primer with a polymerase, thereby forming an extended oligonucleotide, wherein the extended oligonucleotide comprises an anchoring complement that is capable of binding to the anchoring oligonucleotide; (c) binding the extended oligonucleotide to the anchoring reagent; and (d) detecting the extended oligonucleotide, thereby detecting the analyte.

In embodiments, the detecting comprises: binding the extended oligonucleotide to one or more labeled probes, wherein each labeled probe comprises (1) a detection oligonucleotide that is capable of binding to the extended oligonucleotide; and (2) a detectable label; and detecting the detectable label.

In embodiments, the anchoring reagent is linked to the capture reagent via covalent or non-covalent means. In embodiments, the linking comprises utilizing a crosslinking agent. In embodiments, the crosslinking agent is sulfosuccinimidyl 4-(N-maleimidomethyl)cyclohexane-1-carboxylate (SMCC). In embodiments, the anchoring reagent is linked to the capture reagent via a disulfide bond. In embodiments, the anchoring reagent is linked to the capture reagent via a coupling reaction, e.g., a click reaction.

In embodiments, the capture reagent-anchoring reagent hybrid is directly immobilized onto the surface, e.g., via a covalent linkage. In embodiments, the capture-anchoring reagent is directly immobilized onto the surface via a covalent linkage between the capture reagent portion of the capture reagent-anchoring reagent hybrid and the surface. In embodiments, the capture reagent-anchoring reagent hybrid comprises a first binding partner, which binds to a second binding partner on the surface. In embodiments, the first binding partner is on the capture reagent portion of the capture reagent-anchoring reagent hybrid. In embodiments, the first binding partner is on the anchoring reagent portion of the capture reagent-anchoring reagent hybrid. In embodiments, the first and second binding partners comprise biotin and streptavidin. In embodiments, the first and second binding partners comprise a hapten and a protein capable of binding the hapten. In embodiments, the first binding partner is biotin, and the second binding partner is streptavidin. In embodiments, the anchoring oligonucleotide comprises biotin, the surface comprises streptavidin, and the capture-anchoring reagent hybrid is immobilized to the surface via the biotin on the anchoring oligonucleotide and the streptavidin on the surface.

In embodiments, linking of the anchoring reagent to the capture reagent: (a) provides improved uniformity and control in the preparation of the assay surface; (b) simplifies the assay preparation process by reducing the number of individual components that need to be immobilized onto the surface; and/or (c) provides a consistent ratio of the amount of anchoring and capture reagents present on the surface.

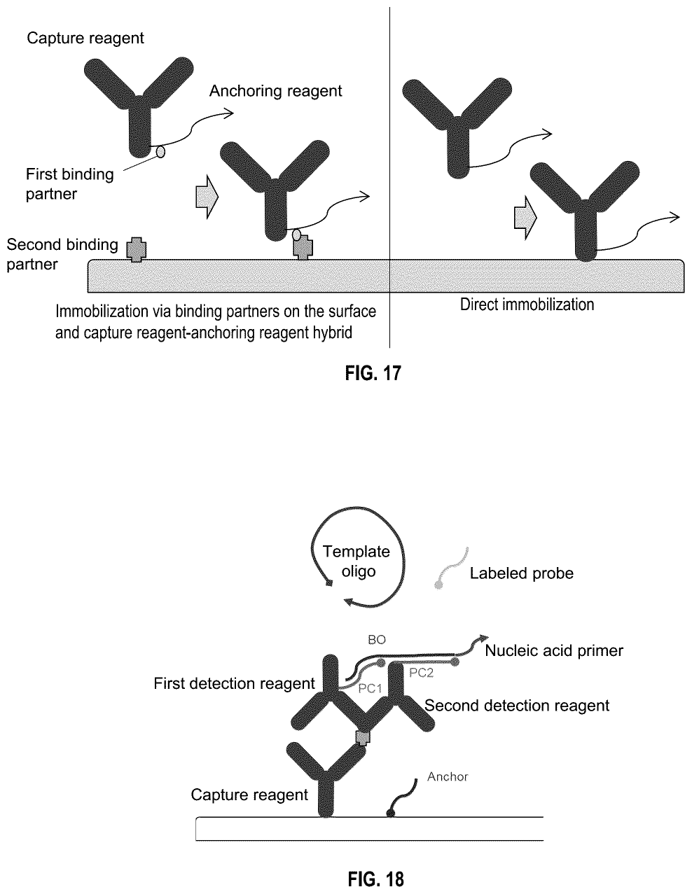

FIG. 17 shows an exemplary illustration of Additional Embodiment (2).

### Additional Embodiment (3)

Additional Embodiment (3) includes a method for detecting an analyte, comprising: (a) contacting a template oligonucleotide with a first complex that comprises: (i) the analyte; (ii) a first detection reagent that binds to the analyte and that comprises a first nucleic acid probe; (iii) a second detection reagent that binds to the analyte and that comprises a second nucleic acid probe; and (iv) a bridging oligonucleotide, wherein a first portion of the bridging oligonucleotide is capable of binding to the first nucleic acid probe and a second portion of the bridging oligonucleotide is capable of binding to the second nucleic acid probe, and wherein the bridging oligonucleotide further comprises a nucleic acid primer; (b) hybridizing the nucleic acid primer to the template oligonucleotide to form a second complex and extending the nucleic acid primer with a polymerase, thereby forming an extended oligonucleotide; and (c) detecting the extended oligonucleotide, thereby detecting the analyte.

In embodiments, the detecting comprises: binding the extended oligonucleotide to one or more labeled probes, wherein each labeled probe comprises (1) a detection oligonucleotide that is capable of binding to the extended oligonucleotide; and (2) a detectable label; and detecting the detectable label. In embodiments, the second complex is bound to a surface.

In embodiments, each of the first and second detection reagents is a detection reagent as described herein, e.g., a protein or polypeptide, antibody or antigen-binding fragment thereof, antigen, ligand, receptor, oligonucleotide, hapten, epitope, mimotope, or aptamer.

In embodiments, the nucleic acid primer of the bridging oligonucleotide is a nucleic acid primer as described herein, e.g., capable of hybridizing to the template oligonucleotide and being extended to form an extended oligonucleotide as described herein. In embodiments, the nucleic acid primer of the bridging oligonucleotide comprises any one of SEQ ID NOs:1-4.

FIG. 18 shows an exemplary illustration of Additional Embodiment (3).

### Additional Embodiment (4)

Additional Embodiment (4) includes a method for detecting an analyte, comprising: (a) contacting a template oligonucleotide with a first complex that comprises (i) the analyte; and (ii) a detection reagent that binds the analyte, wherein the detection reagent comprises a nucleic acid primer; (b) hybridizing the nucleic acid primer to the template oligonucleotide to form a second complex and extending the nucleic acid primer with a polymerase, thereby forming an extended oligonucleotide, wherein the extended oligonucleotide comprises a binding sequence capable of binding to an anchoring reagent, wherein the anchoring reagent is immobilized on a surface, or wherein the first complex further comprises a capture reagent that binds the analyte, wherein the capture reagent is immobilized or capable of being immobilized to the surface, and wherein the anchoring reagent is linked to the capture reagent, and wherein the binding sequence is capable of forming an aptamer or a tertiary oligonucleotide structure, and the anchoring reagent comprises a protein, an antibody, a hapten, or an affinity tag capable of binding to the aptamer or tertiary oligonucleotide structure; (c) binding the binding sequence to the anchoring reagent; and (d) detecting the extended oligonucleotide bound to the surface, thereby detecting the analyte.

In embodiments, the detecting comprises: binding the extended oligonucleotide to one or more labeled probes, wherein each labeled probe comprises (1) a detection oligonucleotide that is capable of binding to the extended oligonucleotide; and (2) a detectable label; and detecting the detectable label. In embodiments, the second complex is bound to a surface.

In embodiments, the binding sequence forms a G-quadruplex, and the anchoring reagent comprises a DNA-binding protein capable of binding the G-quadruplex. In embodiments, the binding sequence that forms the G-quadruplex comprises the formula d(G₃₊N₁₋₇G₃₊N₁₋₇G₃₊N₁₇G₃₊ (SEQ ID NO: 45)), wherein G is guanine and N is any nucleotide. In embodiments, the template oligonucleotide comprises a sequence with the formula d(C₃₊N₁₋₇C₃₊N₁₇C₃₊N₁₋₇C₃₊ (SEQ ID NO: 46)), wherein C is cytosine and N is any nucleotide. In embodiments, the template oligonucleotide comprises the sequence CCCTCCCTCCCTCCC (SEQ ID NO:38).

In embodiments, the binding sequence forms an aptamer, and the anchoring reagent comprises an antibody, hapten, or affinity tag capable of binding to the aptamer. In embodiments, the aptamer-forming binding sequence and its corresponding anchoring reagent are as shown in Table 6.

TABLE 6
Aptamer-Forming Binding Sequences and
Anchoring Reagents

| Binding sequence | Anchoring reagent |
|---|---|
| TCGATTTCCTTAGTTGTCTTCCTTAGTGAG | anti-FLAG M2 |
| (SEQ ID NO: 39) | antibody |
| GCTATGGGTGGTCTGGTTGGGATTGGCCCC | 6X-histidine tag |
| GGGAGCTGGC (SEQ ID NO: 40) | (SEQ ID NO: 44) |
| CCGGCCAAGGGTGGGAGGGAGGGGGCCGG | sulforhodamine B |
| (SEQ ID NO: 41) | |
| AGCGAGGGCGGTGTCCAACAGCGGTTTTTT | digoxin |
| CASCGAGGAGGTTGGCGGTGG | |
| (SEQ ID NO: 42) | |

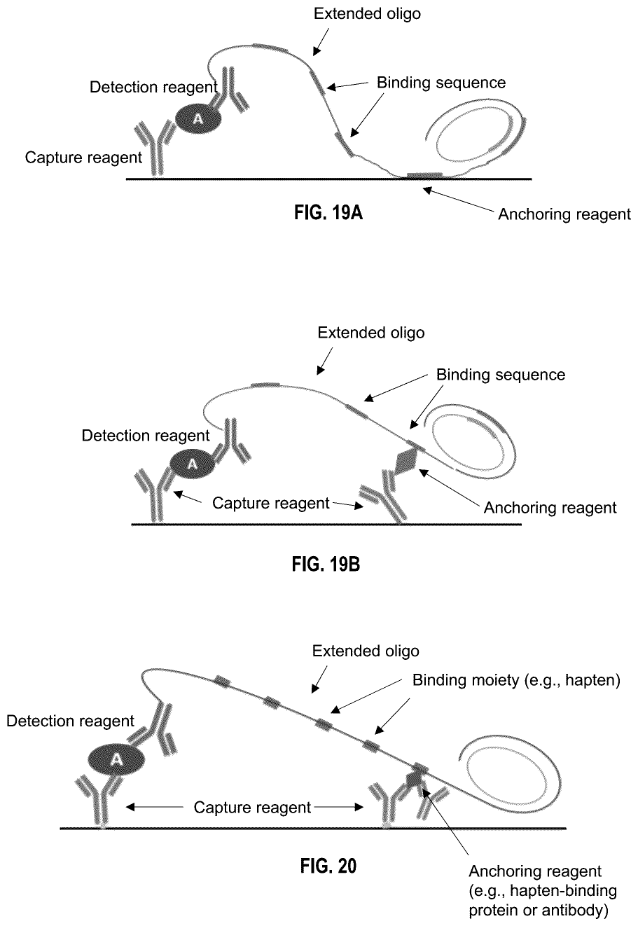

FIGS. 19A and 19B show exemplary illustrations of Additional Embodiment (4).

### Additional Embodiment (5)

Additional Embodiment (5) includes a method for detecting an analyte, comprising: (a) contacting a template oligonucleotide with a first complex that comprises (i) the analyte; and (ii) a detection reagent that binds the analyte, wherein the detection reagent comprises a nucleic acid primer; (b) hybridizing the nucleic acid primer to the template oligonucleotide to form a second complex and extending the nucleic acid primer with a polymerase, thereby forming an extended oligonucleotide, wherein the extended oligonucleotide comprises a binding sequence capable of binding to an anchoring reagent, wherein the anchoring reagent is immobilized on a surface, or wherein the first complex further comprises a capture reagent that binds the analyte, wherein the capture reagent is immobilized or capable of being immobilized to the surface, and wherein the anchoring reagent is linked to the capture reagent, and wherein the binding sequence comprises a binding moiety, and the anchoring reagent comprises a protein or antibody capable of binding the binding moiety; (c) binding the binding sequence to the anchoring reagent; and (d) detecting the extended oligonucleotide bound to the surface, thereby detecting the analyte.

In embodiments, the detecting comprises: binding the extended oligonucleotide to one or more labeled probes, wherein each labeled probe comprises (1) a detection oligonucleotide that is capable of binding to the extended oligonucleotide; and (2) a detectable label; and detecting the detectable label. In embodiments, the second complex is bound to a surface.

In embodiments, the binding moiety is linked to a nucleotide of the binding sequence. In embodiments, the extending comprises incorporating one or more nucleotides linked to the binding moiety into the extended oligonucleotide. In embodiments, the binding sequence comprises at least two binding moieties, and wherein the anchoring reagent is capable of multivalently binding to the at least two binding moieties. In embodiments, the binding moiety comprises a hapten. Non-limiting examples of haptens include digoxigenin, biotin, or dinitrophenol (DNP). In embodiments, the binding sequence comprises at least two haptens, e.g., at least two digoxigenins, biotins, and/or DNPs. In embodiments, the binding moiety comprises digoxigenin, and the anchoring reagent comprises an anti-digoxigenin antibody. In embodiments, the binding moiety comprises biotin, and the anchoring reagent comprises avidin, streptavidin, or an anti-biotin antibody. In embodiments, the binding moiety comprises DNP, and the anchoring reagent comprises an anti-DNP antibody.

FIG. 20 shows an exemplary illustration of Additional Embodiment (5).

### Additional Embodiment (6)

Additional Embodiment (6) includes a method for detecting an analyte, comprising: (a) contacting a template oligonucleotide with a first complex comprising (i) the analyte; (ii) a first detection reagent that binds to the analyte and that comprises a first nucleic acid primer; and (iii) a second detection reagent that binds to the analyte and that comprises a second nucleic acid primer, wherein the template oligonucleotide comprises a first region that is hybridizable with the first nucleic acid primer and a second region that is hybridizable with the second nucleic acid primer; (b) hybridizing the first and second nucleic acid primers to the template oligonucleotide to form a second complex; (c) extending the first nucleic acid primer to form a first extended oligonucleotide, and extending the second nucleic acid primer to form a second extended oligonucleotide; and (d) detecting the first and second extended oligonucleotides, thereby detecting the analyte.

In embodiments, the detecting comprises: binding the first and/or second extended oligonucleotides to one or more labeled probes, wherein each labeled probe comprises (1) a detection oligonucleotide that is capable of binding to the first and/or second extended oligonucleotide; and (2) a detectable label; and detecting the detectable label. In embodiments, the second complex is bound to a surface.

In embodiments, each of the first and second detection reagents is a detection reagent as described herein, e.g., a protein or polypeptide, antibody or antigen-binding fragment thereof, antigen, ligand, receptor, oligonucleotide, hapten, epitope, mimotope, or aptamer. In embodiments, the first nucleic acid primer and the second nucleic acid primer comprise different sequences. In embodiments, each of the first and second nucleic acid primers independently comprises any one of SEQ ID NOs:1-4, provided that the first and second nucleic acid primers are not identical.

In embodiments, the template oligonucleotide comprises one or more connector oligonucleotides that are capable of being ligated to form a circular template, wherein the first region is on a first connector oligonucleotide and the second region is on a second connector oligonucleotide. In embodiments, the method comprises ligating the first and second connector oligonucleotide prior to, during, or after hybridization of the first and/or second nucleic acid primers to the connector oligonucleotides, thereby forming a circular template. In embodiments, the circular template is a template for RCA. In embodiments, the extending comprises RCA.

In embodiments, the method comprising extending both the first and second nucleic acid primers to form first and second extended oligonucleotides, respectively, comprises at least 25%, at least 50%, at least 75%, at least 100%, at least 150%, at least 200%, or at least 300% increase in signal over an otherwise identical method except that only one of the first and second nucleic acid primers is extended.

FIG. 21 shows an exemplary illustration of Additional Embodiment (6).

### Additional Embodiment (7)

Additional Embodiment (7) includes a method for detecting an analyte, comprising: (a) contacting a template oligonucleotide with a first complex comprising (i) the analyte; (ii) a first detection reagent that binds to the analyte and that comprises a first nucleic acid primer; and (iii) a second detection reagent that bind to the analyte and that comprise a second nuclei acid primer, wherein the analyte is present on a surface; (b) hybridizing the first and second nucleic acid primers to the template oligonucleotide to form a second complex; and extending the first and/or the second nucleic acid primer with a polymerase, thereby forming an extended oligonucleotide; and (c) detecting the extended oligonucleotide, thereby detecting the analyte.

In embodiments, the template oligonucleotide comprises one or more connector oligonucleotides that are capable of being ligated to form a circular template, wherein the first region is on a first connector oligonucleotide and the second region is on a second connector oligonucleotide. In embodiments, the method comprises ligating the first and second connector oligonucleotide prior to, during, or after hybridization of the first and/or second nucleic acid primers to the connector oligonucleotides, thereby forming a circular template. In embodiments, the circular template is a template for RCA. In embodiments, the extending comprises RCA.

In embodiments, the detecting comprises: binding the extended oligonucleotide to one or more labeled probes, wherein each labeled probe comprises (1) a detection oligonucleotide that is capable of binding to the extended oligonucleotide; and (2) a detectable label; and detecting the detectable label.

In embodiments, the surface comprises a membrane. In embodiments, the membrane comprises a Western blot membrane. In embodiments, the membrane comprises a nitrocellulose or polyvinylidene difluoride (PVDF) membrane. In embodiments, the method further comprises, prior to the contacting of (a), transferring the analyte from a protein gel to the surface. Methods of transferring an analyte, e.g., a protein, from a protein gel to a membrane, e.g., a Western blot membrane, are known to one of ordinary skill in the art. See, e.g., Mahmood et al., N Am J Med Sci 4(9):429-434 (2012).

In embodiments, the surface further comprises an anchoring reagent that is capable of binding to the extended oligonucleotide, and wherein the method further comprises binding the extended oligonucleotide to the anchoring reagent prior to the detecting of (c). In embodiments, the method further comprises immobilizing the anchoring reagent onto the surface prior to, during, or after the contacting of (a). In embodiments, the anchoring reagent comprises a protein component, and the immobilizing comprises binding the protein component to the surface.

The anchoring reagent of Additional Embodiment (7) may be supplied as a component of a kit described herein. In embodiments, the anchoring reagent is provided pre-immobilized on the surface, e.g., a Western blot membrane described herein. In embodiments, the anchoring reagent is provided in a Western blot transfer buffer, and the anchoring reagent is transferred from the transfer buffer to the surface, e.g., Western blot membrane, prior to the transfer of the analyte onto the surface, e.g., Western blot membrane.

In embodiments, the analyte comprises a protein, and the method comprises contacting the protein with (i) a first detection reagent that binds a post translational modification on the protein and that comprises a first nucleic acid primer; and (ii) a second detection reagent that specifically binds the protein and that comprises a second nucleic acid primer, thereby forming a first complex comprising the analyte, the first detection reagent, and the second detection reagent. Analyte complexes comprising first and second detection reagents are further described herein. In embodiments, the post translational modification on the protein comprises phosphorylation, methylation, acetylation, hydroxylation, deamidation, prenylation, glycosylation, ubiquitylation, AMPylation, ADP-ribosylation, or combination thereof. In embodiments, use of two detection reagents, one that specifically binds the protein and the other that binds the post translational modification, in a method described herein enables highly sensitive and specific detection of post translationally modified proteins.

In embodiments, the surface comprises at least two distinct analytes, and the method is capable of detecting each distinct analyte. In embodiments, each distinct analyte is located at a distinct location on the surface. In embodiments, each distinct analyte is associated with a distinct template oligonucleotide, such that an extended oligonucleotide formed from the first complex binds to a distinct anchoring reagent. In embodiments, each distinct template oligonucleotide, and therefore each distinct analyte, is associated with a unique detectable label, e.g., fluorescent label, quantum dot, or enzymatic activity, allowing the at least two distinct analytes to be detected independently.

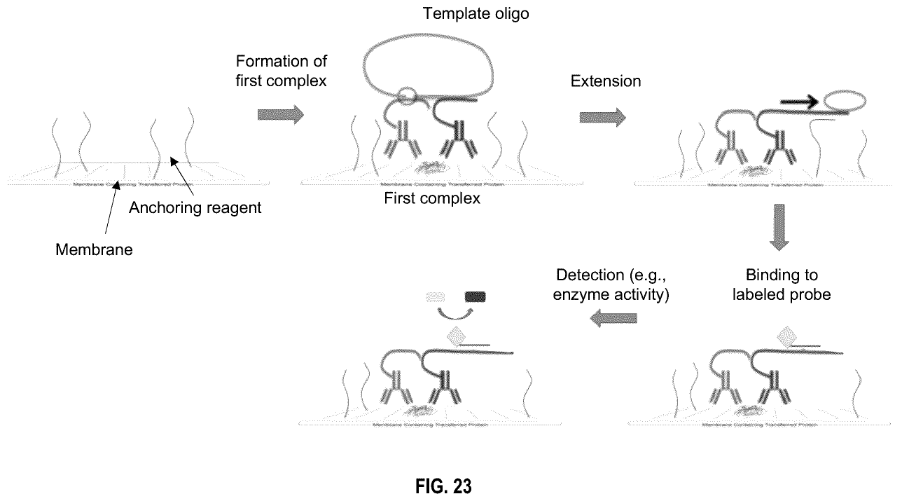

FIG. 23 shows an exemplary illustration of Additional Embodiment (7).

### Additional Embodiment (8)

Additional Embodiment (8) includes a method for detecting an analyte, comprising: (a) contacting a template oligonucleotide with a first complex that comprises: (i) the analyte; and (ii) a detection reagent that binds the analyte, wherein the detection reagent comprises a nucleic acid primer; (b) hybridizing the nucleic acid primer to the template oligonucleotide to form a second complex and extending the nucleic acid primer with a polymerase, thereby forming an extended oligonucleotide, wherein the extended oligonucleotide is capable of forming a secondary structure comprising a detectable enzymatic activity; and (c) detecting the detectable enzymatic activity, thereby detecting the analyte.

In embodiments, the second complex is bound to a surface.

In embodiments, the secondary structure comprises an aptamer. In embodiments, the aptamer comprises enzymatic activity. In embodiments, the aptamer comprises enzymatic activity in the presence of an activator compound. In embodiments, the enzymatic activity is peroxidase activity, and the activator compound is hemin. Aptamers with peroxidase activity in the presence of hemin is described, e.g., in Liu et al., Bull. Chem. Soc. Jpn. 82(1):99-104 (2009). In embodiments, the aptamer comprises the sequence ATTGGGAGGGATTGGGTGGG (SEQ ID NO:43).

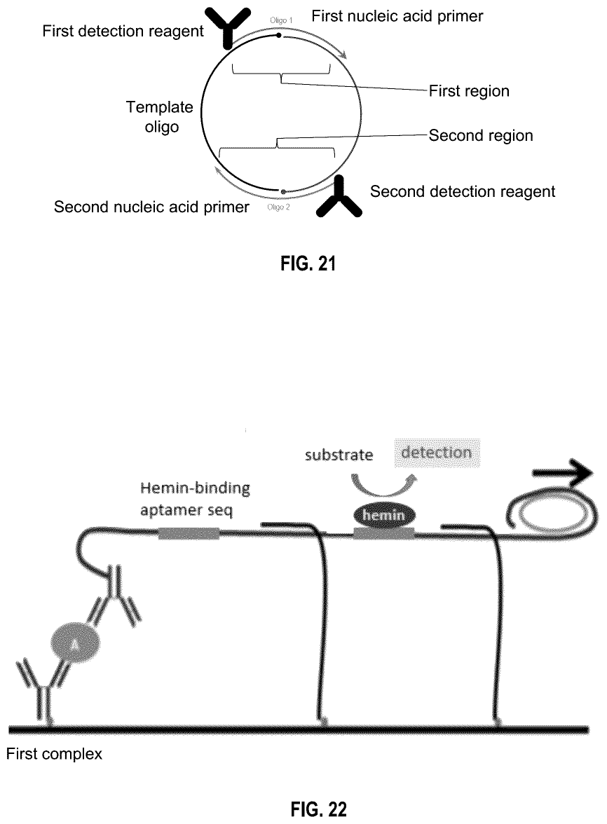

FIG. 22 shows an exemplary illustration of Additional Embodiment (8).

### Sequences

In embodiments, the invention provides an oligonucleotide of any one of SEQ ID NOs:1-37. In embodiments, the invention provides an oligonucleotide of any one of SEQ ID NOs:1-18. In embodiments, the invention provides an oligonucleotide comprising SEQ ID NO:11, SEQ ID NO:17, or any one of SEQ ID NOs:20-37. In embodiments, the invention provides an oligonucleotide comprising any one of SEQ ID NOs:7-10, any one of SEQ ID NOs:12-15, or SEQ ID NO:19. In embodiments, the invention provides an oligonucleotide consisting of SEQ ID NO:11, SEQ ID NO:17, or any one of SEQ ID NOs:20-37. In embodiments, the invention provides an oligonucleotide consisting of any one of SEQ ID NOs:7-10, any one of SEQ ID NOs:12-15, or SEQ ID NO:19.

TABLE 7
Sequences

| | Sequence | | SEQ |
|---|---|---|---|
| Description | Name | Sequence | ID No |
| Nucleic acid primer | PP2-15 | GACAGAACTAGACAC | 1 |
| Nucleic acid primer | | ACAGAACTAGACAC | 2 |
| Nucleic acid primer | | GACAGAACTAGACA | 3 |
| Nucleic acid primer | | TGCACAGCTCGACGC | 4 |
| Template oligonucleotide | Circ61 | GTTCTGTCATATTTCAGTGAATGCGAGTCCG | 5 |
| | | TCTAAGAGAGTAGTACAGCAAGAGTGTCTA | |
| Template oligonucleotide | | GCTGTGCAATATTTCAGTGAATGCGAGTCCG | 6 |
| | | TCTAAGAGAGTAGTACAGCAAGAGCGTCGA | |
| Detection oligonucleotide | D10A | mG+A+G+T+C+CmGmUmCmU | 7 |
| Detection oligonucleotide | D10B | C+A+G+T+G+AA+TGC | 8 |
| Detection oligonucleotide | LNA-10/6 | G+A+G+T+C+C+GTCT | 9 |
| Detection oligonucleotide | D10+5L(A) | G+A+G+T+C+CGTCT | 10 |
| Anchoring oligonucleotide | | mU+AmGmUmA+C+AmGmC | 11 |
| Detection oligonucleotide with modifiers | D10A | mG+A+G+T+C+CmGmUmCmU/iAmMC6T/ | 12 |
| | | iSp18/iAmMC6T/iSp18/3AmMO/ | |
| Detection oligonucleotide with modifiers | D10B | C+A+G+T+G+AA+TGC/iAmMC6T/iSp18/ | 13 |
| | | iAmMC6T/iSp18//3AmMO/ | |
| Detection oligonucleotide with modifiers | LNA-10/6 | G+A+G+T+C+C+GTCT/iAmMC6T/iSp18/ | 14 |
| | | iAmMC6T/iSp18/3AmMO/ | |
| Detection oligonucleotide with modifiers | D10+5L(A) | G+A+G+T+C+CGTCT/iAmMC6T/iSp18/ | 15 |
| | | iAmMC6T/iSp18/3AmMO/ | |
| Template oligonucleotide with 5′ | Circ61 | /5Phos/GTTCTGTCATATTTCAGTGAATGC | 16 |
| phosphorylation | | GAGTCCGTCTAAGAGAGTAGTACAGCAAGAG | |
| | | TGTCTA | |
| Anchoring oligonucleotide with 3′ biotin | A9+3L6OM | mU+AmGmUmA+C+AmGmC/3Bio/ | 17 |
| Nucleic acid primer with 5′ thiol | PP2-15 | 5ThioMC6-D/GACAGAACTAGACAC | 18 |
| Detection oligonucleotide with modifiers | Detect23 | CAGTGAATGCGAGTCCGTCTAAG/iAmMC6T/ | 19 |
| | | iSp18/iAmMC6T/iSp18/3AmMO/ | |
| Anchoring oligonucleotide with 3′ thiol | A12 | GTA GTA CAG CAA /3ThioMC3-D/ | 20 |
| Anchoring oligonucleotide with 3′ thiol | A25 | AAG AGA GTA GTA CAG CAG CCG | 21 |
| | | TCA A/3ThioMC3-D/ | |
| Anchoring oligonucleotide with 3′ thiol | LNA9-1 | T+AGTACAGC/3ThioMC3-D/ | 22 |
| Anchoring oligonucleotide with 3′ thiol | LNA9-2 | T+AGTA+CAGC/3ThioMC3-D/ | 23 |
| Anchoring oligonucleotide with 3′ thiol | LNA9-3 | T+AGTA+C+AGC/3ThioMC3-D/ | 24 |
| Anchoring oligonucleotide with 3′ thiol | LNA9-6 | T+A+G+T+A+C+AGC /3ThioMC3-D/ | 25 |
| Anchoring oligonucleotide with 3′ thiol | LNA9-7 | T+A+G+T+A+C+A+GC /3ThioMC3-D/ | 26 |
| Anchoring oligonucleotide with 3′ thiol | LNA9-8 | T+A+G+T+A+C+A+G+C /3ThioMC3-D/ | 27 |
| Anchoring oligonucleotide with 3′ thiol | LNA9-9 | +T+A+G+T+A+C+A+G+C/3ThioMC3-D/ | 28 |
| Anchoring oligonucleotide with 3′ biotin | A10 | GTA GTA CAG C/3Bio/ | 29 |
| Anchoring oligonucleotide with 3′ biotin | A12 | GTA GTA CAG CAA /3Bio/ | 30 |
| Anchoring oligonucleotide with 3′ biotin | A25 | AAG AGA GTA GTA CAG CAG CCG | 31 |
| | | TCA A/3Bio/ | |
| Anchoring oligonucleotide with 3′ biotin | A12-OM | mGmTmAmGmTmAmCmAmGmCmAmA /3Bio/ | 32 |
| Anchoring oligonucleotide with 3′ biotin | A9+3L | T+AGTA+C+AGC/3Bio/ | 33 |
| Anchoring oligonucleotide with 3′ thiol | A9+3L6OM | mU+AmGmUmA+C+AmGmC/3ThioMC3-D/ | 34 |
| | No Peg | | |
| Anchoring oligonucleotide with 3′ thiol | A9+3L6OM | mU+AmGmUmA+C+AmGmC/iSp18// | 35 |
| and one Peg6 spacer | 1Peg | 3ThioMC3-D/ | |
| Anchoring oligonucleotide with 3′ thiol | A9+3L6OM | mU+AmGmUmA+C+AmGmC/iSp18// | 36 |
| and two Peg6 spacer | 2Peg | iSp18//3ThioMC3-D/ | |
| Anchoring oligonucleotide with 3′ biotin | A9+4L | T+A+G+TA+CAGC /3Bio/ | 37 |

All references cited herein, including patents, patent applications, papers, textbooks and the like, and the references cited therein, to the extent that they are not already, are hereby incorporated herein by reference in their entirety.

### EXAMPLES

### Example 1. General Protocol for Sandwich Immunoassay

An ECL-based detection assay according to embodiments herein is performed as follows:

A detection antibody is modified by the addition of a nucleic acid primer using oligonucleotide-polypeptide conjugation techniques known to one of ordinary skill in the art, e.g., as described in WO 2020/180645. Streptavidin-coated plate wells of a multi-well plate are coated with biotinylated anchoring oligonucleotides and biotinylated capture antibody, followed by washing. A blocking solution and a sample containing target analyte are then added to the wells. After incubating at room temperature, the wells are washed. A solution containing the nucleic acid primer-conjugated detection antibodies is added to each well (25 μL per well), and incubated with shaking for 1-2 hours. A ligation mix, which includes (i) template oligonucleotide (4 nM), ligation buffer, ATP (1 mM), and T4 DNA ligase (0.15 U/pL), is then added to each well. The plate is incubated with the ligation mix for 30 minutes at room temperature, washed to remove excess template oligonucleotides, and incubated for 1.5 hours at 37° C. with a rolling circle amplification (RCA) mixture that contains RCA buffer, dNTP (250 pM of each), and Phi29 DNA polymerase (0.125 U/ml). Following incubation with the RCA mixture, the plate is washed, then incubated for 30 minutes at 37° C. with a detection mixture that contains 20 mM Tris. 1 mM EDTA, 250 mM NaCl, 0.01% TRITON, BSA (200 μg/mL), TWEEN 20 (0.05%), and a mixture of labeled probes comprising detection oligonucleotides (6.25 nM). Following incubation with the detection mixture, the plate is washed, and 150 μL MSD read buffer is added. The plate is read immediately following addition of read buffer on an MSD SECTOR© 6000 Reader (plates and reader supplied by Meso Scale Discovery, Rockville, MD, USA).

### Example 2. Improved Detection Oligonucleotides

Conventional detection oligonucleotides (i.e., that do not contain any modified nucleotides) of labeled probes used in ECL-based detection assays inhibit activity of Phi29 DNA polymerase during RCA, resulting in lower assay sensitivity and longer assay run time. To mitigate the polymerase inhibition issue, assays are typically performed without the labeled probe during the extending step, and the labeled probe is added in a separate step following the extending as described in the protocol of Example 1. See also, e.g., WO 2014/165061; WO 2014/160192; and WO 2015/175856. This two-part extension and detection increases assay performance but also increases assay run time.

Improved detection oligonucleotides were developed to be shorter (less than 20 nucleotides in length) as compared to a conventional detection oligonucleotide (greater than 20 nucleotides in length) and incorporated locked nucleic acid (LNA) residues. Such detection oligonucleotides were discovered not to inhibit the DNA polymerase, thereby generating higher ECL assay signal. Shorter detection oligonucleotides can lower assay development complexity as compared to a conventional detection oligonucleotide and can be used to generate higher assay signal even when provided at a lower concentration as compared to a conventional detection oligonucleotide. Further, when using shorter detection oligonucleotides, more copies of the detection oligonucleotide can bind to an extended oligonucleotide as compared to the conventional detection oligonucleotide, thereby increasing assay signal.

The improved detection oligonucleotides were synthesized and purified by FPLC prior to use in the assays describe herein. For FPLC, a HiTrap Q HP 1 mL anion exchange column was—used with the AKTA™ PURE 25 system. Two gradients were tested: Method 1—0-800 mM NaCl gradient; Method 2—400-700 mM NaCl gradient. The eluted fractions were analyzed on a 15% Urea TBE Gel with an IDT 20/100 DNA Ladder, then visualized with SYBR Gold Stain.

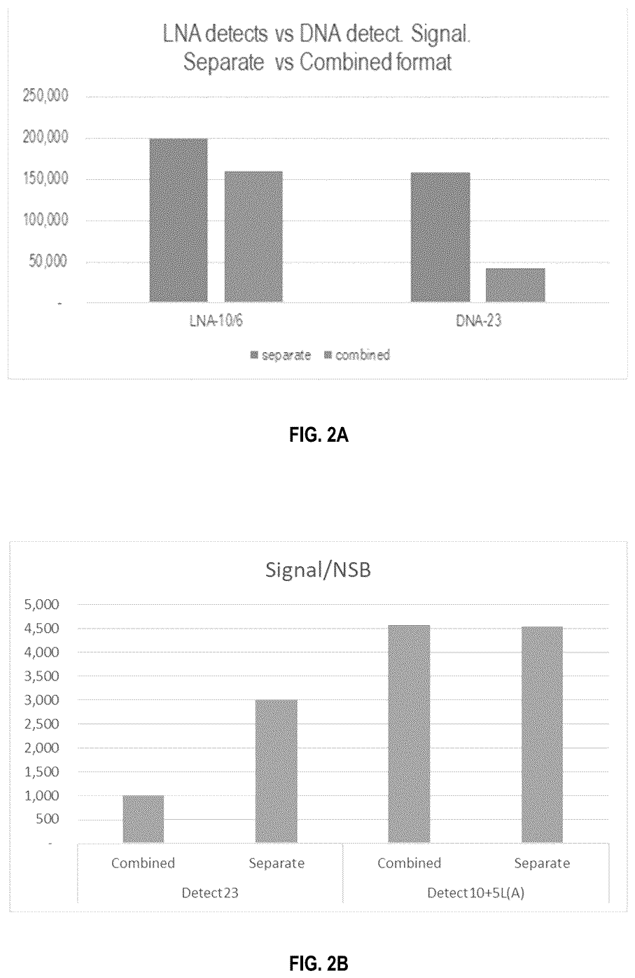

The improved detection oligonucleotides were employed in comparative assays versus conventional detection oligonucleotides following a simplified version of the protocol described in Example 1. In this simplified protocol, instead of employing a biotinylated capture antibody that attaches to the streptavidin-coated plate surface to form a sandwich complex (which includes the capture antibody, target analyte, and the detection antibody conjugated with nucleic acid primer), a biotinylated nucleic acid primer oligonucleotide was directly attached to the streptavidin-coated surface, for example, at 33 fM concentration, comprising 10⁶ molecules of the primer oligonucleotide per well. FIG. 2A shows representative results of a comparative assay performed with a conventional detection oligonucleotide of 23 nucleotides in length and without any modified nucleic acids (labeled in FIG. 2A as “DNA-23”) and an improved detection oligonucleotide according to the invention, of 10 nucleotides in length and includes 6 LNAs (labeled in FIG. 2A as “LNA-10/6”). Each of the two detection oligonucleotides was added at a concentration of 6.25 nM, either during the extending step (“combined” format) or in a separate step following the extension (“separate” format). The results show that in both the separate and combined formats, the improved detection oligonucleotide provided higher assay signal.

FIG. 2B shows representative results of a further comparison of the conventional 23-nucleotide detection oligonucleotide (labeled in FIG. 2B as “Detect23”) with an improved detection oligonucleotide according to the invention, comprising 10 nucleotides in length and includes 5 LNAs (labeled in FIG. 2B as “Detect10+5L(A)”). The signal to non-specific background (NSB) ratio was significantly better in both the “combined” and “separate” formats for the improved detection oligonucleotide as compared to the conventional detection oligonucleotide.

Sequences of the detection oligonucleotides are provided below (shown with modifiers for conjugation to detectable labels). Each oligonucleotide was conjugated with three SULFO-TAG®-NHS.

Sequences of detection oligonucleotides used in this example:

### 23-Nucleotide Conventional Detection Oligonucleotide (DNA-23 or Detect23):

(SEQ ID NO: 19)
CAGTGAATGCGAGTCCGTCTAAG/iAmMC6T/iSp18/iAmMC6T/
iSp18/3AmMO/

10-Nucleotide Detection Oligonucleotide with 6 LNAs (LNA-10/6):

(SEQ ID NO: 14)
G+A+G+T+C+C+GTCT/iAmMC6T/iSp18/iAmMC6T/iSp18/
3AmMO/

10-Nucleotide Detection Oligonucleotide with 5 LNAs (Detect10+5L(A)):

(SEQ ID NO: 15)
G+A+G+T+C+CGTCT/iAmMC6T/iSp18/iAmMC6T/iSp18/3AmMO/

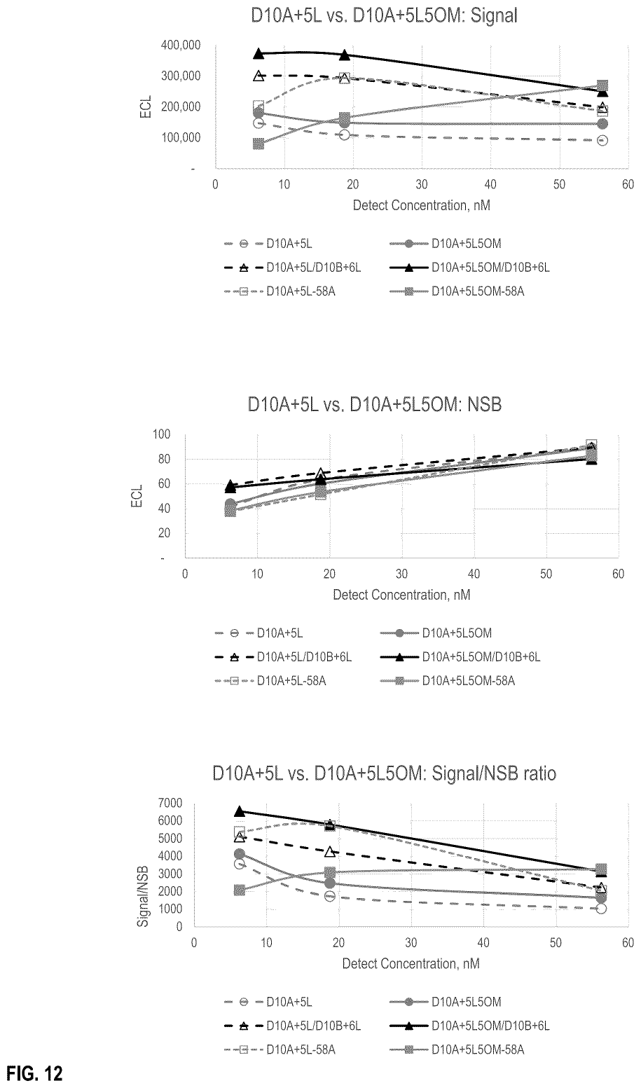

A benchmark assay comparing detection oligonucleotides with various combinations of LNAs and/or 2′-OMe nucleotides demonstrated that including 2′-OMe nucleotides aided in reducing interference between polymerase and the detection oligonucleotides in the reaction mixture. The results of the benchmark assay are shown in FIG. 12. The detection oligonucleotides and template oligonucleotides represented in FIG. 12 are as follows:
D10A+5L: 10-nucleotide (nt) detection oligonucleotide with 5 LNAs; used with a 61-nt template
D10A+5L5OM: 10-nt detection oligonucleotide with 5 LNAs and 5 2′-OMe nucleotides; used with a 61-nt template
D10A+5L-58A: 10-nt detection oligonucleotide with 5 LNAs; used with a 58-nt template
D10A+5L5OM-58A: 10-nt detection oligonucleotide with 5 LNAs and 5 2′-OMe nucleotides; used with a 58-nt template
D10A+5L/D10B+6L: Mixture of D10A+5L and D10B+6L; used with a 61-nt template D10A+5L5OM/D10B+6L: Mixture of D10A+5L5OM and D10B+6L; used with a 61-nt template

The assays performed with D10A+5L-58A and D10A+5L5OM-58A detection oligonucleotides utilized a 58-nt template oligonucleotide that contained three copies of a “D10A” sequence, while the assays performed with D10A+5L, D10A+5L5OM, D10A+5L/D10B+6L, and D10A+5L5OM/D10B+6L detection oligonucleotides utilized a 61-nt template oligonucleotide that contained only one copy of the D10A sequence and one copy of a “D10B” sequence. As shown in FIG. 12, D10A+5L5OM (containing 2′-OMe nucleotides) had a higher signal/non-specific binding (NSB) ratio as compared to D10A+5L (no 2′-OMe nucleotides), and D10A+5L5OM/D10B+6L (containing 2′-OMe nucleotides) had a higher signal/NSB ratio as compared to D10A+5L/D10B+6L (no 2′-OMe nucleotides) for all detection oligonucleotide concentrations tested. When D10A+5L was tested with the 58-nt template (“D10A+5L-58A”), the signal/NSB ratio dependence on the detection oligonucleotide concentration was similar to that observed on the 61-nt template, with a gradual decline of the ratio at increased detect concentration, possibly due to the increased interference with polymerase. When D10A+5L5OM5L was tested on the 58-nt template (“D10A+5L5OM-58A”), both signal and signal/NSB ratio increased with increased detection oligonucleotide concentration, demonstrating reduced interference with polymerase as compared to D10A+5L. However, with the 58-nt template oligonucleotide, the signal and signal/NSB ratios were lower for D10A+5L5OM at 6.2 and 19 nM concentrations of detection oligonucleotide, and higher only at 56 nM detection oligonucleotide concentration, as compared to D10A+5L at the same concentrations. The sequence differences between the 58-nt and 61-nt templates and the presence of the triplicate repeat sequence in the 58-nt template may have contributed to the observed difference in signal and signal/NSB ratio between the two templates tested with similar pairs of detect oligonucleotides.

### Example 3. Cleavage of Template Oligonucleotide

Termination of the extension reaction in the assays described herein by cleaving the template oligonucleotide was tested for assay end-point stability and robustness to variations in time and temperature.

The following restriction enzymes of different type and having different length recognition sites were evaluated: AvaII, BstMutI, DdeI, HinfI, Hpy188I, NciI, Sau96I, ScrFI, TspRI (5 nt); AluI, BfaI, CviAII, CviKI01, CviQI, DpnII, FatI, HpaII, HpyCH4IV, MboI, MluCI, MseI, MspI, NlaIII, RsaI, Sau3AI, TaqI-v2 (4 nt); and StuI, Apo I, AseI, AvaI, BsaAI, BsmI, BsrI, PmlI, PvuI, SmaI, XmaI (6 nt).

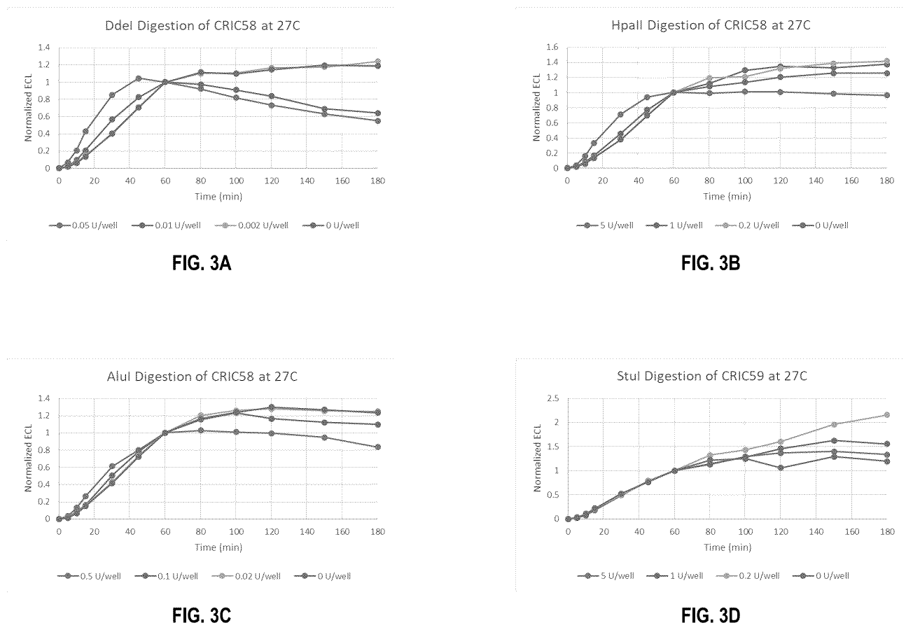

The restriction enzymes DdeI, AluI, HpaII, and StuI were tested in the simplified assay protocol as described in Example 2. The assay further employed a 58-nucleotide template oligonucleotide with DdeI, HpaII, or AluI restriction sites, and a 59-nucleotide template oligonucleotide with a StuI restriction site. Amplification was performed with Phi29 polymerase (0.5 μg/mL) at 27° C. for 1 hour. 6.25 nM Detect10+5L(A) detection oligonucleotide and DdeI, HpaII, AluI, or StuI were added at the same time as the polymerase. Amplification was stopped by addition of PBS with 10 mM EDTA at each time point. MSD® Read Buffer A was used to generate ECL assay signal. Results are shown in FIGS. 3A-3D for DdeI, HpaII, AluI, and StuI, respectively. All restriction enzymes except for StuI showed clear ability to terminate the assay.

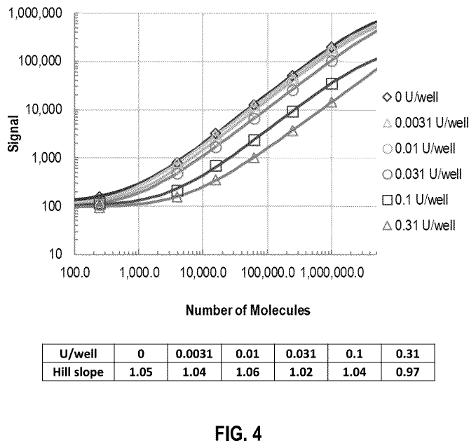

A further experiment was performed with DdeI cleavage of the template oligonucleotide to evaluate calibration curve and measure termination kinetics. For the calibration curve, the same assay as above was performed with a 61-nucleotide template oligonucleotide, 0 to 0.3 U/well DdeI, at 27° C. for 0.5 hours. The calibration curve is shown in FIG. 4 and indicates termination rates did not affect Hill slope, which remained stable with varying DdeI concentration.

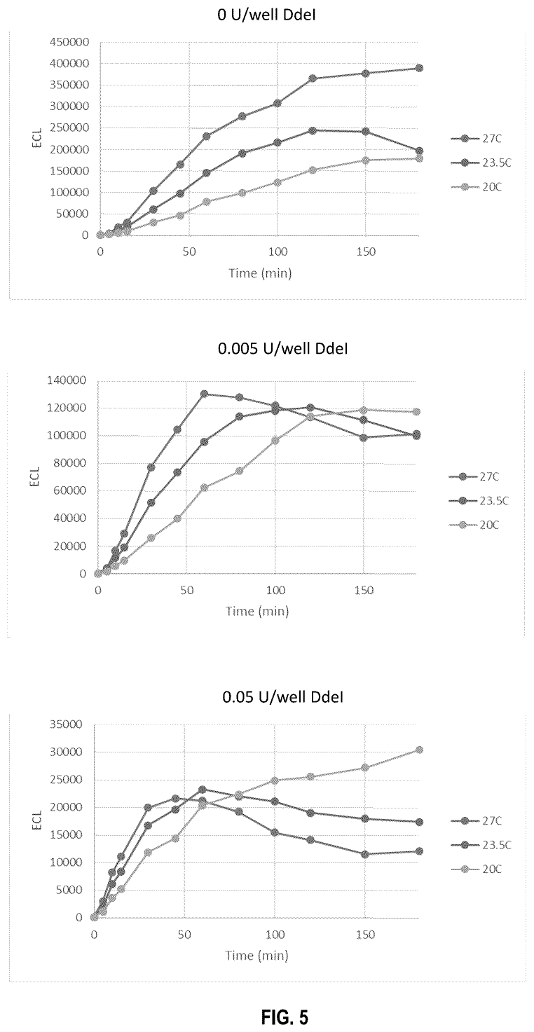

For measuring termination kinetics, the same assay as above was performed with a 58-nucleotide template oligonucleotide with 0, 0.005, or 0.05 U/well DdeI at 20° C., 23.5° C., and 27° C. for time points up to 180 minutes. The termination kinetics data in FIG. 5 show that in contrast to the “No enzyme” condition, in the presence of restriction enzyme there is a time point at which signal intensity is similar for all three temperatures tested. The time at which signal is equivalent at different temperatures is dependent on the DdeI concentration. For 0.005 U/well DdeI concentration, similar signal could be achieved at around 120 minutes time point for all three conditions: 20° C., 23.5° C. and 27° C., while for 0.05 U/well DdeI concentration, the equal signal could be reached around the 60 minutes time point. This observation suggests that at different enzyme concentration, there is a time window within which the signal generation is not temperature dependent.

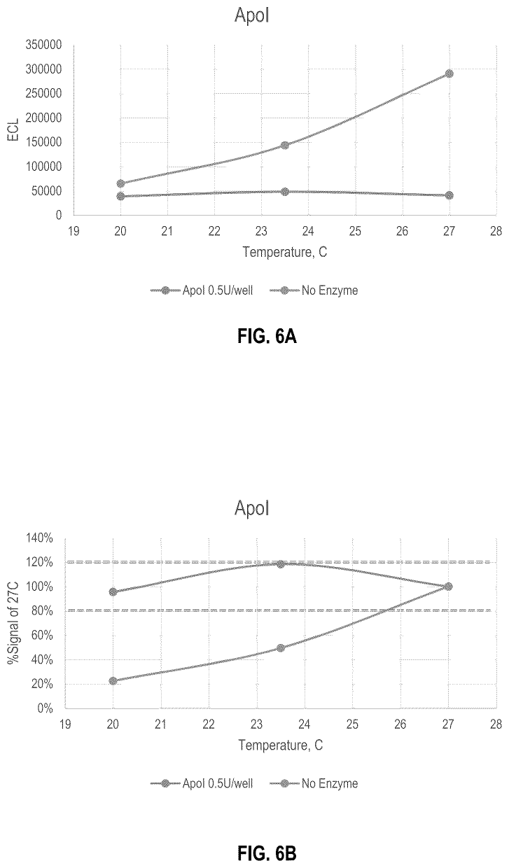

Temperature independent signal generation could be reached with specific enzyme concentration at the amplification step. FIG. 6A shows significant signal dependence on the temperature in the absence of enzyme, with a change in signal from about 60,000 ECL at 20° C. to 150,000 ECL at 23.5° C., and to 300,000 ECL at 27° C., while in the presence of 0.5 U/well of ApoI, the signal remained within 45,000-50,000 ECL at all three temperatures. FIG. 6B shows the same results with signal normalized to 1 hour at 27° C. Without termination, signal varies from 20 to 100%, while with ApoI in the reaction mix, overall signal difference between temperatures stays within 80-120% interval.

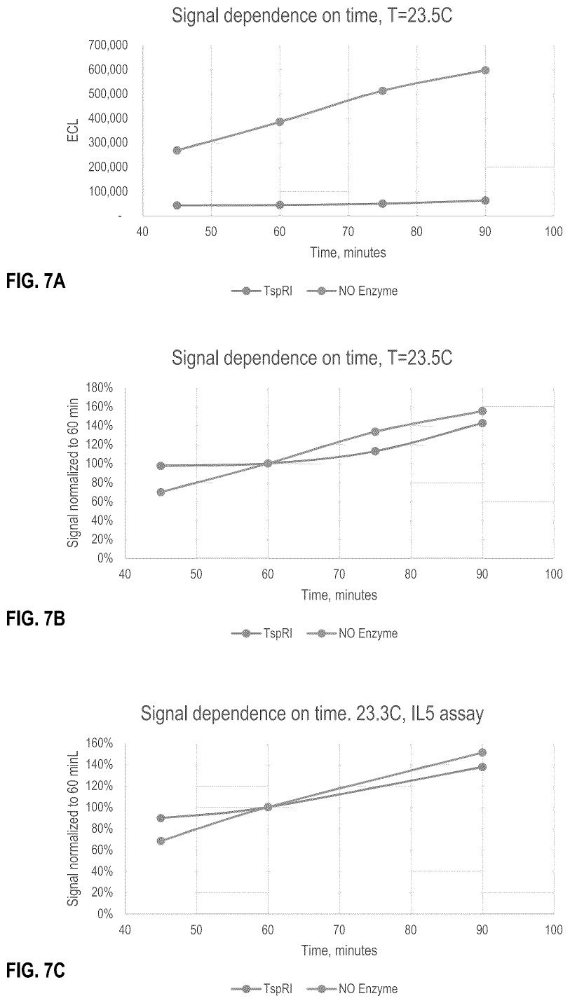

In addition, termination of the amplification reaction by template cleavage also contributes towards achieving time-independent signal generation. Amplification reactions were tested at three different temperatures, 20° C., 23.5° C. and 27° C., in the presence and the absence of enzyme (exemplary results with TspRI are shown in FIGS. 7A-7C), and signal generation measured at several time points: 45 min, 60 min, 75 min, 90 min. The results demonstrate that at all temperatures tested, signal is less dependent on time with termination compared to the amplification without termination. An example of amplification results generated at 23.5° C. is shown in FIGS. 7A and 7B: no signal difference was observed at 45 min compared to the 60 min time point with TspRI termination, and about 30% difference was observed in the absence of enzyme between the 45 min and 60 min time points. In addition, 13% vs 33%, and 43% vs 55% signal changes were observed for the terminated reaction vs not-terminated reaction at 75 min and 90 min time points, respectively. A “full” version of the two-antibody sandwich immunoassay (with IL-5 as analyte) according to Example 1 was performed on an MSD® streptavidin-coated plate with and without TspRI termination, at 23.3° C. (FIG. 7C). The results with the simplified immunoassay as described in Example 2 (FIG. 7B) and the full version of the immunoassay (FIG. 7C) are similar, confirming improved signal independence of time with termination compared to no termination.

### Example 4. Improved Anchoring Oligonucleotides

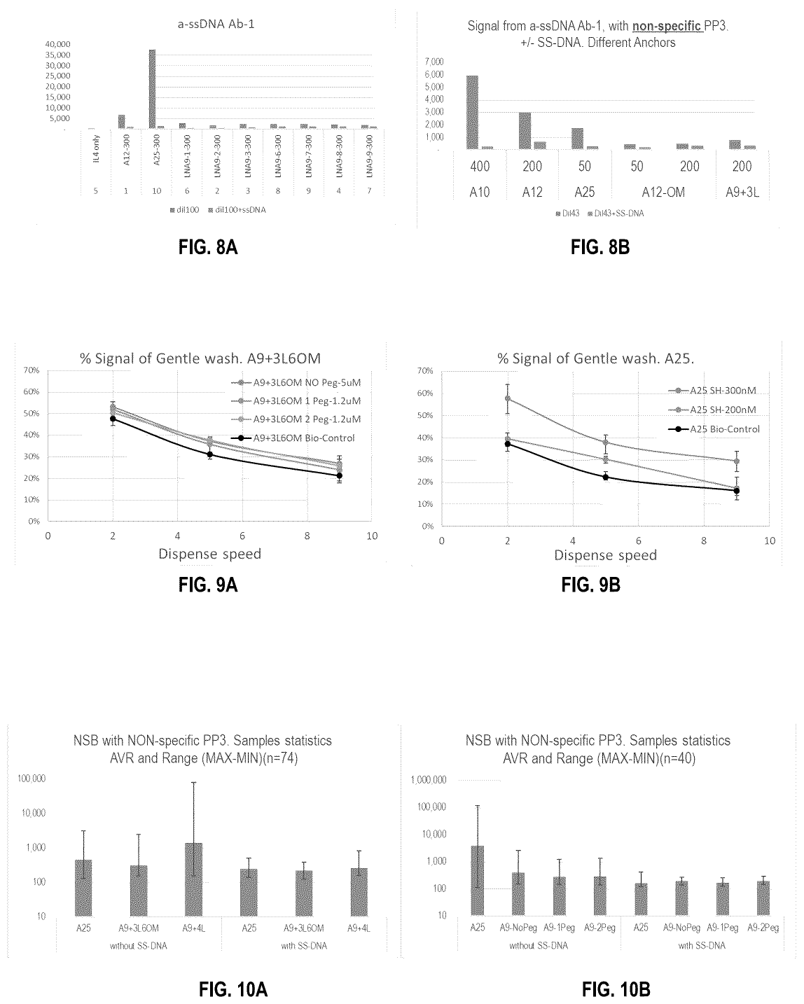

Shorter anchoring oligonucleotides can lower assay development complexity as compared to longer anchoring oligonucleotides. Therefore, anchoring reagents comprising anchoring oligonucleotides of 12 nucleotides in length (“12-mer”, e.g., A12 designations in FIGS. 8A and 8B) as well as 9-mer oligonucleotides (e.g., A9 designations in FIGS. 8-11) with locked nucleic acids (LNA) and/or 2′-O-methylated (2′-OMe or OM) nucleic acids were tested in assays as described in Example 2 against longer, conventional anchoring oligonucleotides of 25 nucleotides in length.

FIG. 8A reveals that the shorter anchoring oligonucleotides were found to lower background signal when used at the same concentration as longer oligonucleotides). However, as revealed in FIG. 8B, a higher coating concentration was required for the shorter anchoring oligonucleotides, which increased background. In FIG. 8A, ECL signal is indicated on the y-axis of the graph and the various anchoring oligonucleotides are denoted on the x-axis. “IL-4 only” indicates assay without anchoring oligonucleotide while “A12-300” and “A25-300” denote 12-mer and 25-mer anchoring oligonucleotides without any modified nucleotides, respectively, at a concentration of 300 nM. LNA9-1-300, LNA9-2-300, etc. denote anchoring oligonucleotides that are 9 nucleotides in length and that have 1 or 2 locked bases, respectively. It was discovered that incorporation of locked nucleic acids and/or 2′-O-methylated (2′-OMe) nucleic acids into the anchoring oligonucleotides reduced the required coating concentration and further reduced the background (FIGS. 8A and 8B).

The anchoring oligonucleotides were immobilized on the surface either via binding of a biotin moiety on the anchoring oligonucleotide to a streptavidin on the surface, or via conjugation to the surface via a thiol moiety on the anchoring oligonucleotide. Anchoring oligonucleotides comprising a thiol moiety further included a PEG-spacer between the anchoring oligonucleotide and the thiol group. The results for modified short anchors are shown in FIG. 9A, and for longer 25-mer oligonucleotides in FIG. 9B FIGS. 9A-9B show the stabilization of the ECL signal, expressed as the percentage of ECL signal retained as washer speed is increased. In FIG. 9A the shorter A9+3L6OM Anchors, containing LNA and 2′-OMe bases, demonstrated similar stability to the A25 DNA based Anchors in FIG. 9B.

Sample matrix interferences were reduced when short, modified anchoring oligonucleotides were used as compared to a longer, conventional anchoring oligonucleotide. This reduction in sample matrix interference was both a reduction in the average non-specific signal and the range of non-specific signals across samples (FIGS. 10A and 10B). This lower average and range of non-specific signals within a sample set allows for improved real world sample sensitivities. Human samples background and its variability on the surface with conventional longer anchors and short modified anchors immobilized on the surface via binding of a biotin moiety are shown in FIG. 10A, and with anchors immobilized via a thiol moiety in FIG. 10B.

### Example 5. Combination of Modified Anchoring Oligonucleotide, Modified Detection Oligonucleotide, and Template Oligonucleotide Cleavage

The improved anchoring oligonucleotide comprising LNAs and 2′-OMe modified nucleic acids, improved detection oligonucleotide comprising LNAs, and template oligonucleotide cleavage with DdeI as described in Example 2-4 were tested individually and in combination for assay wash stability.

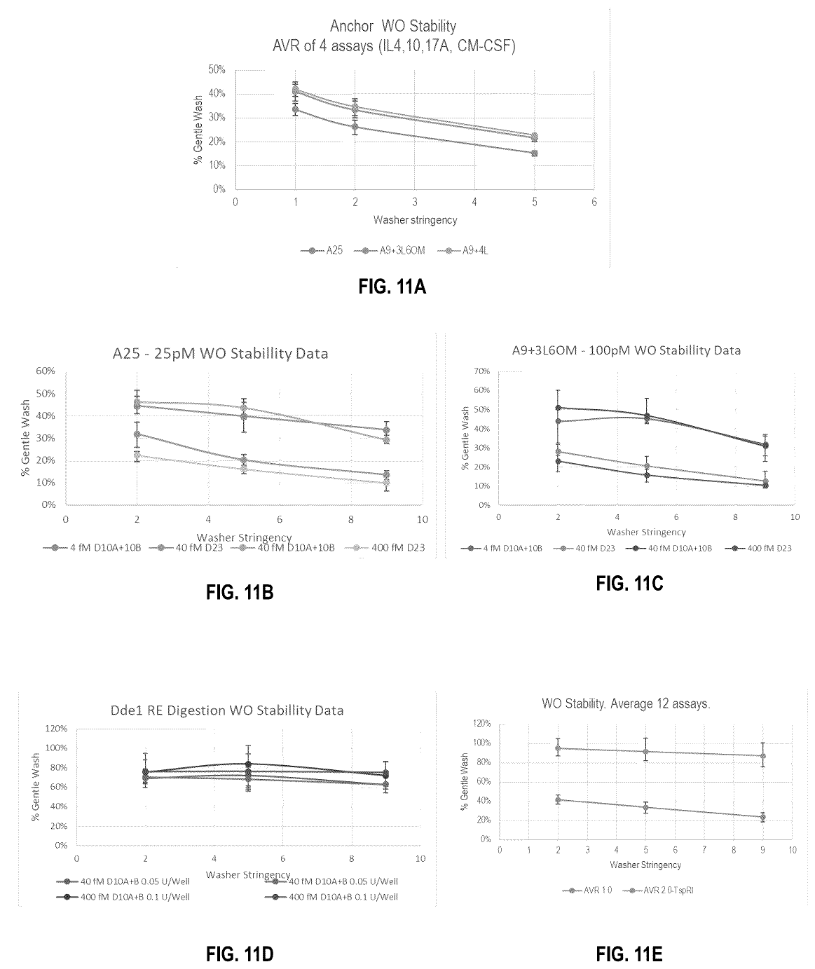

FIG. 11A shows representative results of the wash out stability in a standard sandwich immunoassay mediated by different anchoring oligonucleotides (“anchors”) comprising conventional 25-mer anchor (A25), modified 9-mer anchor with three LNAs and six 2′-OMe nucleotides (A9+3L6OM), and modified 9-mer anchor with four LNAs (A9+4L), each immobilized on the surface via binding of a biotin moiety. Four different immunoassays were tested with different anchors and conventional detection oligonucleotides, and results were averaged for all four assays. The modified anchoring oligonucleotides showed improved wash stability as compared to the convention anchoring oligonucleotide.

FIG. 11B shows representative results of an conventional anchoring 25-mer oligonucleotide without modifications with conventional detection oligonucleotide (“D23”) or with a mixture of modified detection oligonucleotides comprising 5 LNA and 5 2′-OMe modified nucleic acids (“D10A”) and modified detection oligonucleotides comprising 6 LNA and no 2′-OMe modified nucleic acids (“D10B”) as described herein (mixture denoted as “D10A+10B”), and varying concentrations of the primer oligonucleotide used in the simplified version of sandwich assay. The modified detection oligonucleotides showed improved wash stability as compared to the convention detection oligonucleotide.

FIG. 11C shows representative results of an anchoring oligonucleotide comprising nine nucleotides, three of which are LNAs and six of which are 2′-OMe nucleotides with D23 or D10A+D10B detection oligonucleotides. The modified detection oligonucleotides showed improved wash stability as compared to the convention detection oligonucleotide.

FIG. 11D shows representative results of an assay performed with template oligonucleotide cleavage with two concentrations of DdeI and two concentrations of the primer oligonucleotide used in the simplified version of sandwich assay as described in Example 2, and D10A+D10B detection oligonucleotides. All four conditions showed comparable wash stability which is significantly improved as compared to the condition without termination by template cleavage (FIG. 11C).

FIG. 11E shows representative results of the two-antibody sandwich immunoassay according to embodiments herein was performed on an MSD® streptavidin-coated plate when conventional anchor and detect oligonucleotides were used (AVR 1.0) in comparison to the modified anchor oligonucleotide (A9+3L6OM) immobilized to the surface via a thiol moiety, modified detection oligonucleotides (D10A+D10B) and template oligonucleotide cleavage with TspRI (AVR 2.0 TspRI). Signals generated from 12 assays were normalized and averaged for each combination of reagents, and results show significant improvement of wash stability with new modified oligonucleotides and termination by template cleavage as compared to the conventional oligonucleotides without termination.

### Example 6. Single Stranded Oligonucleotide (SSO) Stabilizing Agent During Extension

To test the effect of an SSO stabilizing agent during the extension reaction, Extreme Thermostable Single-Stranded DNA Binding Protein (ET SSB) was added in a benchmark assay based on the simplified assay as described in Example 2. A primer concentration corresponding to 500,000 molecules per well was used. The extension reaction was performed with Phi29 for 1 h, 4 h, or 24 h, in combined format with the “D10A+5L” detection oligonucleotide as described in Example 2. ET SSB was added to the plate before or after polymerase.

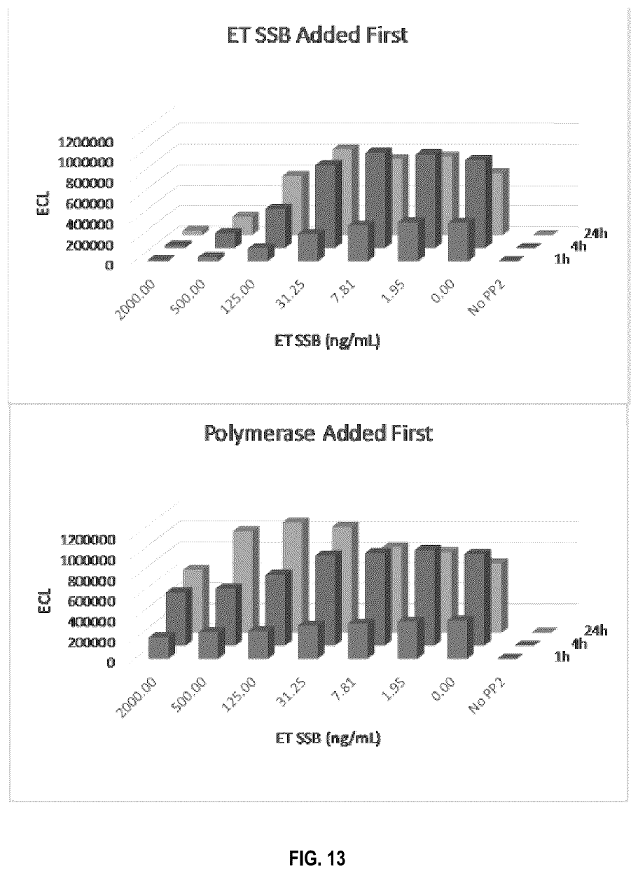

When SSB is added before the polymerase, signal recovery was observed from 4 h to 24 h at 31.25 ng/mL ET SSB, but the 24 h signal did not exceed the 4 h signal. See FIG. 13, top panel. The higher concentrations of ET SSB appear to inhibit polymerase activity. When ET SSB is added after the polymerase, the signal at 24 h exceeded the signal at 4 h at 125 ng/mL ET SSB. See FIG. 13, bottom panel.

A further benchmark assay with 100,000 primers per well was performed using different concentrations of ET SSB. The extension reaction was performed with Phi29 in combined format with “D10A+5L” for 1 h, 4 h, or 24 h. ET SSB was added to the assay 0, 5, 15, or 60 min after the polymerase.

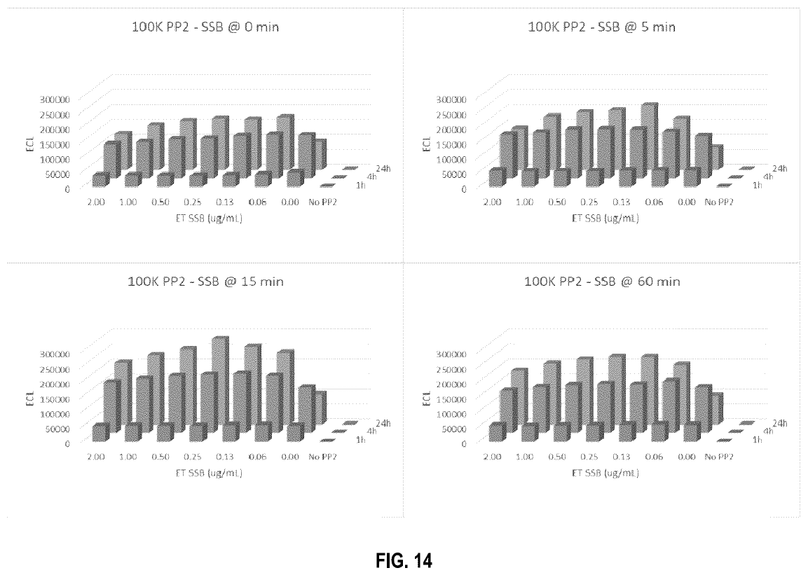

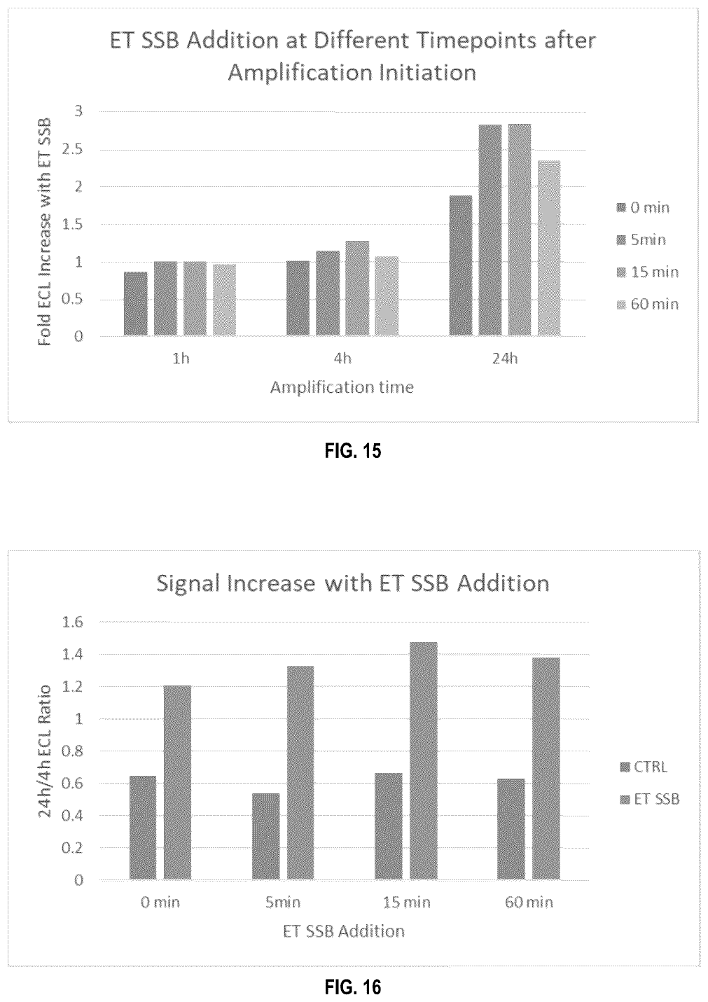

As shown in FIG. 14, at these assay conditions, ET SSB addition 15 min after initiation of the extension reaction displayed the strongest signal recovery. Further, ET SSB addition 15 min after initiation of the extension reaction increased the signal at 24 h 2.8-fold (FIG. 15) and by 47% compared to 4 h amplification (FIG. 16).

1. A method for detecting an analyte, comprising:
(a) contacting a template oligonucleotide with a first complex that comprises: (1) the analyte; and (2) a detection reagent that binds the analyte, wherein the detection reagent comprises a nucleic acid primer;
(b) hybridizing the nucleic acid primer to the template oligonucleotide to form a second complex and extending the nucleic acid primer with a polymerase, thereby forming an extended oligonucleotide;
(c) binding the extended oligonucleotide to one or more labeled probes, wherein each labeled probe comprises (1) a detection oligonucleotide that is capable of binding to the extended oligonucleotide; and (2) a detectable label; and
(d) detecting the detectable label, thereby detecting the analyte,
wherein the second complex is bound to a surface, and wherein:
(i) the detection oligonucleotide comprises RNA, a modified nucleic acid, or a combination thereof,
(ii) the method further comprises terminating the extending by cleaving the template oligonucleotide;
(iii) combination of (i) and (ii);
(iv) one or both of (i) and (ii), further wherein the surface comprises an anchoring reagent;
(v) the surface comprises an anchoring reagent that comprises an anchoring oligonucleotide, wherein the anchoring oligonucleotide comprises a modified nucleic acid; or
(vi) any combination of (i), (ii), and (v).

2. The method of claim 1, wherein the detection oligonucleotide comprises RNA, a modified nucleic acid, or a combination thereof, optionally wherein the modified nucleic acid comprises a peptide nucleic acid (PNA), a locked nucleic acid (LNA), a bridged nucleic acid (BNA), a nucleoside comprising a 2′ modification, or a combination thereof.

3. (canceled)

4. The method of claim 2, wherein the nucleoside comprising the 2′ modification comprises a 2′-O-methyl modification (2′-OMe), a 2′-O-methoxyethyl modification (2′-MOE), a 2′-deoxy-2′-fluoro modification (2′-F), a 2′-hydroxyl modification (2′-OH), or a combination thereof.

5. The method of claim 1, wherein the detection oligonucleotide is about 4 to about 30 nucleotides in length, or about 5 to about 25 nucleotides in length, or about 6 to about 12 nucleotides in length.

6-8. (canceled)

9. The method of claim 1, wherein the polymerase comprises strand-displacement activity (SD polymerase), optionally wherein the SD polymerase comprises an amino acid sequence of at least 80% sequence identity to a DNA polymerase from a bacteriophage, wherein the bacteriophage is Phi29, Nf, Karezi, or BeachBum.

10-12. (canceled)

13. The method of claim 1, wherein the template oligonucleotide is a linear oligonucleotide, and the method comprises ligating the 5′ and 3′ ends of the linear oligonucleotide prior to, during, or after hybridization of the nucleic acid primer to the template oligonucleotide, thereby forming a circular template.

14. The method of claim 1, wherein step (b) comprises: hybridizing the nucleic acid primer to the template oligonucleotide and extending the nucleic acid primer by nicking and extension amplification reaction (NEAR);
or wherein step (b) comprises: hybridizing the nucleic acid primer to the template oligonucleotide, ligating the template oligonucleotide to form a circular template, and extending the nucleic acid primer by rolling circle amplification (RCA).

15. (canceled)

16. The method of claim 1, wherein the method further comprises terminating the extending by cleaving the template oligonucleotide, optionally wherein the cleaving comprises contacting a nuclease with the template oligonucleotide, wherein the template oligonucleotide and/or the nuclease are characterized by any one of (I) to (XI):
(I) the nuclease specifically cleaves a double-stranded portion of the template oligonucleotide that is hybridized to the nucleic acid primer;
(II) the nuclease specifically cleaves a DNA/RNA hybrid that is formed from hybridization of the template oligonucleotide and the nucleic acid primer;
(III) the nuclease is a restriction endonuclease, optionally wherein the nuclease is DdeI, AluI, HpaII, ApoI, DpnI, DpnII, ScrFI, MboI, MluCI, AseI, TaqI, TspRI, or a combination thereof;
(IV) the nuclease is an endoribonuclease;
(V) the nuclease is RNase H2;
(VI) the template oligonucleotide comprises a DNA damage indicator, and the nuclease comprises an excision enzyme that specifically binds to the DNA damage indicator and cleaves the template oligonucleotide;
(VII) the template oligonucleotide comprises a DNA damage indicator, wherein the DNA damage indicator comprises an uracil base, and the nuclease comprises uracil-N-glycosylase (UNG);
(VIII) the template oligonucleotide comprises a DNA damage indicator, wherein the DNA damage indicator comprises an uracil base, the nuclease comprises uracil-N-glycosylase (UNG), and the cleaving further comprises providing an abasic site endonuclease, optionally wherein the abasic site endonuclease comprises Uracil-DNA glycosylase (UDG), APE1, Endonuclease IV, or a combination thereof;
(IX) the template oligonucleotide comprises a DNA damage indicator, wherein the DNA damage indicator comprises deoxyinosine, and the nuclease comprises Endonuclease V;
(X) the template oligonucleotide comprises a DNA damage indicator, wherein the DNA damage indicator comprises a damaged purine, and the nuclease comprises an enzyme that repairs the damaged purine;
(XI) the template oligonucleotide comprises a DNA damage indicator, wherein the DNA damage indicator comprises 8oxoG, and the nuclease comprises formamidopyrimidine DNA glycosylase (Fpg)

17-29. (canceled)

30. The method of claim 1, wherein the extending comprises rolling circle amplification (RCA), the method comprises the terminating, the method produces an assay signal range, and the assay signal range is characterized by any one of (I) to (III), and/or the extended oligonucleotide is characterized by any one of (IV) to (V):
(I) the assay signal range does not vary by more than 2-fold or more than 1.5-fold over an assay temperature range of about 20° C. to about 27° C.;
(II) the assay signal range does not vary by more than ±50% or more than ±30% over an assay temperature range of about 20° C. to about 27° C.;
(III) the assay signal range produced by the method does not vary by more than 2-fold over an extension time range of about 5 to about 90 minutes, or about 45 to about 90 minutes, or about 30 to about 60 minutes, or about 15 to 30 minutes, or about 10 to 20 minutes, or about 5 to 10 minutes;
(IV) the extended oligonucleotide is about 500 to about 50,000 bases in length, or about 9,000 to about 40,000 bases in length, or about 3,000 to about 13,000 bases in length, or about 1,000 to about 4,500 bases in length;
(V) the extended oligonucleotide comprises a length of about 5% to about 35%, or about 6% to about 32%, or about 1% to about 10% of an extended oligonucleotide formed by a substantially identical method that does not comprise the terminating.

31-49. (canceled)

50. The method of claim 1, wherein the surface comprises an anchoring reagent optionally comprising an anchoring oligonucleotide, wherein the anchoring oligonucleotide optionally comprises a modified nucleic acid, and further optionally wherein the modified nucleic acid comprises a PNA, an LNA, a BNA, a nucleoside comprising a 2′ modification, or a combination thereof.

51-53. (canceled)

54. The method of claim 50, wherein the nucleoside comprising the 2′ modification comprises a 2′-O-methyl modification (2′-OMe), a 2′-O-methoxyethyl modification (2′-MOE), a 2′-deoxy-2′-fluoro modification (2′-F), a 2′-hydroxyl modification (2′-OH), or a combination thereof.

55. The method of claim 50, wherein the extended oligonucleotide comprises an anchoring complement that is capable of binding to the anchoring oligonucleotide, and wherein the method further comprises binding the extended oligonucleotide to the anchoring reagent, optionally wherein the extended oligonucleotide is bound to the anchoring reagent prior to or during step (c) of the method.

56. (canceled)

57. The method of claim 50, wherein the anchoring oligonucleotide is about 4 to about 30 nucleotides in length, or about 6 to about 25 nucleotides in length, or about 8 to about 12 nucleotides in length.

58-60. (canceled)

61. The method of claim 50, wherein the surface comprises carbon composite, and the anchoring reagent is covalently immobilized on the surface, optionally wherein the anchoring reagent comprises a first binding partner, the surface comprises a second binding partner, and the anchoring reagent is immobilized on the surface via an interaction of the first and second binding partners, and wherein the first and second binding partners are characterized by any one of (I) to (VI):
(I) the first and second binding partners comprise a binding pair selected from cross-reactive groups, complementary oligonucleotides, a receptor-ligand pair, an antigen-antibody pair, a hapten-antibody pair, an epitope-antibody pair, a mimotope-antibody pair, an aptamer-target molecule pair, hybridization partners, or an intercalator-target molecule pair;
(II) the first binding partner comprises biotin, and the second binding partner comprises streptavidin, avidin, an anti-biotin antibody, or a combination thereof;
(III) the first binding partner is linked to a nucleotide of the anchoring oligonucleotide;
(IV) the first binding partner is positioned at a 5′-end or a 3-end of the anchoring reagent;
(V) the first binding partner is positioned at a 5′-end or a 3-end of the anchoring reagent, and the anchoring reagent further comprises a spacer positioned between the first binding partner and the anchoring oligonucleotide;
(VI) the first binding partner is positioned at a 3-end of the anchoring reagent, and the anchoring reagent comprises a PEG spacer comprising about 2 to about 10 ethylene glycol units.

62-70. (canceled)

71. The method of claim 1, wherein the first complex further comprises a capture reagent that binds the analyte and wherein, prior to or during step (a), the first complex is formed by:
contacting a sample comprising the analyte with: first, the capture reagent, and second, the detection reagent; or
contacting a sample comprising the analyte with: first, the detection reagent, and second, the capture reagent; or
contacting a sample comprising the analyte with the capture reagent and the detection reagent simultaneously or substantially simultaneously,
optionally wherein the capture reagent and the detection reagent each independently comprises an antibody or antigen-binding fragment thereof, oligonucleotide, antigen, ligand, receptor, hapten, epitope, mimotope, or aptamer,
further optionally wherein the capture reagent and the detection reagent each comprises an oligonucleotide.

72. (canceled)

73. (canceled)

74. The method of claim 1, wherein the first complex and/or the second complex is contacted with the polymerase and the labeled probe simultaneously or substantially simultaneously.

75. (canceled)

76. The method of claim 1, characterized by any of (I) to (V):
(I): (i) the detection oligonucleotide comprises RNA, a modified nucleic acid, or a combination thereof, and
(ii) the method comprises terminating the extending by cleaving the template oligonucleotide,
optionally wherein the surface comprises an anchoring reagent,
(II): (i) the detection oligonucleotide comprises RNA, a modified nucleic acid, or a combination thereof; and
(ii) the surface comprises an anchoring reagent:
(III): (i) the method comprises terminating the extending by cleaving the template oligonucleotide; and
(ii) the surface comprises an anchoring reagent:
(IV): the anchoring reagent comprises an anchoring oligonucleotide, wherein the anchoring oligonucleotide comprises a modified nucleic acid;
(V): (i) the detection oligonucleotide comprises RNA, a modified nucleic acid, or a combination thereof;
(ii) the method comprises terminating the extending by cleaving the template oligonucleotide; and
(iii) the surface comprises an anchoring reagent that comprises an anchoring oligonucleotide, wherein the anchoring oligonucleotide comprises a modified nucleic acid.

77-80. (canceled)

81. The method of claim 1, wherein:
the anchoring oligonucleotide comprises SEQ ID NO: 11;
the detection oligonucleotide comprises any one of SEQ ID NOs:7-10;
the nucleic acid primer comprises SEQ ID NO:1; and/or
the template oligonucleotide comprises SEQ ID NO:5.

82-87. (canceled)

88. The method of claim 1, wherein:
the detectable label is capable of being measured by light scattering, optical absorbance, fluorescence, chemiluminescence, electrochemiluminescence (ECL), bioluminescence, phosphorescence, radioactivity, magnetic field, or a combination thereof; and/or
wherein the surface comprises a particle, or wherein the surface comprises a well of a multi-well plate;
optionally wherein the surface comprises an electrode, and wherein the detecting comprises applying a voltage waveform to the electrode to generate an ECL signal; or wherein, when the surface comprises a particle, the detecting comprises collecting the particle on an electrode and applying a voltage waveform to the electrode to generate an ECL signal.

89-94. (canceled)

95. A kit for detecting an analyte comprising, in one or more vials, containers, or compartments:
a. a capture reagent that binds the analyte;
b. a detection reagent that binds the analyte, wherein the detection reagent comprises a nucleic acid primer or is capable of being linked to a nucleic acid primer;
c. a labeled probe that comprises (1) a detection oligonucleotide and (2) a detectable label; and
d. a template oligonucleotide that is capable of hybridizing to the nucleic acid primer and that comprises a same sequence as the detection oligonucleotide;
wherein the detection reagent comprises a protein or polypeptide, and wherein:
(i) the detection oligonucleotide comprises RNA, a modified nucleic acid, or a combination thereof,
(ii) the kit further comprises a nuclease that is capable of cleaving the template oligonucleotide;
(iii) combination of (i) and (ii);
(iv) one or both of (i) and (ii), and wherein the kit further comprises an anchoring reagent;
(v) the kit further comprises an anchoring reagent that comprises an anchoring oligonucleotide, wherein the anchoring oligonucleotide comprises a modified nucleic acid; or
(vi) any combination of (i), (ii), and (v).

96-169. (canceled)

170. A composition for labeling a surface, comprising (I) or (II):
(I): a labeled probe that comprises (1) a detectable label; and (2) detection oligonucleotide that is capable of binding to an extended oligonucleotide that is bound to the surface,
wherein the extended oligonucleotide is formed by extension of a nucleic acid primer by a polymerase based on a template oligonucleotide, and wherein:
(i) the detection oligonucleotide comprises RNA, a modified nucleic acid, or a combination thereof,
(ii) the composition further comprises a nuclease that is capable of cleaving the template oligonucleotide;
(iii) combination of (i) and (ii);
(iv) one or both of (i) and (ii), and further wherein the extended oligonucleotide is bound to the surface via an anchoring reagent;
(v) the surface comprises an anchoring reagent that comprises an anchoring oligonucleotide, wherein the anchoring oligonucleotide comprises a modified nucleic acid; or
(vi) any combination of (i), (ii), and (v); or
(II): (a) a nucleic acid primer that is immobilized directly or indirectly on a surface;
(b) a template oligonucleotide comprising (1) a first region that is complementary to the nucleic acid primer; and (2) a second region that comprises a same sequence as a detection oligonucleotide;
(c) a polymerase; and
(d) a labeled probe comprising (1) a detectable label; and (2) detection oligonucleotide that is capable of binding to an extended oligonucleotide that is bound to the surface,
wherein an extended oligonucleotide is formed by extension of the nucleic acid primer by the polymerase based on the template oligonucleotide, and wherein:
(i) the detection oligonucleotide comprises RNA, a modified nucleic acid, or a combination thereof;
(ii) the composition further comprises a nuclease that is capable of cleaving the template oligonucleotide;
(iii) combination of (i) and (ii);
(iv) one or both of (i) and (ii), and further wherein the extended oligonucleotide is bound to the surface via an anchoring reagent;
(v) the surface comprises an anchoring reagent that comprises an anchoring oligonucleotide, wherein the anchoring oligonucleotide comprises a modified nucleic acid; or
(vi) any combination of (i), (ii), and (v).

171-183. (canceled)

184. A composition comprising any one of (I) to (III):
(I): a capture reagent, an analyte, a detection reagent that comprises a nucleic acid primer, a template oligonucleotide, a polymerase, and a nuclease, wherein:
the capture reagent is immobilized on the surface;
the capture reagent and the detection reagent are bound to the analyte;
the nucleic acid primer is hybridized to the template oligonucleotide;
the polymerase is capable of extending the nucleic acid primer; and
the nuclease is capable of cleaving the template oligonucleotide; or
(II): a capture reagent, an analyte, a detection reagent that comprises an extended oligonucleotide, and an anchoring reagent that comprises an anchoring oligonucleotide, wherein:
the capture reagent and the anchoring reagent are immobilized on the surface;
the capture reagent and the detection reagent are bound to the analyte;
the anchoring oligonucleotide comprises a modified nucleic acid selected from a PNA, an LNA, a BNA, a nucleoside comprising a 2′ modification, or a combination thereof, optionally wherein the nucleoside comprising the 2′ modification comprises a 2′-O-methyl modification (2′-OMe), a 2′-O-methoxyethyl modification (2′-MOE), a 2′-deoxy-2′-fluoro modification (2′-F), a 2′-hydroxyl modification (2′-OH), or a combination thereof; and
the extended oligonucleotide comprises an anchoring complement that is bound to the anchoring oligonucleotide; or
(III): a capture reagent, an analyte, a detection reagent that comprises an extended oligonucleotide, and a labeled probe that comprises a detection oligonucleotide, wherein:
the capture reagent and the detection reagent are bound to the analyte;
the detection oligonucleotide comprises RNA, a modified nucleic acid, or a combination thereof, wherein the modified nucleic acid is selected from a PNA, an LNA, a BNA, a nucleoside comprising a 2′ modification, or a combination thereof, optionally wherein the nucleoside comprising the 2′ modification comprises a 2′-O-methyl modification (2′-OMe), a 2′-O-methoxyethyl modification (2′-MOE), a 2′-deoxy-2′-fluoro modification (2′-F), a 2′-hydroxyl modification (2′-OH), or a combination thereof; and
the extended oligonucleotide is bound to the detection oligonucleotide.

185-192. (canceled)

193. An oligonucleotide comprising or consisting of any one of SEQ ID NOs:7-11, 12-15, and 17.

194-198. (canceled)

199. A method for detecting an analyte, comprising any one of (I) to (VIII):
(I):
(a) contacting a template oligonucleotide with a first complex that comprises: (i) the analyte; and (ii) a detection reagent that binds the analyte, wherein the detection reagent comprises a nucleic acid primer;
(b) hybridizing the nucleic acid primer to the template oligonucleotide to form a second complex and extending the nucleic acid primer with a polymerase, thereby forming an extended oligonucleotide;
(c) contacting the extended oligonucleotide with a single stranded oligonucleotide (SSO) stabilizing agent; and
(d) detecting the extended oligonucleotide, thereby detecting the analyte;
(II):
(a) contacting a template oligonucleotide with a first complex comprising (i) the analyte; (ii) a capture reagent that binds the analyte, wherein the capture reagent is linked to an anchoring reagent that comprises an anchoring oligonucleotide to form a capture-anchoring reagent, wherein the capture-anchoring reagent is immobilized or capable of being immobilized to a surface; and (iii) a detection reagent for the analyte and that comprises a nucleic acid primer,
(b) hybridizing the nucleic acid primer to the template oligonucleotide to form a second complex and extending the nucleic acid primer with a polymerase, thereby forming an extended oligonucleotide, wherein the extended oligonucleotide comprises an anchoring complement that is capable of binding to the anchoring oligonucleotide;
(c) binding the extended oligonucleotide to the anchoring reagent; and
(d) detecting the extended oligonucleotide, thereby detecting the analyte;
(III):
(a) contacting a template oligonucleotide with a first complex that comprises:
(i) the analyte;
(ii) a first detection reagent that binds to the analyte and that comprises a first nucleic acid probe;
(iii) a second detection reagent that binds to the analyte and that comprises a second nucleic acid probe; and
(iv) a bridging oligonucleotide, wherein a first portion of the bridging oligonucleotide is capable of binding to the first nucleic acid probe and a second portion of the bridging oligonucleotide is capable of binding to the second nucleic acid probe, and wherein the bridging oligonucleotide further comprises a nucleic acid primer;
(b) hybridizing the nucleic acid primer to the template oligonucleotide to form a second complex and extending the nucleic acid primer with a polymerase, thereby forming an extended oligonucleotide; and
(c) detecting the extended oligonucleotide, thereby detecting the analyte;
(IV):
(a) contacting a template oligonucleotide with a first complex that comprises (i) the analyte; and (ii) a detection reagent that binds the analyte, wherein the detection reagent comprises a nucleic acid primer;
(b) hybridizing the nucleic acid primer to the template oligonucleotide to form a second complex and extending the nucleic acid primer with a polymerase, thereby forming an extended oligonucleotide, wherein the extended oligonucleotide comprises a binding sequence capable of binding to an anchoring reagent,
wherein the anchoring reagent is immobilized on a surface, or wherein the first complex further comprises a capture reagent that binds the analyte, wherein the capture reagent is immobilized or capable of being immobilized to the surface, and wherein the anchoring reagent is linked to the capture reagent, and
wherein the binding sequence is capable of forming an aptamer or a tertiary oligonucleotide structure, and the anchoring reagent comprises a protein, an antibody, a hapten, or an affinity tag capable of binding to the aptamer or tertiary oligonucleotide structure:
(c) binding the binding sequence to the anchoring reagent; and
(d) detecting the extended oligonucleotide, thereby detecting the analyte;
(a) contacting a template oligonucleotide with a first complex that comprises (i) the analyte; and (ii) a detection reagent that binds the analyte, wherein the detection reagent comprises a nucleic acid primer;
(b) hybridizing the nucleic acid primer to the template oligonucleotide to form a second complex and extending the nucleic acid primer with a polymerase, thereby forming an extended oligonucleotide, wherein the extended oligonucleotide comprises a binding sequence capable of binding to an anchoring reagent,
wherein the anchoring reagent is immobilized on a surface, or wherein the first complex further comprises a capture reagent that binds the analyte, wherein the capture reagent is immobilized or capable of being immobilized to the surface, and wherein the anchoring reagent is linked to the capture reagent, and
wherein the binding sequence comprises a binding moiety, and the anchoring reagent comprises a protein or antibody capable of binding the binding moiety;
(c) binding the binding sequence to the anchoring reagent; and
(d) detecting the extended oligonucleotide, thereby detecting the analyte;
(VI):
(a) contacting a template oligonucleotide with a first complex comprising
(i) the analyte;
(ii) a first detection reagent that binds to the analyte and that comprises a first nucleic acid primer; and
(iii) a second detection reagent that binds to the analyte and that comprises a second nucleic acid primer,
wherein the template oligonucleotide comprises a first region that is hybridizable with the first nucleic acid primer and a second region that is hybridizable with the second nucleic acid primer;
(b) hybridizing the first and second nucleic acid primers to the template oligonucleotide to form a second complex;
(c) extending the first nucleic acid primer to form a first extended oligonucleotide, and extending the second nucleic acid primer to form a second extended oligonucleotide; and
(d) detecting the first and second extended oligonucleotides, thereby detecting the analyte;
(VII):
(a) contacting a template oligonucleotide with a first complex comprising (i) the analyte; (ii) a first detection reagent that binds to the analyte and that comprises a first nucleic acid primer; and (iii) a second detection reagent that bind to the analyte and that comprise a second nuclei acid primer, wherein the analyte is present on a surface;
(b) hybridizing the first and second nucleic acid primers to the template oligonucleotide to form a second complex; and extending the first and/or the second nucleic acid primer with a polymerase, thereby forming an extended oligonucleotide; and
(c) detecting the extended oligonucleotide, thereby detecting the analyte;
(VIII):
(a) contacting a template oligonucleotide with a first complex that comprises: (i) the analyte; and (ii) a detection reagent that binds the analyte, wherein the detection reagent comprises a nucleic acid primer;
(b) hybridizing the nucleic acid primer to the template oligonucleotide to form a second complex and extending the nucleic acid primer with a polymerase, thereby forming an extended oligonucleotide, wherein the extended oligonucleotide is capable of enzymatic activity; and
(c) detecting the enzymatic activity, thereby detecting the analyte.

200-239. (canceled)
